SEQUENCE LISTING (55 sequences)

SEQ ID NO: 1 - DNA, 354 bp, Homo sapiens
gccgcgatgagcggggagccggggcagacgtccgtagcgccccctcccgaggaggtcgagccgggcagtggggtccgcatcgtggtggagtactgtgaaccctgcggcttcgaggcgacctacctggagctggccagtgctgtgaaggagcagtatccgggcatcgagatcgagtcgcgcctcgggggcacaggtgcctttgagatagagataaatggacagctggtgttctccaagctggagaatgggggctttccctatgagaaagatctcattgaggccatccgaagagccagtaatggagaaaccctagaaaagatcaccaacagccgtcctccctgcgtcatcctgtga

SEQ ID NO: 2 - protein, 115 aa, Homo sapiens
MSGEPGQTSVAPPPEEVEPGSGVRIVVEYCEPCGFEATYLELASAVKEQYPGIEIESRLGGTGAFEIEINGQLVFSKLENGGFPYEKDLIEAIRRASNGETLEKITNSRPPCVIL

SEQ ID NO: 3 - DNA, 30 bp, Artificial Sequence
  Primer used in production of B2-microglobulin
atcgatatgtctcgctccgtggccttagct

SEQ ID NO: 4 - DNA, 45 bp, Artificial Sequence
  Primer used in B2-microglobulin
cggggtacctgacccaccgcctcccatgtctcgatcccacttaac

SEQ ID NO: 5 - DNA, 42 bp, Artificial Sequence
  Primer used in production of the VH cloning cassette and the CH1 domain of IgG1
cggggtaccggaggcggtgggtcaggcgcgcatatggtcacc

SEQ ID NO: 6 - DNA, 33 bp, Artificial Sequence
  Primer used in production of the VH cassette and the CH1 domain IgG1
cggggatccctatttcttgtccaccttggtgtt

SEQ ID NO: 7 - DNA, 729 bp, Artificial Sequence
  Chimeric B2-microglobulin-F(ab) fragment
atcgatatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggtgggtcaggtaccggaggcggtgggtcaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaatagggatccccg

SEQ ID NO: 8 - protein, 237 aa, Artificial Sequence
  Chimeric B2-microglobulin-F(ab) fragment
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGTGGGGSGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 9 - DNA, 33 bp, Artificial Sequence
  Primer used in Production of VH Cassette/CH1/Hinge Domain of IgG1
cggggatccctatgggcacggtgggcatgtgtg

SEQ ID NO: 10 - DNA, 776 bp, Artificial Sequence
  Chimeric B2-microglobulin-F(ab′)2 fragment
atcgatatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggtgggtcaggtaccggaggcggtgggtcaggcgcgcatatgtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccatagggatccccg

SEQ ID NO: 11 - protein, 253 aa, Artificial Sequence
  Chimeric B2-microglobulin-F(ab′)2 fragment
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGTGGGGSGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCP

SEQ ID NO: 12 - DNA, 33 bp, Artificial Sequence
  Primer used in Production of IgG1
cggggatccctatttacccggagacagggagag

SEQ ID NO: 13 - DNA, 1428 bp, Artificial Sequence
  Chimeric B2-microglobulin-Full IgG1
atcgatatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggtgggtcaggtaccggaggcggtgggtcaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatagggatccccg

SEQ ID NO: 14 - protein, 470 aa, Artificial Sequence
  Chimeric B2-microglobulin-Full IgG1
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGTGGGGSGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - DNA, 38 bp, Artificial Sequence
  Primer used in Production of CH1/VH Cassette
aattgcggccgcaaaccatgggatggagctgtatcatc

SEQ ID NO: 16 - DNA, 45 bp, Artificial Sequence
  Primer used in Production of CH1/VH Cassette
cggggtacctgacccaccgcctcctttcttgtccaccttggtgtt

SEQ ID NO: 17 - DNA, 38 bp, Artificial Sequence
  Primer used in Production of B2-microglobulin
cgggtaccggaggcggtgggtcaatgtctcgctccgtg

SEQ ID NO: 18 - DNA, 29 bp, Artificial Sequence
  Primer used in Production of B2-microglobulin
cgggatccttacatgtctcgatcccactt

SEQ ID NO: 19 - DNA, 780 bp, Artificial Sequence
  Chimeric F(ab)-B2-microglobulin with signal sequence
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaaggaggcggtgggtcaggtaccggaggcggtgggtcaatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttggtgttcagcaaggactggtctttctatctcttgtactacactgaattcacccccactgaaaaagatgagtatgcctgccgtgtgaaccatgtgactttgtcacagcccaagatagttaagtgggatcgagacatgtaaggatcccg

SEQ ID NO: 20 - protein, 252 aa, Artificial Sequence
  Chimeric F(ab)-B2-microglobulin with signal sequence
MGWSCIILFLVATATGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKGGGGSGTGGGGSMSRSVALAVLALLSLSGLEAIQRTPKIQVYSRHPAENGKSNFLNCYVSGFHPSDIEVDLLKNGERIEKVEHSDLVFSKDWSFYLLYYTEFTPTEKDEYACRVNHVTLSQPKIVKWDRDM

SEQ ID NO: 21 - DNA, 45 bp, Artificial Sequence
  Primer used in Production of VH Cassette/CH1/hinge
cggggtacctgacccaccgcctcctgggcacggtgggcatgtgtg

SEQ ID NO: 22 - DNA, 828 bp, Artificial Sequence
  Chimeric F(ab′)2-B2-microglobulin
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccaggaggcggtgggtcaggtaccggaggcggtgggtcaatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttggtgttcagcaaggactggtctttctatctcttgtactacactgaattcacccccactgaaaaagatgagtatgcctgccgtgtgaaccatgtgactttgtcacagcccaagatagttaagtgggatcgagacatgtaaggatcccg

SEQ ID NO: 23 - protein, 268 aa, Artificial Sequence
  chimeric F(ab′)2-B2-microglobulin
MGWSCIILFLVATATGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSGTGGGGSMSRSVALAVLALLSLSGLEAIQRTPKIQVYSRHPAENGKSNFLNCYVSGFHPSDIEVDLLKNGERIEKVEHSDLVFSKDWSFYLLYYTEFTPTEKDEYACRVNHVTLSQPKIVKWDRDM

SEQ ID NO: 24 - DNA, 45 bp, Artificial Sequence
  Primer used in Production of VH cassette/CH1/hinge
cggggtacctgacccaccgcctcctttacccggagacagggagag

SEQ ID NO: 25 - DNA, 1479 bp, Artificial Sequence
  chimeric full IgG1-B2-microglobulin
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcggtgggtcaggtaccggaggcggtgggtcaatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttggtgttcagcaaggactggtctttctatctcttgtactacactgaattcacccccactgaaaaagatgagtatgcctgccgtgtgaaccatgtgactttgtcacagcccaagatagttaagtgggatcgagacatgtaaggatcccg

SEQ ID NO: 26 - protein, 485 aa, Artificial Sequence
  chimeric full IgG1-B2-microglobulin
MGWSCIILFLVATATGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGTGGGGSMSRSVALAVLALLSLSGLEAIQRTPKIQVYSRHPAENGKSNFLNCYVSGFHPSDIEVDLLKNGERIEKVEHSDLVFSKDWSFYLLYYTEFTPTEKDEYACRVNHVTLSQPKIVKWDRDM

SEQ ID NO: 27 - DNA, 44 bp, Artificial Sequence
  Primer used in Production of VL Cloning Cassette and the Kappa Light Chain Constant Region
cggggtaccggaggcggtgggtcagctacaggcgtgcacttgac

SEQ ID NO: 28 - DNA, 39 bp, Artificial Sequence
  Primer used in Production of B2-microglobulin
cggggtaccggaggcggtgggtcaatccagcgtactcca

SEQ ID NO: 29 - DNA, 720 bp, Artificial Sequence
  Chimeric F(ab)-mature-B2-microglobulin
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaaggaggcggtgggtcaggtaccggaggcggtgggtcaatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttggtgttcagcaaggactggtctttctatctcttgtactacactgaattcacccccactgaaaaagatgagtatgcctgccgtgtgaaccatgtgactttgtcacagcccaagatagttaagtgggatcgagacatgtaaggatcccg

SEQ ID NO: 30 - protein, 232 aa, Artificial Sequence
  Chimeric F(ab)-mature B2-microglobulin
MGWSCIILFLVATATGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKGGGGSGTGGGGSIQRTPKIQVYSRHPAENGKSNFLNCYVSGFHPSDIEVDLLKNGERIEKVEHSDLVFSKDWSFYLLYYTEFTPTEKDEYACRVNHVTLSQPKIVKWDRDM

SEQ ID NO: 31 - DNA, 768 bp, Artificial Sequence
  Chimeric F(ab′)2-mature B2-microglobulin
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccaggaggcggtgggtcaggtaccggaggcggtgggtcaatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttggtgttcagcaaggactggtctttctatctcttgtactacactgaattcacccccactgaaaaagatgagtatgcctgccgtgtgaaccatgtgactttgtcacagcccaagatagttaagtgggatcgagacatgtaaggatcccg

SEQ ID NO: 32 - protein, 248 aa, Artificial Sequence
  Chimeric F(ab′)2-mature B2-microglobulin
MGWSCIILFLVATATGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPGGGGSGTGGGGSIQRTPKIQVYSRHPAENGKSNFLNCYVSGFHPSDIEVDLLKNGERIEKVEHSDLVFSKDWSFYLLYYTEFTPTEKDEYACRVNHVTLSQPKIVKWDRDM

SEQ ID NO: 33 - DNA, 1419 bp, Artificial Sequence
  Chimeric Full IgG1-mature B2-microglobulin
gcggccgcaaaccatgggatggagctgtatcatcctcttcttggtagcaacagctacaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcggtgggtcaggtaccggaggcggtgggtcaatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttggtgttcagcaaggactggtctttctatctcttgtactacactgaattcacccccactgaaaaagatgagtatgcctgccgtgtgaaccatgtgactttgtcacagcccaagatagttaagtgggatcgagacatgtaaggatcccg

SEQ ID NO: 34 - protein, 87 aa, Artificial Sequence
  Primer used in Production of a B fragment comprising the CMV epitope, an amino acid linker, and nucleotides 1-15 of the B2-microglobulin gene
TGGAGTACGCTGGATGCCTCCTGACCCACCTGAGCCTCCTGATCCGCCTCCCCCACCTCCAACCGTAGCCACCATGGGCACCAGGTT

SEQ ID NO: 35 - DNA, 21 bp, Artificial Sequence
  Primer used in Production of a C fragment comprising the body (minus the signal sequence) of the human B2-microglobulin gene
atccagcgtactccaaagatt

SEQ ID NO: 36 - DNA, 45 bp, Artificial Sequence; identical to SEQ ID NO: 4
  Primer used in Production of a C fragment comprising the body (minus the signal sequence) of the human B2-microglobulin gene

SEQ ID NO: 37 - DNA, 42 bp, Artificial Sequence; identical to SEQ ID NO: 5
  Primer used in Production of the D fragment comprising the cloning site for VH and the coding sequence for CH1 of human IgG

SEQ ID NO: 38 - DNA, 33 bp, Artificial Sequence; identical to SEQ ID NO: 6
  Primer used in Production of the D fragment comprising the cloning site for VH and the coding sequence for CH1 of human IgG

SEQ ID NO: 39 - DNA, 803 bp, Artificial Sequence
  CMV-B2-microglobulin-CH1 Chimera
ccatcgatatgtctcgctccgtggccttagctgtgctcgcgctactctctctttctggcctggaggctaacctggtgcccatggtggctacggttggaggtgggggaggcggatcaggaggctcaggtgggtcaggaggcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggtgggtcaggtaccggaggcggtgggtcaggcgcgcatatggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaatagggatccccg

SEQ ID NO: 40 - protein, 261 aa, Artificial Sequence
  CMV-B2-microglobulin-CH1 Chimera
MSRSVALAVLALLSLSGLEANLVPMVATVGGGGGGSGGSGGSGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGTGGGGSGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKK

SEQ ID NO: 41 - DNA, 33 bp, Artificial Sequence; identical to SEQ ID NO: 9
  Primer used for F(ab′)2 fusion

SEQ ID NO: 42 - DNA, 33 bp, Artificial Sequence; identical to SEQ ID NO: 12
  Primer for IgG immunoglobulin heavy chain fusion

SEQ ID NO: 43 - protein, 465 aa, Artificial Sequence
  Chimeric full IgG1-mature B2-microglobulin
MGWSCIILFLVATATGAHMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGTGGGGSIQRTPKIQVYSRHPAENGKSNFLNCYVSGFHPSDIEVDLLKNGERIEKVEHSDLVFSKDWSFYLLYYTEFTPTEKDEYACRVNHVTLSQPKIVKWDRDM

SEQ ID NO: 44 - DNA, 30 bp, Artificial Sequence
  Primer used in production of a D fragment comprising the cloning site for VL and the kappa light chain constant region
cgggatccctaacactctcccctgttgaag

SEQ ID NO: 45 - DNA, 836 bp, Artificial Sequence
  CMV-B2-microglobulin-kappa light chain Chimera
ccatcgatatgtctcgctccgtggccttagctgtgctcgcgctactctctctttctggcctggaggctaacctggtgcccatggtggctacggttggaggtgggggaggcggatcaggaggctcaggtgggtcaggaggcatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggtgggtcaggtaccggaggcggtgggtcagctacaggcgtgcacttgactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagggatcccg

SEQ ID NO: 46 - protein, 271 aa, Artificial Sequence
  CMV-B2-microglobulin-kappa light chain Chimera
MSRSVALAVLALLSLSGLEANLVPMVATVGGGGGGSGGSGGSGGIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGTGGGGSATGVHLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 48 - DNA, 30 bp, Artificial Sequence; identical to SEQ ID NO: 44
  Primer used in Production of VL Cloning Cassette and the kappa light chain constant region

SEQ ID NO: 49 - DNA, 762 bp, Artificial Sequence
  Chimeric B2-microglobulin-kappa light chain
atcgatatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgggaggcggtgggtcaggtaccggaggcggtgggtcagctacaggcgtgcacttgactcgagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagggatcccg

SEQ ID NO: 50 - protein, 247 aa, Artificial Sequence
  Chimeric B2-microglobulin-kappa light chain
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGTGGGGSATGVHLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - DNA, 83 bp, Artificial Sequence
  Primer used in Production of B2-microglobulin signal sequence
ccatcgatatgtctcgctccgtggccttagctgtgctcgcgctactctctctttctggcctggaggctaacctggtgcccatg

SEQ ID NO: 52 - DNA, 83 bp, Artificial Sequence
  Primer used in Production of B2-microglobulin signal sequence
catgggcaccaggttagcctccaggccagaaagagagagtagcgcgagcacagctaaggccacggagcgagacatatcgatgg

SEQ ID NO: 53 - DNA, 87 bp, Artificial Sequence
  Primer used in Production of B fragment comprising CMV epitope, amino acid linker, and nucleotides 1-15 of the B2-microglobulin gene
aacctggtgcccatggtggctacggttggaggtgggggaggcggatcaggaggctcaggtgggtcaggaggcatccagcgtactcca

SEQ ID NO: 54 - DNA, 44 bp, Artificial Sequence; identical to SEQ ID NO: 27
  Primer used in Production of D fragment comprising the cloning site for VL and the kappa light chain constant region

SEQ ID NO: 55 - protein, 10 aa, Human Immunodeficiency Virus
RGPGRAFVTI

(19) United States
(12) Patent Application Publication
(10) Pub. No.: US 2005/0042218 A1
(43) Pub. Date: Feb. 24, 2005

(54) MHC class I - peptide-antibody conjugates with modified beta2-microglobulin

(75) Inventor: Maurice Zauderer, Pitsford, NY (US)

Correspondence Address: STERNE, KESSLER, GOLDSTEIN & FOX PLLC, 1100 NEW YORK AVENUE, N.W., WASHINGTON, DC, 20005, US

(73) Assignee: Vaccinex, Inc.

(21) Appl. No.: 10/887,230

(22) Filed: Jul. 9, 2004

Related U.S. Application Data
(60) Provisional application No. 60/485,716, filed on Jul. 10, 2003.
(60) Provisional application No. 60/513,043, filed on Oct. 22, 2003.

(51) Int. Cl. A61K 39/395; A61K 39/00

(52) U.S. Cl. 424/144.1; 424/185.1

(57) ABSTRACT

The present invention is directed to a novel targeted vaccine delivery system, comprising one or more peptide-MHC Class I complexes linked through the β₂-microglobulin molecule to an antibody which is specific for a cell surface marker. The complexes of the invention contain a β₂-microglobulin that has been modified to have greater affinity to the α chain of MHC Class I than native βP₂-microglobulin. Alternatively, the complexes of the invention contain β₂-microglobulin fused or linked to the antigenic peptide. The complexes of the invention are useful for treating and/or preventing cancer, infectious diseases, autoimmune diseases, and/or allergies.

### CROSS-REFERENCE TO RELATED APPLICATIONS

The present application claims priority benefit of U.S. Provisional Appl. Nos. 60/485,716 filed Jul. 10, 2003 and 60/513,043, filed Oct. 22, 2003, the disclosures of both of which are incorporated by reference herein.

### STATEMENT REGARDING FEDERALLY-SPONSORED RESEARCH AND DEVELOPMENT

Not applicable.

### BACKGROUND OF THE INVENTION

1. Field of the Invention

The present invention relates to immunology. More specifically, the present invention relates to vaccines and methods for modifying immune responses.

2. Background Art

T lymphocytes are both key effector cells and key regulatory cells of the immune system. The ability to stimulate or inhibit specific T cell responses is a major goal for the immunotherapy of cancer, infectious diseases, and autoimmune diseases. T cell specificity is mediated by a T cell receptor (TCR) on the surface of the T cells. Each TCR is specific for a complex of a unique peptide epitope of a protein antigen associated with a major histocompatibility complex (MHC) molecule on the surface of a cell. There are two classes of MHC proteins which bind to TCRs in conjunction with peptide antigens: MHC Class I proteins, which are found on the membranes of all nucleated cells; and MHC Class II proteins, which are found only on certain cells of the immune system. The two major classes of T cells, only on certain cells of the immune system. The two major classes of T cells, CD8+ and CD4+, are selected to be specific for peptide epitopes that associate, respectively, with MHC Class I and Class II molecules on the antigen presenting cell. Polymorphism within each class of MHC molecule determines which peptide fragments bind with functional affinity to the MHC molecules expressed by a particular individual.

Peptide-MHC complexes have a relatively fast dissociation rate from the TCR. Multimeric peptide-MHC complexes have, as expected, been shown to have slower dissociation rates and are far more suitable than soluble monomeric complex for binding to receptors on a specific T cell. A technology for engineering tetrameric peptide-MHC complexes based on addition of biotin to the COOH-terminus of the MHC Class I heavy chain and high affinity association with tetrameric avidin has been developed (Altman, J. D., et al., Science 274:94-96 (1996)). A similar strategy has been adapted for MHC Class II molecules (Schmitt, L. et al., Proc. Natl Acad. Sci., USA. 96:6581-6586 (1999); Zarutskie, J. A. et al., Biochemistry 38:5878-5887 (1999)). Such molecules are referred to as peptide-MHC tetramers and are widely employed for staining of specific T cells. A different form of dimeric peptide-MHC complex has been shown to activate specific T cells in vitro (Hamad, A. R. A. et al., J. Exp. Med. 188:1633-1640 (1998)).

Binding of peptide-MHC complexes to T cells is, in general, not sufficient to induce T cell proliferation and differentiation. Additional co-stimulatory signals delivered through interactions between other membrane molecules of the T cell and the antigen presenting cell are required for optimal T cell activation. Indeed, signaling through T cell antigen receptor alone in the absence of costimulation can result in tolerization rather than activation.

Dendritic cells are a uniquely potent lineage of professional antigen presenting cell that express high membrane levels of both MHC and co-stimulatory molecules. A number of vaccine strategies target antigen presentation by dendritic cells through ex vivo introduction of antigen into dendritic cells or provision of GM-CSF and/or other cytokines together with a source of antigen in vivo in order to promote recruitment and maturation of dendritic cells at the site of antigen deposit. Ex vivo strategies require complex manipulations of patient materials which are time consuming and expensive. In vivo manipulations are limited by the efficiency with which dendritic cells are recruited and with which they take up, process, and present antigenic peptide to specific T cells.

Both T cells and activated dendritic cells express membrane differentiation antigens that can be targeted by specific antibodies. Some of the corresponding membrane molecules may deliver either positive or negative activation signals to the T cell or dendritic cell precursor. These include the T cell markers CD28 and CTLA-4 (CD 152) which are, respectively, thought to mediate positive and negative co-stimulator interactions. In contrast, the dendritic cell differentiation markers CD83, CMRF-44 and CMRF-56 are not known to have a specific function in membrane signaling. CD83, in particular, has been tested in a variety of experiments and never found to have an effect beyond target cell recognition.

Methods are available to target a specific ligand or regulatory molecule to an antigen positive cell by genetically linking the specificity domain of an antibody specific for that antigen to a particular ligand or cytokine. Fusion proteins encoded in this fashion may retain both antigen specificity and ligand or cytokine function. Examples of such reagents have been described in which the ligand coding sequence is linked to either the carboxyl or amino terminus of an antibody chain which may itself be either whole or truncated (Morrison, S. L. et al., Clin. Chem. 34:1668-1675 (1988); Shin, S. U. and Morrison, S. L., Meth. in Enzymol. 178:459-476 (1989); Porto, J. D. et al., Proc. Nat'l. Acad Sci. USA 90:6671-6675 (1993); Shin, S.-U. et al., J. Immunol. 158:4797-4804 (1997)). A particularly flexible construct has been described, in which an avidin molecule is linked to the carboxyl-terminus of the heavy chain of an antibody that can target the transferrin receptor and can, in principle, deliver any biotinylated ligand to the target cell (Penichet, M. L. etal., J. Immunol. 163:4421-4426(1993)).

The key requirements for construction of a delivery system that can target specific cells and tissues to deliver a ligand or cytokine are to identify an appropriate target molecule, select an antibody with a specificity domain with high affinity for that target molecule, and to link an effective concentration of ligand or cytokine to that antibody specificity domain. For the specific purpose of vaccine delivery, the relevant ligand is a specific peptide-MHC Class I complex, preferably in dimeric or multimeric form. Two types of constructs would be especially useful: 1) a delivery vehicle that could target professional antigen presenting cells, such as dendritic cells, or other cells, such as tumor cells, epithelial cells or fibroblasts, and deliver an effective concentration of peptide-MHC Class I complex to modulate (i.e., stimulate or inhibit) a specific T cell response; and 2) a delivery vehicle that could target T cells through either positive or negative regulatory molecules, CD28 and CTLA-4, or lymphokine receptor, CD25, on the T cell and simultaneously deliver an effective concentration of peptide-MHC Class I complex to signal through the specific TCR.

In view of the diversity of antigens expressed in cancer and in infectious or autoimmune disease, and the natural polymorphism of human MHC, effective use of such fusion proteins for immunotherapy would be greatly facilitated by the ability to flexibly couple different peptide-MHC complexes to one or more idendritic cell or T cell targeting specificities.

### BRIEF SUMMARY OF THE INVENTION

The present invention provides compounds useful for modulating, i.e., either inhibiting or stimulating, an immune response. The compound of the invention comprises one or more peptide-MHC Class I complexes linked to an antibody or fragment thereof specific for a cell surface marker.

In one embodiment, the peptide-MHC Class I complexes comprise an MHC Class I α chain or fragment thereof, a β₂-microglobulin molecule or fragment thereof, and an antigenic peptide bound in the MHC groove, wherein the peptide-MHC Class I complex is linked to the antibody or fragment thereof through the β₂-microglobulin molecule or fragment thereof. The β₂-microglobulin molecule or fragment thereof may be linked to either the amino or carboxyl terminus of the antibody, through the heavy or light chain of the antibody. The antibody may be lined to either the amino or carboxyl terminus of the β₂-microglobulin.

In certain embodiments, the β₂-microglobulin molecule or fragment thereof is altered or modified in such a way as to have greater affinity for the α chain of MHC Class I than native β₂-microglobulin. In other embodiments, the β₂-microglobulin molecule or fragment thereof is fused or linked to the antigenic peptide.

In certain embodiments, the antibody is specific for a cell surface marker of a professional antigen presenting cell, more particularly a dendritic cell. In other embodiments, the antibody is specific for a cell surface marker of a tumor cell, an epithelial cell or a fibroblast. In other embodiments, the antibody is specific for a cell surface marker of a T cell.

In certain embodiments, the antigenic peptide is derived from a cancer cell. In other embodiments, the antigenic peptide is derived from an infectious agent or an infected cell. In still other embodiments, the antigenic peptide is derived from an allergen or the target tissue of an autoimmune disease. In other embodiments, the antigenic peptide is synthetic.

Also provided are method of modulating, i.e., either stimulating or inhibiting, and immune response, comprising administering to an animal an effective amount of a compound or composition of the invention.

### BRIEF DESCRIPTION OF THE DRAWINGS/FIGURES

FIG. 1 shows and amino acid sequence (SEQ ID NO:47) of native human β₂-microblogulin.

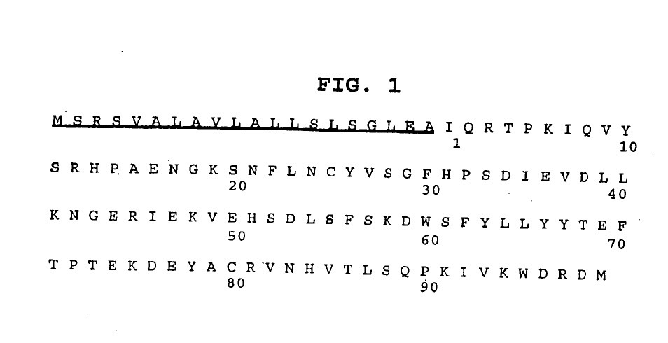

FIG. 2 shows the nucleotide (SEQ ID NO:1) and amino acid (SEQ ID NO:2) sequence of C35.

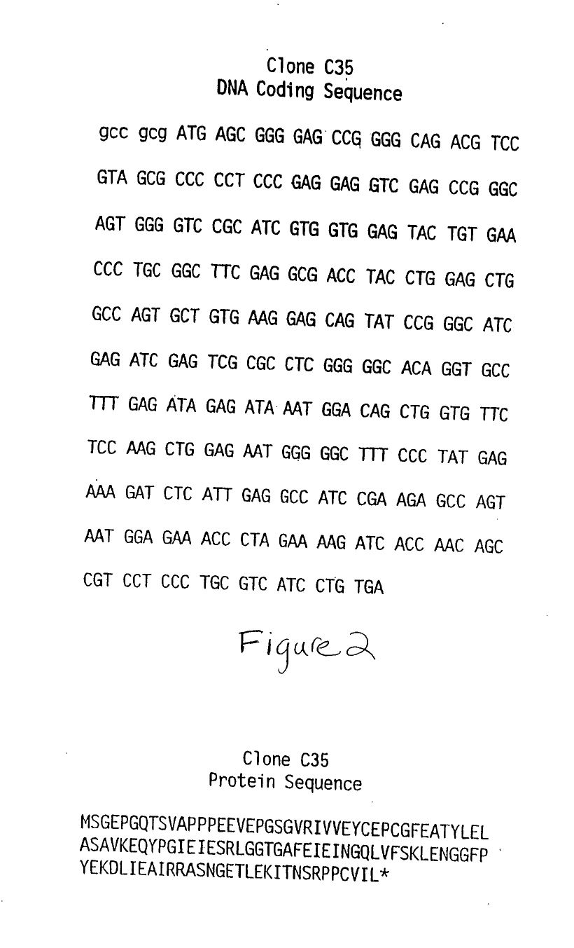

### DETAILED DESCRIPTION OF THE INVENTION

The present invention provides compounds which are useful for modulating, i.e., either inhibiting or stimulating, an immune response. The compounds comprise one or more peptide-MHC Class I complexes linked to an antibody or fragment thereof specific for a cell surface marker. The compounds are useful for stimulating desirable immune responses, for example, immune responses against infectious agents or cancer; or for inhibiting undesirable immune responses, such as allergic responses, allograft rejections, and autoimmune diseases. The present invention targets a peptide-MHC Class I complex to professional antigen presenting cells, such as dendritic cells, B cells, or macrophages; tumor cells; epithelial cells; fibroblasts; infected cells; T cells; or other cells, by linking one or more peptide-MHC complexes to an antibody or fragment thereof specific for a surface antigen of the targeted cell type. Depending on the targeted cell type, this will lead to either very efficient stimulation or inhibition of antigen specific T cell activity.

The term “MHC” encompasses similar molecules in different species. In mice, the MHC is termed H-2, in humans it is termed HLA for “Human Leucocyte Antigen.” When used herein, “MHC” is universally applied to all species.

MHC Class I molecules consist of an α (heavy) chain, coded for by MHC genes, associated with β₂-microglobulin, coded for by non-MHC genes. The β₂-microglobulin protein and α₃ segment of the heavy chain are associated; the α₁ and α₂ regions of the heavy chain form the base of the antigen-binding pocket (Science 238:613-614(1987); Bjorkman, P. J. et al., Nature 329:506-518 (1987)). An a chain may come from genes in the A, B or C subgroup. Class I molecules bind peptides of about 8-9 amino acids in length. All humans have between three and six different Class I molecules, which can each bind many different types of peptides.

Conventional identifications of particular MHC variants are used herein. For example, HLA-B17 refers to a human leucocyte antigen from the B gene group (hence a Class I type MHC) gene position (known as a gene locus) number 17.

MHC molecules useful in the present invention include, but are not limited to, HLA specificities such as A (e.g. A1-A74), B (e.g., B1-B77), and C (e.g., C1-C1). More preferably, HLA specificities include A1, A2, A3, A11, A23, A24, A28, A30, A33, B7, B8, B35, B44, B53, B60, and B62. It is possible to tissue type a person by serological or genetic analysis to define which MHC Class I variants each person has using methods known in the art.

The term “β₂-microblogulin” encompasses any β₂-microblogulin molecule, regardless of species. The sequence of polynucleotides encoding β₂-microblogulin, and the sequences of the β₂-microblogulin molecules themselves are known in the art. Examples include those sequences described in Parnes and Seidnam, Cell 29:661-669 (1982); Gates et al., PNAS USA 78:554-558 (1981); Suggs et al., PNAS USA 78:6613-6617 (1981); Guessow et al., J. Immunol. 139:3132-3128 (1987); Cunningham et al., Biochem. 12:4811-4822 (1983) and Ellis et al., Immunogenetics 38:310 (1993). Preferred are murine and human β₂-microblogulin. Particularly preferred is human β₂-microblogulin. “Native” or “wild-type” β₂-microblogulin refers to the β₂-microblogulin that is naturally occurring in an organism or typically found in nature.

The compounds of the invention comprise one or more peptide-MHC Class I complexes linked to an antibody or fragment thereof specific for a cell surface marker, wherein the peptide-MHC Class I complexes comprises an MHC Class I α chain or fragment thereof, a β₂-microglobulin molecule or fragment thereof, and an antigenic peptide bound in the MHC groove. In certain preferred embodiments, the peptide-MHC Class I complex is linked to the antibody through the β₂-microglobulin molecule or fragment thereof. This type of construct is particularly advantageous because it avoids the need to synthesize a different antibody-MHC fusion protein for each of many polymorphic MHC molecules. Since β₂-microglobulin is non-polymorphic, the same antibody-β₂-microglobulin fusion product can be made and employed to associate with multiple different MHC Class I alpha heavy chains.

The β₂-microglobulin molecule or fragment thereof may be linked to the heavy chain of the antibody or fragment thereof; or it may be linked to the light chain of the antibody or fragment thereof. In one embodiment, the compound of the invention contains β₂-microblogulin linked to both the heavy and light chains of the antibody or fragment thereof.

The β₂-microglobulin molecule or fragment thereof may be linked to the carboxyl terminus, or the amino terminus of the antibody; or it may be linked at a site other than the carboxyl or amino terminus of the antibody. Preferably, the β₂-microblogulin is linked to the carboxyl terminus of the heavy and/or light chain of the antibody. This embodiment minimizes the risk that the fusion peptide will interfere with the antibody binding site.

Examples 1-3 show a β₂-microglobulin molecule fused at the amino terminus of an antibody heavy or light chain or fragment thereof. Examples 4-6 show a β₂-microblogulin molecule fused at the carboxyl terminus of an antibody heavy or light chain or fragment thereof. Example 9 shows a β₂-microblogulin fused to the amino terminus of an antibody light chain. Example 11shows an antigenic peptide fused to the amino terminus of β₂-microblogulin, which is in turn fused to the amino terminus of a heavy chain of an antibody. Example 12 shows an antigenic peptide fused to the amino terminus of β₂-microblogulin, which is in turn fused to the amino terminus of a light chain of an antibody.

The antibody may be linked to the β₂-microglobulin molecule or fragment thereof at the carboxyl terminus, or the amino terminus of the β₂-microblogulin; or it may be linked at a site other than the carboxyl or amino terminus of the β₂-microglobulin.

The conjugation of the β₂-microglobulin to the antibody may be conducted in any suitable manner. For example, the coupling may be of a physical and/or chemical type. The antibody and β₂-microglobulin may be coupled physically utilizing a carrier for example a Sepharose carrier (available from Pharmacia, Uppsala, Sweden) or recently developed microsphere technology. (Southern Research Institute).

Alternatively, the β₂-microglobulin may be linked to the antibody directly. A number of reagents capable of cross-linking proteins are known in the art, illustrative entities include: azidobenzoyl hydrazide, N-[4-(p-azidosalicylamino)butyl]-3′-[2′-pyridyldithio]propionamide), bis-sulfosuccinimidyl suberate, dimethyladipimidate, disuccinimidyltartrate, N-γ-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, formaldehyde and succinimidyl 4-[N-maleimidomethyljcyclohexane-1-carboxylate.

Alternatively, the β₂-microglobulin can be genetically modified by including sequences encoding amino acid residues with chemically reactive side chains such as Cys or His. Such amino acids with chemically reactive side chains may be positioned in a variety of positions of β₂-microglobulin, preferably distal to the MHC Class I α binding domain. Suitable side chains can be used to chemically link two or more β₂-microglobulins to a suitable dendrimer particle. Dendrimers are synthetic chemical polymers that can have any one of a number of different functional groups on their surface (D. Tomalia, Aldrichimica Acta 26:91:101 (1993)). Exemplary dendrimers for use in accordance with the present invention include e.g. E9 starburst polyamine dendrimer and E9 combburst polyamine dendrimer, which can link cysteine residues.

Methods of making MHC-antibody fusion proteins are described in, for example, Dal Porto et al., Proc. Natl. Acad. Sci. USA 90:6671-6675 (1993) and Hamad et al., J. Exp Med. 188:1633-1640 (1998).

A short linker amino acid sequence may be inserted between the β₂-microglobulin and the antibody. The length of the linker sequence will vary depending upon the desired flexibility to regulate the degree of antigen binding and cross-linking. If a linker sequence is included, this sequence will preferably contain at least 3 and not more than 30 amino acids. More preferably, the linker is about 5, 10, 15, 20, or 25 amino acids long. Generally, the linker consists of short glycine/serine spacers, but any known amino acid may be used.

There may be one, two, three or four peptide-MHC Class I complexes per antibody. Preferably, there are two peptide-MHC Class I complexes per antibody. The attachment of the β₂-microglobulin to the antibody chains may be direct, i.e., without any intermediate sequence, or through a linker amino acid sequence, a linker molecule, or a chemical bond.

In certain embodiments, the β₂-microglobulin molecule or fragment thereof is altered or modified in such a way as to have greater affinity for the α chain of MHC Class I than native β₂-microglobulin. In one embodiment, the β₂-microblogulin molecule is a human β₂-microblogulin with a serine to valine mutation at position 55 of mature β₂-microblogulin, as described in, for example, WO 99/64597. In another embodiment, the β₂-microblogulin is human and the serine at position 55 of the mature form of human β₂-microblogulin has been replaced with a hydrophobic amino acid residue besides valine, e.g. isoleucine or leucine, particularly isoleucine; or a small side chain amino acid, e.g., alanine, threonine, methionine or glycine; or an aromatic amino acid, e.g. phenylalanine, tryptophan or tyrosine, especially phenylalanine; or a polar amino acid, e.g. glutamine or asparagine; or a basic amino acid, e.g. arginine, lysine or histidine; or an acid amino acid, e.g. aspartic acid or glutamic acid.

In other embodiments, the β₂-microblogulin or fragment thereof is fused or linked to the antigenic peptide. Methods of making fusion proteins comprising β₂-microblogulin and an antigenic peptide are described, for example, in U.S. Appl. Publ. No. 2002/0123108. This complex has greater affinity for the α chain of MHC Class I than native β₂-microblogulin without a peptide. The antigenic peptide can be linked to the amino or carboxyl terminus of the β₂-microblogulin molecule, or at a site other than the amino or carboxyl terminus. Preferably, the antigenic peptide is linked to amino terminus of the β₂-microblogulin. The β₂-microblogulin and antigenic peptide may be fused directly, i.e., without any intermediate linker; or there may be a linker peptide in between the β₂-microblogulin and antigenic peptide.

In certain embodiments, the β₂-microblogulin or fragment thereof is both modified to have more affinity for the α3 chain, for example, a S55V mutation, and is fused to an antigenic peptide.

In a preferred embodiment, the MHC Class I α subunit is a soluble form of the normally membrane-bound protein. The soluble form is derived from the native form by deletion of the transmembrane domain. The MHC molecules may also be truncated by removal of both the cytoplasmic and transmembrane domains. The protein may be truncated by proteolytic cleavage, or by expressing a genetically engineered truncated form.

For the a chain, the soluble form will include the α1, α2 and α3 domain. Not more than about 10, usually not more than about 5, preferably none of the amino acids of the transmembrane domain will be included. The deletion may extend as much as about 10 amino acids into the α3 domain, preferably none of the amino acids of the α3 domain will be deleted. The deletion will be such that it does not interfere with the ability of the α3 domain to fold into a disulfide bonded structure. β₂-microglobulin lacks a transmembrane domain in its native form, and need not be truncated. However, fragments of β₂-microglobulin are useful in the present invention.

One may wish to introduce a small number of amino acids at the polypeptide termini of the antibody, β₂-microglobulin or a chain of the MHC Class I, usually not more than 20, more usually not more than 15. The deletion or insertion of amino acids will usually be as a result of the needs of the construction, providing for convenient restriction sites, addition of processing signals, ease of manipulation, improvement in levels of expression, or the like. In addition, one may wish to substitute one or more amino acids with a different amino acid for similar reasons, usually not substituting more than about five amino acids in any one domain.

In embodiments where suppression or inhibition of an immune response is desired, for example, autoimmunity, allergies or transplant rejections, the MHC Class I α chain and/or β₂-microglobulin may be altered or mutated in such a way as to prevent associated with the CD8 costimulator molecule of T cells. It has been reported that binding to T cell receptor in the absence of CD8 interaction with other sites of MHC Class I molecule upregulates Fas ligand and promotes T cell apoptosis even in the absence of T cell activation. Mutations of the α chain are described, for example, in Salter et al., Nature 345:41-46 (1990). Mutations of the β₂-microglobulin molecule are described, for example, in Glick et al., J. Biol. Chem. 277:20840-20846 (2002) and WO 01/44296. If treatment of a particular patient with a positive stimulating compound of the invention resulted in some undesired inflammatory or autoimmune complication, then the administration of an inhibitory form of the same peptide-MHC Class I complex with a mutation that blocks association with CD8 can be used to reverse this effect.

In certain embodiments, the antigenic peptide is linked or fused to the MHC Class I α chain. Fusion of the antigenic peptide to the α chain increases the stability of the compounds of the invention. Methods of making antigenic peptide—α chain fisions are known in the art and disclosed, for example, in Mottez et al., J. Exp. Med. 181:493 (1995).

The α chain and β₂-microglobulin-fusion may be separately produced and allowed to associate to form a stable heteroduplex complex (see Altman et al. (1993), or Garboczi et al. (1992)), or both of the subunits may be expressed in a single cell. An alternative strategy is to engineer a single molecule having both the α chain and β₂-microglobulin-fusion. A “single-chain heterodimer” is created by fusing together the two subunits using a short peptide linker, e.g. a 15 to 25 amino acid peptide or linker. (Burrows G. G. et al., J. Immunology 161: 5987-5996 (1998)). See Bedzyk et al., J. Biol. Chem. 265:18615 (1990) for similar structures with antibody heterodimers. The soluble heterodimer may also be produced by isolation of a native heterodimer and cleavage with a protease, e.g. papain, to produce a soluble product.

The MHC molecules useful in the present invention may be from any mammalian or avian species, for example, primates (esp. humans), rodents, rabbits, equines, bovines, canines, felines, etc.

MHC molecules useful in the compounds of the present invention may be isolated from a multiplicity of cells, e.g., transformed cell lines JY, BM92, WIN, MOC, and MG, using a variety of techniques including solubilization by treatment with papain, by treatment with 3M KCl, and by treatment with detergent. Detergent can then be removed by dialysis or selection binding beads, e.g., Bio Beads.

Isolation of these detergent-soluble HLA antigens was described by Springer, T. A. et al., Proc Natl Acad Sci USA 73:2481-2485 (1976). Soluble HLA-A2 can be purified after papain digestion of plasma membranes from the homozygous human lymphoblastoid cell line J-Y as described by Turner, M. J. et al., J. Biol. Chem. 250:4512-4519 (1975); Parham P. et al., J. Biol. Chem. 252:7555-7567 (1977). Papain cleaves the 44 kd chain close to the transmembrane region yielding a molecule comprised of α₁, α₂, α₃ and β₂-microglobulin.

Alternatively, the amino acid sequence of a number of MHC proteins are known, and the genes have been cloned, therefore, the proteins can be made using recombinant methods. For example, the α chain of an MHC Class I molecule is synthesized using a truncation of the carboxyl terminus coding sequence which effects the deletion of the hydrophobic domain. The coding sequence for the α chain and β₂-microglobulin fusion are then inserted into expression vectors, expressed separately in an appropriate host, such as E. coli, yeast, insect cells, or other suitable cells. The recombinant α chain is recombined in the presence of the peptide antigen and the β₂-microglobulin-antibody fusion. Known, partial and putative HLA amino acid and nucleotide sequences, including the consensus sequence, are published (see, e.g., Zemmour and Parham, Immunogenetics 33:310-320 (1991)), and cell lines expressing HLA variants are known and generally available as well, many from the American Type Culture Collection (“ATCC”).

Antigenic peptides useful within the present invention include any peptide which is capable of modulating an immune response in an animal when presented in conjunction with an MHC molecule. Peptides may be derived from foreign antigens or from autoantigens.

The antigenic peptide will be from about 6 to 12 amino acids in length for complexes, usually from about 8 to 10 amino acids, most preferably 8 or 9 amino acids.

Methods for determining whether a particular peptide will bind to a particular MHC molecule are known in the art. See, for example, Parker et al., J. Immunol. 149:3580-3587 (1992); Southwood et al., J. Immunol. 160:3363-3373 (1998); Sturniolo et al., Nature Biotechnol. 17:5555-560 (1999).

The peptides may be loaded onto the MHC molecules in various forms. For example, a homogenous population of a known antigenic peptide may be added to the MHC in solution. Alternatively, a protein may be degraded chemically or enzymatically, for example, and added to the MHC molecules in this form. For example, a protein of interest is degraded with chymotrypsin and the resultant mixture of peptide “fragments” is added to the MHC molecules; the MHC are then allowed to “choose” the appropriate peptides to load onto the MHC molecules. Alternatively, mixtures of peptides from different proteins may be added to the MHC. For example, extracts from tumor cells or infected cells may be added to the MHC molecules in solution.

In certain embodiments, the antigenic peptide is fused or linked to the β₂-microglobulin molecule. In other embodiments, the antigenic peptide is fused or linked to the α chain of the MHC molecule.

Peptides according to the present invention may be obtained from naturally-occurring sources or may be synthesized using known methods. For example, peptides may be synthesized on an Applied Biosystems synthesizer, ABI 431A (Foster City, Calif.) and subsequently purified by HPLC. Alternatively, DNA sequences can be prepared which encode the particular peptide and may be cloned and expressed to provide the desired peptide. In this instance a methionine may be the first amino acid. In addition, peptides may be produced by recombinant methods as a fusion to proteins that are one of a specific binding pair, allowing purification of the fusion protein by means of affinity reagents, followed by proteolytic cleavage, usually at an engineered site to yield the desired peptide (see for example Driscoll et al., J. Mol. Bio. 232:342-350 (1993)). The peptides may also be isolated from natural sources and purified by known techniques, including, for example, chromatography on ion exchange materials, separation by size, immunoaffinity chromatography and electrophoresis.

Isolation or synthesis of “random” peptides may also be appropriate, particularly when one is attempting to ascertain a particular epitope in order to load an empty MHC molecule with a peptide most likely to stimulate T cells. One may produce a mixture of “random” peptides via use of proteasomes or by subjecting a protein or polypeptide to a degradative process—e.g., digestion with chymotrypsin—or peptides may be synthesized.

If one is synthesizing peptides, e.g., random 8- or 9-amino acid peptides, all varieties of amino acids are preferably incorporated during each cycle of the synthesis. It should be noted, however, that various parameters—e.g., solvent incompatibility of certain amino acids—may result in a mixture which contains peptides lacking certain amino acids. The process should thus be adjusted as needed—i.e., by altering solvents and reaction conditions—to produce the greatest variety of peptides.

In one embodiment, the antigenic peptide is derived from a cancerous cell, or promotes an immune response against a cancerous cell. In one embodiment, the antigenic peptide is derived from C35 (SEQ ID NOs:1 and 2).

A number of computer algorithms have been described for identification of peptides in a larger protein that may satisfy the requirements of peptide binding motifs for specific MHC Class I molecules. Because of the extensive polymorphism of MHC molecules, different peptides will often bind to different MHC molecules. Table 1 lists C35 peptides predicted for binding to the HLA Class I molecule HLA-A*0201 as well as a few limited examples of C35 peptides that express binding motifs specific for other selected Class I MHC molecules. Other C35 peptides which bind to specific HLA molecules are predicted in U.S. Appl. Publ. No. 2002/0155447, published Oct. 24, 2002, the disclosure of which is incorporated by reference herein.

TABLE 1
Predicted C35 HLA Class I epitopes*

| | |
|---|---|
| HLA restriction | Inclusive |

| | | |
|---|---|---|
| element | amino acids | Sequence |

| | | |
|---|---|---|
| A*0201 | 9-17 | SVAPPPEEV |
| A*0201 | 10-17 | VAPPPEEV |
| A*0201 | 16-23 | EVEPGSGV |
| A*0201 | 16-25 | EVEPGSGVRI |
| A*0201 | 36-43 | EATYLELA |
| A*0201 | 37-45 | ATYLELASA |
| A*0201 | 37-46 | ATYLELASAV |
| A*0201 | 39-46 | YLELASAV |
| A*0201 | 44-53 | SAVKEQYPGI |
| A*0201 | 45-53 | AVKEQYPGI |
| A*0201 | 52-59 | GIEIESRL |
| A*0201 | 54-62 | EIESRLGGT |
| A*0201 | 58-67 | RLGGTGAFEI |
| A*0201 | 61-69 | GTGAFEIEI |
| A*0201 | 66-73 | EIEINGQL |
| A*0201 | 66-74 | EIEINGQLV |
| A*0201 | 88-96 | DLIEAIRRA |
| A*0201 | 89-96 | LIEAIRRA |
| A*0201 | 92-101 | AIRRASNGET |
| A*0201 | 95-102 | RASNGETL |
| A*0201 | 104-113 | KITNSRPPCV |
| A*0201 | 105-113 | ITNSRPPCV |
| A*0201 | 105-114 | ITNSRPPCVI |
| A*3101 | 16-24 | EVEPGSGVR |
| B*3501 | 30-38 | EPCGFEATY |
| A*30101 supermotif | 96-104 | ASNGETLEK |

*predicted using rules found at the SYFPEITHI website (wysiwyg://35/http://134.2.96.221/scripts/hlaserver.dll/EpPredict.htm) and are based on the book “MHC Ligands and Peptide Motifs” by Rammensee, H.G., Bachmann, J. and S. Stevanovic. Chapman & Hall, New York, 1997.

Non-limiting examples of other peptides derived from cancer cells are described in Table 2.

TABLE 2
Peptides derived from cancer cells

| | Expressed | | | |
|---|---|---|---|---|
| Peptide Antigen(s) | in | MHC | HLA allele | Ref. |
| Melan A/MART-1 (26-35) | Melanoma | I | A*0201, | 1-3 |
| Gp 100 (71-78, 280-288) | Melanoma | I | A*0201, A11, | 5-7 |
| | | | A3, Cw8 | |
| Tyrosinase (368-376) | Melanoma | I | A*0201 | 8 |
| Tyrosinase related protein-2 | Melanoma | I | A*0201, A31, | 9-10 |
| (180-188, 197-205, | | | A33 (A3 st) | |
| 387-395) | | | | |
| MAGE-1 (multiple | Melanoma | I | A1, A2.1, | 11 |
| peptides) | | | A3.2, A11, | |
| | | | A24 | |
| MAGE-3 (168-176, | Melanoma | I | A*0101, | 12-13 |
| 271-279) | | | A*0201 | |

In another embodiment, the peptide is derived from an agent for infectious disease or an infected cell, or stimulates an immune response against an agent for infectious disease. Agents for infectious disease include bacteria, mycobacteria, fungi, worms, protozoa, parasites, viruses, prions, etc. Non-limiting examples of peptides derived from infectious agents are described in Table 3.

TABLE 3
Peptides derived from agents for infectious disease

| Peptide antigen | Expressed in | Rec. by | HLA allele | Ref. |
|---|---|---|---|---|
| CY1899 (core | Hepatitis B | I | A2*01 | 32-33 |
| protein 18-27) | | | | |
| NS4.1769 | Chronic hepatitis C | I | A2*01 | 36-37 |
| (NS4B, NS5B) | | | | |
| MN r gp 160 | HIV-1 | I | A2*01 | 39 |
| Tax (11-19) | HTLV-1 | I | A2*01 | 40 |
| MP (57-66) | Influenza | I | A2*01 | 41 |
| SSP2 | Malaria (Plasmodium | I | A2*01, | 43-44 |
| | falciparum) | | multiple A | |
| | | | and B | |
| | | | supertypes | |
| TSA-1, ASP-1, | Chagas' Disease | I | A2*01 | 45 |
| ASP-2 | (Trypanosoma cruzi) | | | |

Reference List for Tables 2 and 3:
1. Valmori, D. et al., J. Immunol. 161:6956-62 (1998).
2. Brinckerhoff, L. H. et al., Int. J. Cancer. 83:326-34 (1999).
3. Rivoltini, L. et al., Cancer Res. 59:301-6 (1999).
4. Zarour, H. M. et al., Proc. Natl. Acad. Sci USA. 97:400-5 (2000).
5. Castelli, C. et al., J. Immunol. 162:1739-48 (1999).
6. Abdel-Wahab, Z. et al., Cell. Immunol. 186:63-74 (1998).
7. Kawashima, I. et al., Int. J. Cancer. 78:518-24 (1998).
8. Valmori, D. et al., Cancer Res. 59:4050-5 (1999).
9. Parkhurst, M. R. et al., Cancer Res. 58:4895-901 (1998).
10. Wang, R. F. et al., J. Immunol. 160:890-7 (1998).
11. Celis, E. et al., Molecular Immunol. 31:1423-30 (1994).
12. Valmori, D. et al., Cancer Res. 57:735-41 (1997).
13. Fleischhauer, K. et al., J. Immunol. 159:2513-21 (1997).
32. Heatheote, J. et al., Hepatology 30:531-6 (1999).
33. Livingston, B. D. et al., J. Immunol 159:1383-92 (1997).
34. Bertoletti, A. et al., Hepatology 26:1027-34 (1997).
35. Diepolder, H. M. et al., J. Virol. 71:6011-9 (1997).
36. Alexander J. et al., Human Immunol. 59:776-82 (1998).
37. Battegay, M. et al., J. Virol. 69:2462-70 (1995).
39. Kundu, S. K. et al., AIDS Research and Human Retroviruses 14:1669-78 (1998).
40. Hollsberg, P. et al., Proc. Natl. Acad. Sci. USA. 92:4036-40 (1995).
41. Gotch, F. et al., Nature 326:881-2 (1987).
43. Doolan, D. L. et al., Immunity 7:97-112 (1997).
44. Wizel, B. et al., J. Immunol. 155:766-75 (1995).
45. Wizel, B. et al., J. Clin. Invest. 102:1062-71(1998).

The antigenic peptide may also be derived from a target tissue from autoimmune disease or from an allergen. Compounds comprising these antigenic peptides which suppress an immune response are especially preferred.

Further, the antigenic peptide may be synthetic. The synthetic peptide may provoke an immune response against cancerous cells or virus-infected cells. Alternatively, the synthetic peptide may downregulate an undesirable immune response, e.g, autoimmunity or allergy.

The sequence of antigenic peptide epitopes known to bind to specific MHC molecules can be modified at the known peptide anchor positions in predictable ways that act to increase MHC binding affinity. Such “epitope enhancement” has been employed to improve the immunogenicity of a number of different MHC Class I binding peptide epitopes (Berzofsky, J. A. et al., Immunol. Rev. 170:151-72 (1999); Ahlers, J. D. et al., Proc. Natl. Acad. Sci U.S.A. 94:10856-61 (1997); Overwijk, et al., J. Exp. Med. 188:277-86 (1998); Parkhurst, M. R. et al., J. Immunol. 157:2539-48 (1996)).

Antibodies are constructed of one, or several, units, each of which consists of two heavy (H) polypeptide chains and two light (L) polypeptide chains. The H and L chains are made up of a series of domains. The L chains, of which there are two major types (κ and λ), consists of two domains. The H chains molecules are of several types, including μ, δ, and γ (of which there are several subclasses), α and ε. In humans, there are eight genetically and structurally identified antibody classes and subclasses as defined by heavy chain isotypes: IgM, IgD, IgG3, IgG1, IgG2, IgG4, IgE, and IgA. Further, for example, “IgG” means an antibody of the G class, and that, “IgG1” refers to an IgG molecules of subclass 1 of the G class.

Thus, in certain embodiments, the antibody which is present in the compound of the invention is of the IgG1 isotype. In certain other embodiment,s the antibody is of the IgG2, IgG3, IgG4, IgA, IgM, IgD or IgE isotype. Particularly preferred is the IgG3, which has a longer and more flexible IgG3 hinge region.

As used herein, the term “antibody” (Ab) or “monoclonal antibody” (Mab) is meant to include intact molecules as well as antibody portions (such as, for example, Fab and F(ab′)₂ portions and Fv fragments) which are capable of specifically binding to a cell surface marker. Such portions are typically produced by proteolytic cleavage, using enzymes such as papain (to produce Fab portions) or pepsin (to produce F(ab′)₂ portions). Especially preferred in the compounds of the invention are Fab portions. Alternatively, antigen-binding portions can be produced through the application of recombinant DNA technology.

The immunoglobulin can be a “chimeric antibody” as that term is recognized in the art. Especially preferred for use in the present invention are chimeric monoclonal antibodies, preferably those chimeric antibodies having specificity toward a tumor associated antigen. As used in this example, the term “chimeric antibody” refers to a monoclonal antibody comprising a variable region, i.e. binding region, from one source or species and at least a portion of a constant region derived from a different source or species, usually prepared by recombinant DNA techniques. Chimeric antibodies comprising a murine variable region and a human constant region are preferred in certain applications of the invention, particularly human therapy, because such antibodies are readily prepared and may be less immunogenic than purely murine monoclonal antibodies. Such murine/human chimeric antibodies are the product of expressed immunoglobulin genes comprising DNA segments encoding murine immunoglobulin variable regions and DNA segments encoding human immunoglobulin constant regions. Other forms of chimeric antibodies encompassed by the invention are those in which the class or subclass has been modified or changed from that of the original antibody. Such “chimeric” antibodies are also referred to as “class-switched antibodies”. Methods for producing chimeric antibodies involve conventional recombinant DNA and gene transfection techniques now well known in the art. See, e.g., Morrison, S. L. et al., Proc. Nat'l Acad Sci. 81:6851 (1984).

Encompassed by the term “chimeric antibody” is the concept of “humanized antibody”, that is those antibodies in which the framework or “complementarity” determining regions (“CDR”) have been modified to comprise the CDR of an immunoglobulin of different specificity as compared to that of the parent immunoglobulin. In a preferred embodiment, a murine CDR is grafted into the framework region of a human antibody to prepare the “humanized antibody”. See, e.g., L. Riechmann et al., Nature 332:323 (1988); M. S. Neuberger et al., Nature 314:268 (1985). Particularly preferred CDR'S correspond to those representing sequences recognizing the antigens noted above for the chimeric and bifunctional antibodies. The reader is referred to the teaching of EPA 0 239 400 (published Sep. 30, 1987), for its teaching of CDR modified antibodies.

Also, the immunoglobulin may be a “bifunctional” or “hybrid” antibody, that is, an antibody which may have one arm having a specificity for one antigenic site, such as a tumor associated antigen while the other arm recognizes a different target, for example, a hapten which is, or to which is bound, an agent lethal to the antigen-bearing tumor cell. Alternatively, the bifinctional antibody may be one in which each arm has specificity for a different epitope of a tumor associated antigen of the cell to be therapeutically or biologically modified. In any case, the hybrid antibodies have a dual specificity, preferably with one or more binding sites specific for the hapten of choice or one or more binding sites specific for a target antigen, for example, an antigen associated with a tumor, an infectious organism, or other disease state.

Biological bifunctional antibodies are described, for example, in European Patent Publication, EPA 0 105 360, to which those skilled in the art are referred. Such hybrid or bifunctional antibodies may be derived, as noted, either biologically, by cell fusion techniques, or chemically, especially with cross-linking agents or disulfide bridge-forming reagents, and may be comprised of whose antibodies and/or fragments thereof Methods for obtaining such hybrid antibodies are disclosed, for example, in PCT application WO83/03679, published Oct. 27, 1983, and published European Application EPA 0 217 577, published Apr. 8, 1987. Particularly preferred bifunctional antibodies are those biologically prepared from a “polydome” or “quadroma” or which are synthetically prepared with cross-linking agents such as bis-(maleimideo)-methyl ether (“BMME”), or with other cross-linking agents familiar to those skilled in the art.

In addition the immunoglobin may be a single chain antibody (“SCA”). These may consist of single chain Fv fragments (“scFv”) in which the variable light (“V[L]”) and variable heavy (“V[H]”) domains are linked by a peptide bridge or by disulfide bonds. Also, the immunoglobulin may consist of single V[H ]domains (dAbs) which possess antigen-binding activity. See, e.g., G. Winter and C. Milstein, Nature 349:295 (1991); R. Glockshuber et al., Biochemistry 29:1362 (1990); and, E. S. Ward et al., Nature 341:544 (1989).

One skilled in the art will recognize that a bifunctional-chimeric antibody can be prepared which would have the benefits of lower immunogenicity of the chimeric or humanized antibody, as well as the flexibility, especially for therapeutic treatment, of the bifunctional antibodies described above. Such bifunctional-chimeric antibodies can be synthesized, for instance, by chemical synthesis using cross-linking agents and/or recombinant methods of the type described above. In any event, the present invention should not be construed as limited in scope by any particular method of production of an antibody whether bifunctional, chimeric, bifunctional-chimeric, humanized, or an antigen-recognizing fragment or derivative thereof.

In addition, the invention encompasses within its scope immunoglobulins (as defined above) or immunoglobulin fragments to which are fused active proteins, for example, an enzyme of the type disclosed in Neuberger et al., PCT application, WO86/01533, published Mar. 13, 1986. The disclosure of such products is incorporated herein by reference.

As noted, “bifunctional”, “fused”, “chimeric” (including humanized), and “bifunctional-chimeric” (including humanized) antibody constructions also include, within their individual contexts constructions comprising antigen recognizing fragments. As one skilled in the art will recognize, such fragments could be prepared by traditional enzymatic cleavage of intact bifunctional, chimeric, humanized, or chimeric-bifunctional antibodies. If, however, intact antibodies are not susceptible to such cleavage, because of the nature of the construction involved, the noted constructions can be prepared with immunoglobulin fragments used as the starting materials; or, if recombinant techniques are used, the DNA sequences, themselves, can be tailored to encode the desired “fragment” which, when expressed, can be combined in vivo or in vitro, by chemical or biological means, to prepare the final desired intact immunoglobulin “fragment”. It is in this context, therefore, that the term “fragment” is used.

Furthermore, as noted above, the immunoglobulin (antibody), or fragment thereof, used in the present invention may be polyclonal or monoclonal in nature. Monoclonal antibodies are the preferred immunoglobulins, however. The preparation of such polyclonal or monoclonal antibodies now is well known to those skilled in the art who, of course, are fully capable of producing useful immunoglobulins which can be used in the invention. See, e.g., G. Kohler and C. Milstein, Nature 256:495 (1975). In addition, hybridomas and/or monoclonal antibodies which are produced by such hybridomas and which are useful in the practice of the present invention are publicly available from sources such as the American Type Culture Collection (“ATCC”) 10801 University Boulevard, Manassas, Va. 20110-2209 or, commercially, for example, from Boehringer-Mannheim Biochemicals, P.O. Box 50816, Indianapolis, Ind. 46250.

The antibodies of the present invention may be prepared by any of a variety of methods. For example, cells expressing the cell surface marker or an antigenic portion thereof can be administered to an animal in order to induce the production of sera containing polyclonal antibodies. In a preferred method, a preparation of protein is prepared and purified as to render it substantially free of natural contaminants. Such a preparation is then introduced into an animal in order to produce polyclonal antisera of greater specific activity.

In the most preferred method, the antibodies of the present invention are monoclonal antibodies (or portions thereof). Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., In: Monoclonal Antibodies and T-Cell Hybridomas, Elsevier, N.Y., pp. 563-681 (1981)). In general, such procedures involve immunizing an animal (preferably a mouse) with a protein antigen or, more preferably, with a protein-expressing cell. Suitable cells can be recognized by their capacity to bind antibody. Such cells may be cultured in any suitable tissue culture medium; however, it is preferable to culture cells in Excell hybridoma medium (JRH Biosciences, Lenexa, Kans.) with 5% fetal bovine serum. The splenocytes of such immunized mice are extracted and fused with a suitable myeloma cell line. Any suitable myeloma cell line may be employed in accordance with the present invention; however, it is preferable to employ the parent myeloma cell line (SP₂O), available from the American Type Culture Collection, 10801 University Boulevard, Manassas, Va. 20110-2209. After fusion, the resulting hybridoma cells are selectively maintained in HAT medium, and then cloned by limiting dilution as described by Wands et al., Gastroenterology 80:225-232 (1981). The hybridoma cells obtained through such a selection are then assayed to identify clones which secrete antibodies capable of binding the antigen.

It may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

In one embodiment, the antibody is specific for a cell surface marker of a professional antigen presenting cell. Preferably, the antibody is specific for a cell surface marker of a dendritic cell, for example, CD83, CMRF-44 or CMRF-56. The antibody may be specific for a cell surface marker of another professional antigen presenting cell, such as a B cell or a macrophage. CD40 is expressed on both dendritic cells, B cells, and other antigen presenting cells so that a larger number of antigen presenting cells would be recruited.

In another embodiment, the antibody is specific for a cell surface marker of a T cell, for example, CD28, CTLA-4 (CD 152), or CD25. The combination of TCR mediated signal from the peptide-MHC complexes (signal 1) and co-stimulator signal through CD28 (signal 2) results in strong T cell stimulation. In contrast, the combination of TCR mediated signal from the peptide-MHC complexes (signal 1) and co-stimulator signal through CTLA-4 results in the inhibition of previously activated T cells or stimulation of antigen-specific inhibitors of activation of other T cells and may be especially useful for amelioration of autoimmune responses. CD25 is an IL-2 receptor upregulated upon T cell activation. Anti-CD25 fusion proteins could, therefore, specifically target T cells in an activated state.

CTLA-4 is a molecule expressed by activated T lymphocytes with very high affinity for costimulatory molecules B7-1 and B7-2 and has been reported to mediate signals that dampen or downregulate immune responsiveness (Bluestone, J. A. J. Immunol. 158:1989 (1997)). Although in most murine studies CTLA-4 specific antibodies have been reported to act antagonistically to block inhibitory effects, some human CTLA-4 specific monoclonal antibodies have been described that inhibit responses of resting human CD4+ T cells (Blair, P. J. et al., J. Immunol. 160:12-15 (1998)). The mechanisms of inhibition have not been fully characterized and may be mediated by either or both a direct inhibitory effect on T cells that have upregulated expression of CTLA-4 or through activation of a subset of inhibitory T cells that express high levels of CTLA-4. In either case, simultaneous binding of CTLA-4 and T cell receptor on a T cell by a CTLA-4 specific antibody linked to a polymeric complex of the cognate peptide-MHC Class I ligand may result in the inhibition of undesirable T cell reactivity for that peptide-MHC Class I complex. In one embodiment, a monovalent rather than polyvalent anti-CTLA-4 specificity may be linked to monomeric or polymeric peptide-MHC Class I complex.

T and B lymphocytes express a variety of surface molecules that, when crosslinked by antibodies, induce positive or negative signals that culminate in responsiveness or unresponsiveness. For the purpose of antigen delivery to T and B cells, it may, in some cases, be inadvisable to crosslink a cell surface antigen with divalent or polyvalent antibody since this may induce massive cell proliferation and splenomegaly in vivo (e.g. crosslinking CD3 or CD28 on T cells, or CD40 on B cells with specific antibody) or widespread cell death (anti-Fas antibody kills mice within hours of injection). Rather, it would be desirable simply to dock polymeric peptide-MHC Class I complexes on the lymphocyte surface using compounds of the invention with only monovalent antibody specificity. Additional strategies for linking multimeric peptide-MHC Class I complexes to either a monovalent or polyvalent antibody specificity are described below. The avidity of a specific T cell receptor for peptide-MHC Class I ligands of such complexes linked to an antibody with monovalent specificity for a T cell marker would be enhanced by polymeric binding of peptide-MHC Class I complexes as well as by linkage to the monovalent antibody specific for a second T cell membrane molecule. These targeted peptide-MHC Class I complexes can be employed to induce proliferation or cytotoxic activity of peptide-MHC Class I-specific T lymphocytes either in vitro or in vivo.

In another embodiment, the antibody is specific for a cell surface marker of a non-immune cell, for example, a tumor cell. Tumors evade the immune system in multiple ways, including downregulation of MHC Class I and Class II proteins on the surface. The compounds of the invention that specifically target tumor cells by virtue of antibody specific for antigens present on the tumor cell surface will increase presentation of peptide-MHC Class I ligands available for specific T cell recognition and activation. One tumor surface marker, C35, is described below.

Epithelial cells and fibroblasts are non-professional antigen presenting cells. Although they express MHC Class I molecules and can be induced to express MHC Class II after exposure to IFN-gamma, they are not fully competent to stimulate naïve T cells because they fail to express costimulatory molecules such as B7-1 and B7-2. Indeed, a signal throughthe T cell antigen receptor alone in the absence of a second costimulatory signal induces tolerance in naïve T cells. By targeting compounds of the invention to these non-professional antigen presenting cells, it should be possible to effectively induce tolerance to the immunodominant peptide-MHC Class I complexes of interest. A commercially available antibody, Ber-EP4 (Latza, U. et al., J. Clin. Pathol. 43:213-9 (1990), DAKO), reacts with two glycoproteins expressed on the surface of all epithelial cells except superficial squamous epithelial cells, hepatocytes, and parietal cells and has similar reactivity to HEA 125 (Moldenhauer, G. et al., Br. J. Cancer. 56:714-21 (1987)). Fibroblast-specific surface markers and antibodies that target them are under investigation in numerous laboratories and one potential candidate has been identified (Fearns, C and Dowdle, EB. Int. J. Cancer. 50:621-7 (1992), Miltenyi Biotech) that could be similarly employed to promote T cell unresponsiveness to linked monomeric or polymeric peptide-MHC Class I complexes. It is possible that for this specific application monomeric peptide-MHC Class I complexes that do not crosslink T cell receptors on the membrane of specific cells could prove more effective than polymeric peptide-MHC Class I complexes.

It has been reported that the liver is a site of accumulation of activated T lymphocytes about to undergo activation induced cell death (AICD) and that sinusoidal endothelial cells and Kupffer cells may constitute a “killing field” for activated CD8⁺ T cells originating from peripheral lymphoid organs (Mehal, Juedes and Crispe, J. Immunol. 163:3202-3210 (1999); Crispe, I. N. Immunol. Res. 19:143-57 (1999)). Compounds of the invention can promote trapping and deletion of specific T cells in the liver by targeting specific peptide-MHC Class I complexes to the liver with anti-hepatocyte specific antibodies.

In a preferred embodiment, the immune system's extraordinary power to eradicate pathogens is redirected to target an otherwise evasive tumor. The immune response to commonly encountered pathogens (eg influenza virus) and/or pathogens against which individuals are likely to have been vaccinated (eg influenza, or tetanus) is associated with induction of a high frequency of high avidity T cells that are specific for immunodominant peptide-MHC Class I complexes of cells infected with these pathogens. These same highly represented, high avidity T cells can be redirected to tumors by linking the dominant peptide-MHC Class I ligands recognized by these T cells to a tumor-specific antibody specificity. Redirection of specific T cell activity to tumor cells through antibody targeted peptide-MHC Class I complexes may proceed through two mechanisms. T cells either directly recognize antibody linked peptide-MHC Class I complexes displayed on the tumor surface, or such targeted complexes are internalized and the associated peptides are represented by MHC molecules endogenous to the tumor cell. Direct T cell recognition of the targeted complex can be demonstrated by employing T cells restricted to an MHC molecule that is not endogenous to the target cell.

Non-limiting examples of cell surface markers appropriate for immune targeting of the compounds of the present invention are presented in Tables 4 and 5.

TABLE 4
Human leukocyte differentiation antigens

| Surface Antigen | Expressed by | Ref. |
|---|---|---|
| CD2 | T lymphocytes | 1-2 |
| CD4 | T cell subset | 1 |
| CD5 | T lymphocytes | 1 |
| CD6 | T lymphocytes | 1, 3 |
| CD8 | T cell subset | 1 |
| CD27 | Naïve CD4 T cell subset | 4 |
| CD31 | Naïve CD4 T cell subset | 4 |
| CD25 | Activated T cells | 1 |
| CD69 | Activated T cells | 1, 5, 6 |
| HLA-DR | Activated T cells, APC | 7 |
| CD28 | T lymphocytes | 8 |
| CD152 (CTLA-4) | Activated T cells | 9 |
| CD154 (CD40L) | Activated T cells | 10 |
| CD19 | B lymphocytes | 1, 11 |
| CD20 | B lymphocytes | 1 |
| CD21 | B lymphocytes | 1 |
| CD40 | Antigen presenting cells | 12-13 |
| CD134 (OX40) | Antigen presenting cells | 13-14 |
| B7-1 and 2 | Antigen presenting cells | 13, 15, 16 |
| CD45 | Leukocytes | 1 |
| CD83 | Mature dendritic cells | 17 |
| CMRF-44 | Mature dendritic cells | 18 |
| CMRF-56 | Mature dendritic cells | 19 |
| OX40L | Dendritic cells | 20 |
| DEC-205 | Dendritic cells | 21 |
| TRANCE/RANK receptor | Dendritic cells | 22 |

Reference listing for table 4:
1. Knapp, W. et al., eds., Leukocyte Typing IV: White Cell Differentiation Antigens, Oxford University Press, New York. (1989).
2. Bierer, B. E. et al., Seminars in Immunology. 5:249-61(1993).
3. Rasmussen, R. A. et al., J. Immunol. 152:527 (1994).
4. Morimoto, C. et al., Clin. Exp. Immunol. 11:241-7 (1993).
5. Ziegler, S. F. et al., Stem Cells 12:456-65 (1994).
6. Marzio, R. et al., CD69 and regulation of immune function. 21:565-82 (1999).
7. Rea, I. M. et al., Exp. Gerontol. 34:79-93 (1999).
8. June, C. H. et al., Immunology Today 11:211 (1993).
9. Lindsten, T. et al., J. Immunol. 151:3489 (1993).
10. Mackey, M. F. et al., J. Leukocyte Biol. 63:418-28 (1998).
11. Bradbury, L. E. et al., J. Immunol. 151:2915 (1993).
12. Clark, E. A., and Ledbetter, J. A., Proc. Natl. Adad. Sci. USA. 83:4494 (1986).
13. Schlossman, S. et al., eds. Leukocyte Typing V: White Cell Differentiation Antigens. Oxford University Press, New York (1995).
14. Latza, U. et al., Eur. J. Immunol. 24:677 (1994).
15. Koulova, L. et al., J. Exp. Med. 173:759 (1991).
16. Azuma, M. et al., Nature 366:76 (1993).
17. Zhou, L. J., and Tedder, T. F., J. Immunol. 154: 3821 (1995).
18. Vuckovic, S. et al., Exp. Hematology 26:1255 (1998).
19. Hock, B. D. et al., Tissue Antigens 53:320-34 (1999).
20. Chen, A. I. et al., Immunity 11:689 (1999).
21. Kato, M. et al., Immunogenetics. 47:442 (1998).

22. Anderson, D. M. et al., Nature 390:175 (1997).

TABLE 5
Tumor cell surface antigens recognized by antibodies

| Antigen(s) | Expressed in | Ref. |
|---|---|---|
| CEA | Colorectal, thyroid carcinoma, others | 1-6 |
| Her2/neu | Breast, ovarian carcinomas | 7 |
| CM-1 | Breast | 8 |
| MUC-1 | Pancreatic carcinoma, others | 9-10 |
| 28K29 | Lung adenocarcinoma, large cell | 11 |
| | carcinoma | |
| E48 | Head and neck squamous cell carcinoma | 12 |
| U36 | Head and neck squamous cell carcinoma | 12 |
| NY-ESO-1* | Esophageal carcinoma, melanoma, | 13-14 |
| | others | |
| KU-BL 1-5* | Bladder carcinoma | 15 |
| NY CO 1-48* | Colon carcinoma | 16 |
| HOM MEL 40* | Melanoma | 17 |
| OV569 | Ovarian carcinoma | 18 |
| ChCE7 | Neuroblastoma, renal cell carcinoma | 19 |
| CA19-9 | Colon carcinoma | 20 |
| CA125 | Ovarian carcinoma | 21 |
| Gangliosides (GM2, | Melanoma, neuroblastoma, others | 22 |
| GD2, 9-o-acetyl- | | |
| GD3, GD3) | | |

*Antigens identified using SEREX technology.

Reference List for Table 5:
1. Juweid, M. E. et al., Cancer 85:1828-42 (1999).
2. Stewart, L. M. et al., Imunotherapy 47:299-306 (1999).
3. Robert, B. et al., International J. Cancer 81:285-91 (1999).
4. Kraeber-Bodere, F. et al., J. Nuclear Medicine 40:198-204 (1999).
5. Kawashima, I. et al., Cancer Res. 59:431-5 (1999).
6. Nasu, T. et al., Immunology Letters 67:57-62 (1999).
7. Zhang, H. et al., Experimental & Molecular Pathology 67:15-25 (1999).
8. Chen, L. et al., Acta Academiae Medicinae Sinicae 19(2):150-3.
9. Beum, P. V. et al., J. Biol. Chem. 274:24621-8 (1999).
10. Koumarianou A. A. et al., British J. Cancer 81:431-9 (1999).
11. Yoshinari, K. et al., Lung Cancer 25:95-103 (1999).
12. Van Dongen, G. A. M. S. et al., Anticancer Res. 16:2409-14 (1996).
13. Jager, E. et al., J. Exp. Med. 187:265-70 (1998).
14. Jager, E. et al., International J. Cancer 84:506-10 (1999).
15. Ito, K. et al., AUA 2000 Annual Meeting, Abstract 3291 (2000).
16. Scanlan, M. J. et al., International J. Cancer 76:652-8 (1998).
17. Tureci, O. et al., Cancer Res. 56:4766-72 (1996).
18. Scholler, N. et al., Proc. Natl. Acad, Sci. USA 96:11531-6 (1999).
19. Meli, M. L. et al., International J. Cancer 83:401-8 (1999).
20. Han, J. S. et al., Cancer 76:195-200 (1995).
21. O'Brien, T. J. et al., International J. Biological Markers 13:188-95 (1998).
22. Zhang, S. et al., Cancer Immunol. Immunotherapy 40:88-94(1995).

Described below are direct fusion of β₂-microglobulin molecules to the amino or carboxyl end of an antibody immunoglobulin chain or fragment thereof. Fusion of MHC molecules to the amino terminus of the immunoglobulin chain variable regions has been previously described (Dal Porto, J. et al., Proc. Natl. Acad. Sci., USA 90:6671-75 (1993)). Although this fusion product does not interfere with recognition of haptens in fusion products with hapten-specific antibody, the proximity of peptide-MHC Class I complex and antibody binding site makes it more likely that the peptide-MHC Class I complex could interfere with antibody binding to macromolecular determinants embedded in a complex membrane. Moreover, while fusion of MHC molecules to the amino terminus of immunoglobulin or immunoglobulin fragments preserves the Fc binding function for optimal presentation of peptide-MHC Class I complex by Fc receptor expressing cells, the relative orientation of antibody binding site and peptide-MHC Class I complex is far less favorable for antigen presentation to T cells by cells that might be targeted by the specific antibody (Hamad, A. R. A. et al., J. Exp. Med. 188:1633-40 (1998); Greten, T. F. et al., Proc. Natl. Acad. Sci., USA 95:7568-73 (1998); Casares, S. et al., J. Exp. Med. 190:543-553 (1999)). There is, therefore, a need for new compounds that can serve the requirements of targeted delivery of polymeric peptide-MHC Class I ligand to T cells and their antigen-specific receptor. Localization of the MHC molecule at the carboxyl terminus of immunoglobulin chains serves this purpose. The peptide-MHC Class I complex is well separated from the antibody binding site and is unlikely to interfere with its targeting specificity.

MHC molecules fused to the carboxyl terminus of the exceptionally long IgG3 hinge region or to the CH3 domain, are especially far removed from possible interference with the antigen binding site or its ligand. Moreover, the preferred embodiments of the compounds of this invention promote antibody mediated targeting to antigen presenting cells or tumors in a way which properly orients polymeric peptide-MHC Class I complexes for presentation to T cells and their antigen-specific receptors. Fc binding function is preserved in the compounds of this invention that are based on CH3 fusions. It is possible that this would extend the half-life of these compounds in vivo.

In one embodiment, the compound of the invention incorporates an antibody specificity for a particular immunoglobulin class or isotype, in a preferred embodiment this is an IgG isotype whose expression is regulated by cytokines secreted by Th1 type T cells, compounds of the invention with this immunoglobulin isotype specificity will bind antigen-specific humoral antibodies of this isotype. The bound humoral antibody will, as a result, target the linked peptide-MHC Class I complex and any linked cytokines to those cells that express the specific foreign antigens or autoantigens that were responsible for inducing this specific antibody response. The rationale is that, without prior knowledge of the specific antigens targeted in this cancer or infectious disease, it will be possible to deliver desired markers or signals to eradicate the cellular source of specific antigen.

The compound of the invention may further comprise a cytokine or lymphokine. The cytokine or lymphokine may be linked to the antibody or the peptide-MHC Class I complex. The cytokine or lymphokine may be linked to the antibody or the peptide-MHC Class I complex through an intermediate. Alternatively, the cytokine or lymphokine may be directly fused to the antibody or peptide-MHC Class I complex.

The term “cytokine” refers to polypeptides, including, but not limited to, interleukins (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, and IL-18), α interferons (e.g., IFNα), ω interferon (IFNω), β interferons (e.g., IFNβ), γ interferons (e.g., IFNγ), τ interferon (IFNτ), colony stimulating factors (CSFs, e.g., CSF-1, CSF-2, and CSF-3), granulocyte-macrophage colony stimulating factor (GMCSF), transforming growth factor (TGF, e.g.,.TGFα and TGFβ), and insulin-like growth factors (IGFs, e.g., IGF-I and IGF-II).

The compound of the invention may further comprise other therapeutic agents. The therapeutic agent or agents may be linked to the antibody or the peptide-MHC Class I complex. Examples of therapeutic agents include, but are not limited to, antimetabolites, alkylating agents, anthracyclines, antibiotics, and anti-mitotic agents. Antimetabolites include methotrexate, 6-mercaptopurine, 6-thioguanine, cytarabine, 5-fluorouracil decarbazine. Alkylating agents include mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP) cisplatin. Anthracyclines include daunorubicin (formerly daunomycin) and doxorubicin (also referred to herein as adriamycin). Additional examples include mitozantrone and bisantrene. Antibiotics include dactinomycin (formerly actinomycin), bleomycin, mithramycin, and anthramycin (AMC). Antimytotic agents include vincristine and vinblastine (which are commonly referred to as vinca alkaloids). Other cytotoxic agents include procarbazine, hydroxyurea, asparaginase, corticosteroids, mytotane (O,P′-(DDD)), interferons. Further examples of cytotoxic agents include, but are not limited to, ricin, doxorubicin, taxol, cytochalasin B, gramicidin D, ethidium bromide, etoposide, tenoposide, colchicin, dihydroxy anthracin dione, 1-dehydrotestosterone, and glucocorticoid.

Clearly analogs and homologs of such therapeutic and cytotoxic agents are encompassed by the present invention. For example, the chemotherapuetic agent aminopterin has a correlative improved analog namely methotrexate. Further, the improved analog of doxorubicin is an Fe-chelate. Also, the improved analog for 1-methylnitrosourea is lomustine. Further, the improved analog of vinblastine is vincristine. Also, the improved analog of mechlorethamine is cyclophosphamide.

The compound of the present invention may be labeled so as to be directly detectable, or will be used in conjunction with secondary labeled immunoreagents which will specifically bind the compound, for example, for detection or diagnostic purposes. The compound can be labeled through the MHC Class I α chain, the β₂-microglobulin molecule, the antigenic peptide or the antibody. Preferably, the antibody is labeled.

Suitable labels for the compound of the present invention are provided below. Examples of suitable enzyme labels include malate dehydrogenase, staphylococcal nuclease, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, ³⁵S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is a preferred isotope where in vivo imaging is used since its avoids the problem of dehalogenation of the ¹²⁵I or ¹³¹I-labeled monoclonal antibody by the liver. In addition, this radio nucleotide has a more favorable gamma emission energy for imaging (Perkins et al., Eur. J. Nucl. Med. 10:296-301 (1985); Carasquillo et al., J. Nucl. Med. 28:281-287 (1987)). For example, ¹¹¹In coupled to monoclonal antibodies with 1-(P-isothiocyanatobenzyl)-DPTA has shown little uptake in non-tumorous tissues, particularly the liver, and therefore enhances specificity of tumor localization (Esteban et al., J. NucL. Med. 28:861-870 (1987)).

Examples of suitable non-radioactive isotopic labels include ¹⁵⁷Gd, ⁵⁵Mn, ¹⁶²Dy, ⁵²Tr, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, an o-phthaldehyde label, and a fluorescamine label.

Examples of suitable toxin labels include diphtheria toxin, ricin, and cholera toxin.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and Fe.

Typical techniques for binding the above-described labels to antibodies are provided by Kennedy et al., Clin. Chim. Acta 70:1-31 (1976), and Schurs et al., Clin. Chim. Acta 81:1-40 (1977). Coupling techniques mentioned in the latter are the glutaraldehyde method, the periodate method, the dimaleimide method, the m-maleimidobenzyl-N-hydroxy-succinimide ester method, all of which methods are incorporated by reference herein.

The present invention also relates to vectors which include a nucleotide sequence encoding a compound of the present invention or parts thereof, host cells which are genetically engineered with the recombinant vectors, and the production of the compounds of the present invention or parts thereof by recombinant techniques.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E. coli lac, trp and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the mature transcripts expressed by the constructs will preferably include a translation initiating at the beginning and a termination codon (UAA, UGA or UAG) appropriately positioned at the end of the polypeptide to be translated.

As indicated, the expression vectors will preferably include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS and Bowes melanoma cells; and plant cells. Appropriate culture mediums and conditions for the above-described host cells are known in the art. For example, MHC Class I molecules can be expressed in Drosophila cells (U.S. Pat. No. 6,001,365).

Among vectors preferred for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pIRESbleo3, pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to solubilize proteins. For example, EP-A-O 464 533 (Canadian counterpart 2045869) discloses fusion proteins comprising various portions of constant region of immunoglobin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when the Fc portion proves to be a hindrance to use in therapy and diagnosis, for example when the fusion protein is to be used as an antigen for immunizations. In drug discovery, for example, human proteins, such as the hIL5-receptor, have been fused with Fc portions for the purpose of high-throughput screening assays to identify antagonists of hIL-5. See, D. Bennett et al., J. Mol. Recognition 8:52-58 (1995) and K. Johanson et al., J. of Biol. Chem. 270(16):9459-9471 (1995).

Several reports have described secretion and assembly of fusion proteins comprised of diverse sequences linked to the carboxyl terminus of immunoglobulin chains (Harvill, E. T. et al., J. Immunol. 157:3165-70 (1996); Shin, S. U. et al., J. Immunology 158: 4797-4804 (1997); Penichet, M. L. et al., J. Immunol. 163:4421-26 (1999); Zhang, H. F. et al., J. Clin. Invest 103:55-61 (1999)). Fusion proteins of the compounds of this invention will likewise retain amino terminal sequences of the immunoglobulin chain that direct secretion. MHC molecules linked to the carboxyl terminus of the immunoglobulin chains are stripped of hydrophobic transmembrane sequences and should not interfere with secretion.

The polypeptide can be recovered and purified from recombinant cell cultures by well-known methods including ammonium sulfate or ethanol precipitation, acid extraction, anion or cation exchange chromatography, phosphocellulose.chromatography, hydrophobic interaction chromatography, affinity chromatography, hydroxylapatite chromatography and lectin chromatography. Most preferably, high performance liquid chromatography (“HPLC”) is employed for purification. Polypeptides useful in the present invention include naturally purified products, products of chemical synthetic procedures, and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes.

The ability of a compound of the present invention to modulate an immune response can be readily determined by an in vitro assay. T cells for use in the assays include transformed T cell lines, such as T cell hybridomas, or T cells which are isolated from a mammal, e.g., from a human or from a rodent such as a mouse. T cells can be isolated from a mammal by known methods. See, for example, Shimonkevitz et al., J. Exp. Med. 158:303 (1983).

A suitable assay to determine if a compound of the present invention is capable of modulating the activity of T cells is conducted by coculturing T cells and antigen presenting cells, adding the particular compound of interest to the culture medium, and measuring IL-2 production. A decrease in IL-2 production over a standard indicates the compound can suppress an immune response. An increase in IL-2 production over a standard indicates the compound can stimulate an immune response.

The T cells employed in the assays are incubated under conditions suitable for proliferation. For example, a DO11.10 T cell hybridoma is suitably incubated at about 37° C. and 5% CO₂ in complete culture medium (RPMI 1640 supplemented with 10% FBS, penicillin/streptomycin, L-glutamine and 5×10⁻⁵ M 2-mercaptoethanol). Serial dilutions of the compound can be added to the T cell culture medium. Suitable concentrations of the compound added to the T cells typically will be in the range of from 10⁻¹² to 10⁻⁶ M. Use of antigen dose and APC numbers giving slightly submaximal T cell activation is preferred to detect inhibition of T cell responses by the compounds of the invention.

Alternatively, rather than measurement of an expressed protein such as IL-2, modulation of T cell activation can be suitably determined by changes in antigen-dependent T cell proliferation as measured by radiolabelling techniques as are recognized in the art. For example, a labeled (e.g., tritiated) nucleotide may be introduced to an assay culture medium. Incorporation of such a tagged nucleotide into DNA serves as a measure of T cell proliferation. This assay is not suitable for T cells that do not require antigen presentation for growth, e.g., T cell hybridomas. A difference in the level of T cell proliferation following contact with the compound of the invention indicates the complex modulates activity of the T cells. For example, a decrease in T cell proliferation indicates the compound can suppress an immune response. An increase in T cell proliferation indicates the compound can stimulate an immune response.

Additionally, the ⁵¹Cr release assay, described below, can be used to determine CTL activity.

These in vitro assays can be employed to select and identify peptide that are capable of modulating an immune response. Assays described above, e.g., measurement of IL-2 production or T cell proliferation, are employed to determine if contact with the compound modulates T cell activation.

In vivo assays also may be suitably employed to determine the ability of a compound of the invention to modulate the activity of T cells. For example, a compound of interest can be assayed for its ability to inhibit immunoglobulin class switching (i.e. IgM to IgG). See, e.g., Linsley et al., Science 257:792-795 (1992)). For example, a compound of the invention can be administered to a mammal such as a mouse, blood samples obtained from the mammal at the time of initial administration and several times periodically thereafter (e.g. at 2, 5 and 8 weeks after administration). Serum is collected from the blood samples and assayed for the presence of antibodies raised by the immunization. Antibody concentrations may be determined.

The present invention also includes pharmaceutical compositions comprising a compound described above in combination with a suitable pharmaceutical carrier. Such compositions comprise a therapeutically effective amount of the compound and a pharmaceutically acceptable carrier or excipient. Such a carrier includes but is not limited to saline, buffered saline, dextrose, water, glycerol, ethanol, and combinations thereof. The formulation should suit the mode of administration.

The present invention also includes a method of modulating, i.e., either stimulating or inhibiting an immune response, comprising administering to an animal and effective amount of a compound or composition of the invention.

The compounds of the present invention may be administered in pharmaceutical compositions in combination with one or more pharmaceutically acceptable excipients. It will be understood that, when administered to a human patient, the total daily usage of the pharmaceutical compositions of the present invention will be decided by the attending physician within the scope of sound medical judgment. The specific therapeutically effective dose level for any particular patient will depend upon a variety of factors including the type and degree of the response to be achieved; the specific composition of another agent, if any, employed; the age, body weight, general health, sex and diet of the patient; the time of administration, route of administration, and rate of excretion of the composition; the duration of the treatment; drugs (such as a chemotherapeutic agent) used in combination or coincidental with the specific composition; and like factors well known in the medical arts. Suitable formulations, known in the art, can be found in Remington's Pharmaceutical Sciences (latest edition), Mack Publishing Company, Easton, Pa.

The compound to be used in the therapy will be formulated and dosed in a fashion consistent with good medical practice, taking into account the clinical condition of the individual patient (especially the side effects of treatment with the compounds alone), the site of delivery of the compound, the method of administration, the scheduling of administration, and other factors known to practitioners. The “effective amount” of the compounds of the invention for purposes herein is thus determined by such considerations.

Pharmaceutical compositions of the invention may be administered orally, intravenously, rectally, parenterally, intracistemally, intradermally, intravaginally, intraperitoneally, topically (as by powders, ointments, gels, creams, drops or transdermal patch), bucally, or as an oral or nasal spray. The term “parenteral” as used herein refers to modes of administration which include intravenous, intramuscular, intraperitoneal, intrastemal, subcutaneous and intraarticular injection and infusion.

The pharmaceutical compositions are administered in an amount which is effective for treating and/or prophylaxis of the specific indication. In most cases, the dosage is from about 1 μg/kg to about 30 mg/kg body weight daily, taking into account the routes of administration, symptoms, etc. However, the dosage can be as low as 0.001 μg/kg.

As a general proposition, the total pharmaceutically effective amount of the compositions administered parenterally per dose will be in the range of about 1 μg/kg/day to 100 mg/kg/day of patient body weight, although, as noted above, this will be subject to therapeutic discretion. If given continuously, the composition is typically administered at a dose rate of about 1 μg/kg/hour to about 5 mg/kg/hour, either by 1-4 injections per day or by continuous subcutaneous infusions, for example, using a mini-pump. An intravenous bag solution or bottle solution may also be employed.

The compounds of the invention may also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release compositions also include liposomally entrapped compositions of the present invention. Liposomes are prepared by methods known per se: DE 3,218,121; Epstein, et al., Proc. NatL Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, in one embodiment, the composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compositions that are known to be deleterious to polypeptides.

Generally, the formulations are prepared by contacting the compounds of the invention uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes. Suitable formulations, known in the art, can be found in Remington's Pharmaceutical Sciences (latest edition), Mack Publishing Company, Easton, Pa.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; sugar alcohols such as mannitol or sorbitol; counterions such as sodium; and/or nonionic surfactants such as polysorbates, poloxamers, or PEG.

The compositions are typically formulated in such vehicles at a concentration of about 0.01 μg/ml to 100 mg/ml, preferably 0.01 μg/ml to10 mg/ml, at a pH of about 3 to 8. It will be understood that the use of certain of the foregoing excipients, carriers, or stabilizers will result in the formation of salts.

Compositions to be used for therapeutic administration must be sterile. Sterility is readily accomplished by filtration through sterile filtration membranes (e.g., 0.2 micron membranes). Therapeutic compositions generally are placed into a container having a sterile access port, for example, an intravenous solution bag or vial having a stopper pierceable by a hypodermic injection needle.

The compounds of the invention ordinarily will be stored in unit or multi-dose containers, for example, sealed ampules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized composition using bacteriostatic Water-for-Injection.

Dosaging may also be arranged in a patient specific manner to provide a predetermined concentration of activity in the blood, as determined by an RIA technique, for instance. Thus patient dosaging may be adjusted to achieve regular on-going trough blood levels, as measured by RIA, on the order of from 50 to 1000 ng/ml, preferably 150 to 500 ng/ml.

The compounds of the invention are useful for administration to any animal, preferably a mammal (such as apes, cows, horses, pigs, boars, sheep, rodents, goats, dogs, cats, chickens, monkeys, rabbits, ferrets, whales, and dolphins), and more preferably a human.

The invention also provides a pharmaceutical pack or kit comprising one or more containers filled with one or more of the ingredients of the pharmaceutical compositions of the invention. Associated with such containers can be a notice in the form prescribed by a governmental agency regulating the manufacture, use or sale of pharmaceuticals or biological products, which notice reflects approval by the agency of manufacture, use or sale for human administration. In addition, the compositions of the present invention may be employed in conjunction with other therapeutic compositions.

Other therapeutic compositions useful for administration along with a compound of the present invention include cytotoxic drugs, particularly those which are used for cancer therapy. Such drugs include, in general, alkylating agents, anti-proliferative agents, tubulin binding agents and the like. Preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin, aminopterin, methotrexate, methopterin, dichloromethotrexate, mitomycin C, porfiromycin, 5-fluorouracil, 6-mercaptopurine, cytosine arabinoside, podophyllotoxin, or podophyllotoxin derivatives such as etoposide or etoposide phosphate, melphalan, vinblastine, vincristine, leurosidine, vindesine, leurosine and the like. As noted previously, one skilled in the art may make chemical modifications to the desired compound in order to make reactions of that compound more convenient for purposes of preparing conjugates of the invention.

The compounds of the invention can be used to treat tumor-bearing animals, including humans, to generate an immune response against tumor cells. The generation of an adequate and appropriate immune response leads to tumor regression in vivo. Such “vaccines” can be used either alone or in combination with other therapeutic regimens, including but not limited to chemotherapy, radiation therapy, surgery, bone marrow transplantation, etc. for the treatment of tumors. For example, surgical or radiation techniques could be used to debulk the tumor mass, after which, the vaccine formulations of the invention can be administered to ensure the regression and prevent the progression of remaining tumor masses or micrometastases in the body. Alternatively, administration of the “vaccine” can precede such surgical, radiation or chemotherapeutic treatment.

Alternatively, the recombinant viruses of the invention can be used to immunize or “vaccinate” tumor-free subjects to prevent tumor formation. With the advent of genetic testing, it is now possible to predict a subject's predisposition for certain cancers. Such subjects, therefore, may be immunized using a compound comprising one or more antigenic peptides derived from tumors.

Suitable preparations of such vaccines include injectables, either as liquid solutions or suspensions; solid forms suitable for solution in, suspension in, liquid prior to injection, may also be prepared. The preparation may also be emulsified, or the polypeptides encapsulated in liposomes. The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine preparation may also include minor amounts of auxiliary substances such as wetting or emulsifying agents, pH buffering agents, and/or adjuvants which enhance the effectiveness of the vaccine.

Examples of adjuvants which may be effective, include, but are not limited to: aluminum hydroxide, N-acetyl-muramyl-L-threonyl-D-isoglutamine (thr-MDP), N-acetyl-nor-muramyl-L-alanyl-D-isoglutamine, N-acetylmuramyl-L-alanyl-D-isoglutaminyl-L-alanine-2-(1′-2′-dipalmitoyl-sn-glycero-3-hydroxyphosphoryloxy)-ethylamine, GM-CSF, QS-21 (investigational drug, Progenics Pharmaceuticals,Inc.), DETOX (investigational drug, Ribi Pharmaceuticals), BCG, and CpG rich oligonucleotides.

The composition, if desired, can also contain minor amounts of wetting or emulsifying agents, or pH buffering agents. The composition can be a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

Generally, the ingredients are supplied either separately or mixed together in unit dosage form, for example, as a dry lyophilized powder or water free concentrate in a hermetically sealed container such as an ampoule or sachette indicating the quantity of active agent. Where the composition is administered by injection, an ampoule of sterile diluent can be provided so that the ingredients may be mixed prior to administration.

In an alternate embodiment, compounds of the present invention may be used in adoptive immunotherapeutic methods for the activation of T lymphocytes that are histocompatible with the patient. (for methods of adoptive immunotherapy, see, e.g., Rosenberg, U.S. Pat. No. 4,690,915, issued Sep. 1, 1987; Zarling, et al., U.S. Pat. No. 5,081,029, issued Jan. 14, 1992). Such T lymphocytes may be isolated from the patient or a histocompatible donor. The T lymphocytes are activated in vitro by exposure to the compound of the invention. Activated T lymphocytes are expanded and inoculated into the patient in order to transfer T cell immunity directed against the particular antigenic peptide or peptides.

The compounds of the present invention may be administered along with other compounds which modulate an immune response, for example, cytokines.

The compounds of the invention may also be employed in accordance with the present invention by expression of such compounds, especially peptide-MHC Class I-antibody fusion compounds, in vivo, which is often referred to as “gene therapy.”

DNA that encodes a compound of this invention that is a direct fusion of antibody and MHC molecules may be introduced directly into cells by transfection or infection with a suitable vector so. as to give rise to synthesis and secretion of that compound by the successfully transfected or infected cells. However, since compounds of this invention require assembly of peptide-MHC Class I complexes and the desired peptides may not be present at high concentration in normal body cells, expression of compounds of the invention through DNA transfection or infection may require that DNA encoding the desired peptide be simultaneously introduced into the cell. This can be accomplished by cotransfection with separate DNA vector constructs or by co-expression in the same vector.

Thus, for example, cells from a patient may be engineered with a polynucleotide (DNA or RNA) encoding a compound of the invention ex vivo, with the engineered cells then being provided to a patient to be treated with the compounds. Such methods are well-known in the art. For example, cells may be engineered by procedures known in the art by use of a retroviral particle containing RNA encoding a compound of the present invention.

Similarly, cells may be engineered in vivo for expression of a compound in vivo by, for example, procedures known in the art. As known in the art, a producer cell for producing a retroviral particle containing RNA encoding the compound of the present invention may be administered to a patient for engineering cells in vivo and expression of the polypeptide in vivo. These and other methods for administering a polypeptide of the present invention by such method should be apparent to those skilled in the art from the teachings of the present invention. For example, the expression vehicle for engineering cells may be other than a retrovirus, for example, an adenovirus which may be used to engineer cells in vivo after combination with a suitable delivery vehicle. Examples of other delivery vehicles include an HSV-based vector system, adeno-associated virus vectors, pox viruses, and inert vehicles, for example, dextran coated ferrite particles.

Retroviruses from which the retroviral plasmid vectors hereinabove mentioned may be derived include, but are not limited to, lentiviruses, Moloney Murine Leukemia virus, spleen necrosis virus, retroviruses such as Rous Sarcoma Virus, Harvey Sarcoma virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The nucleic acid sequence encoding the compound of the present invention is under the control of a suitable promoter. Suitable promoters which may be employed include, but are not limited to, adenoviral promoters, such as the adenoviral major late promoter; or heterologous promoters, such as cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the β-actin promoter; and human growth hormone promoters.

The retroviral plasmid vector is employed to transduce packaging cell lines to form producer cell lines. Examples of packaging cell lines which may be transfected include, but are not limited to, the PE501, PA317, ψ-2, ψ-AM, PA12, T19-14x, VT-19-17-H2, ψCRE, ψCRIP, GP+E-86, GP+envAm12, and DAN cell lines as described in Miller, Human Gene Therapy 1:5-14 (1990), which is incorporated herein by reference in its entirety. The vector may transduce the packaging cells through any means known in the art. Such means include, but are not limited to, electroporation, the use of liposomes, and CaPO₄ precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

In certain embodiments, the polynucleotide constructs may be delivered as naked polynucleotides. By “naked” polynucleotides is meant that the polynucleotides are free from any delivery vehicle that acts to assist, promote, or facilitate entry into the cell, including viral sequences, viral particles, liposome formulation, lipofectin, precipitating agents and the like. Such methods are well known in the art and described, for example, in U.S. Pat. Nos. 5,593,972, 5,589,466, and 5,580,859.

The naked polynucleotides used in the invention can be those which do not integrate into the genome of the host cell. These may be non-replicating sequences, or specific replicating sequences genetically engineered to lack the genome-integration ability. Alternatively, the naked polynucleotides used in the invention may integrate into the genome of the host cell by, for example, homologous recombination, as discussed below. Preferably, the naked polynucleotide construct is contained in a plasmid. Suitable expression vectors for delivery include, but are not limited to, vectors such as pRSVcat (ATCC 37152), pSVL and MSG (Pharmacia, Uppsala, Sweden), pSV2dhfr (ATCC 37146) and pBC12MI (ATCC 67109). Additional suitable plasmids are discussed in more detail above.

The naked polynucleotides can be administered to any tissue (such as muscle tissue) or organ, as described above. In another embodiment, the naked polynucleotides are administered to the tissue surrounding the tissue of origin. In another embodiment, the naked polynucleotides are administered systemically, through intravenous injection.

For naked polynucleotide injection, an effective dosage amount of polynucleotide will be in the range of from about 0.05 μg/kg body weight to about 50 mg/kg body weight. Preferably, the dosage will be from about 0.005 mg/kg to about 20 mg/kg and more preferably from about 0.05 mg/kg to about 5 mg/kg. The appropriate and effective dosage of the polynucleotide construct can readily be determined by those of ordinary skill in the art and may depend on the condition being treated and the route of administration.

The constructs may also be delivered with delivery vehicles such as viral sequences, viral particles, liposome formulations, lipofectin, precipitating agents, etc. Such methods of delivery are known in the art. For example, the polynucleotide construct can be delivered specifically to hepatocytes through the method of Wu et al., J. Biol. Chem. 264:6985-16987 (1989).

In certain embodiments, the polynucleotide constructs are complexed in a liposome preparation. Liposomal preparations for use in the instant invention include cationic (positively charged), anionic (negatively charged) and neutral preparations. However, cationic liposomes are particularly preferred because a tight charge complex can be formed between the cationic liposome and the polyanionic nucleic acid. Cationic liposomes have been shown to mediate intracellular delivery of plasmid DNA (Felgner et al., Proc. Natl. Acad. Sci. USA (1987) 84:7413-7416); mRNA (Malone et al., Proc. Natl. Acad. Sci. USA (1989) 86:6077-6081); and purified transcription factors (Debs et al., J. Biol. Chem. (1990) 265:10189-10192), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See, also, Felgner et al., Proc. Natl Acad. Sci. USA (1987) 84:7413-7416). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 for a description of the synthesis of DOTAP (1,2-bis(oleoyloxy)-3-(trimethylammonio)propane) liposomes. Preparation of DOTMA liposomes is explained in the literature, see, e.g., P. Felgner et al., Proc. Natl. Acad. Sci. USA 84:7413-7417. Similar methods can be used to prepare liposomes from other cationic lipid materials.

Similarly, anionic and neutral liposomes are readily available, such as from Avanti Polar Lipids (Birmingham, Ala.), or can be easily prepared using readily available materials. Such materials include phosphatidyl, choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15° C. Alternatively, negatively charged vesicles can be prepared without sonication to produce multilamellar vesicles or by extrusion through nucleopore membranes to produce unilamellar vesicles of discrete size. Other methods are known and available to those of skill in the art.

The liposomes can comprise multilamellar vesicles (MLVs), small unilamellar vesicles (SUVs), or large unilamellar vesicles (LUVs), with SUVs being preferred. The various liposome-nucleic acid complexes are prepared using methods well known in the art. See, e.g., Straubinger et al., Methods of Immunology (1983), 101:512-527. For example, MLVs containing nucleic acid can be prepared by depositing a thin film of phospholipid on the walls of a glass tube and subsequently hydrating with a solution of the material to be encapsulated. SUVs are prepared by extended sonication of MLVs to produce a homogeneous population of unilamellar liposomes. The material to be entrapped is added to a suspension of preformed MLVs and then sonicated. When using liposomes containing cationic lipids, the dried lipid film is resuspended in an appropriate solution such as sterile water or an isotonic buffer solution such as 10 mM Tris/NaCl, sonicated, and then the preformed liposomes are mixed directly with the DNA. The liposome and DNA form a very stable complex due to binding of the positively charged liposomes to the cationic DNA. SUVs find use with small nucleic acid fragments. LUVs are prepared by a number of methods, well known in the art. Commonly used methods include Ca²⁺-EDTA chelation (Papahadjopoulos et al., Biochim. Biophys. Acta (1975) 394:483; Wilson et al., Cell (1979) 17:77); ether injection (Deamer, D. and Bangham, A., Biochim. Biophys. Acta (1976) 443:629; Ostro et al., Biochem. Biophys. Res. Commun. (1977) 76:836; Fraley et al., Proc. Natl. Acad. Sci. USA (1979) 76:3348); detergent dialysis (Enoch, H. and Strittmatter, P., Proc. Natl. Acad. Sci. USA (1979) 76:145); and reverse-phase evaporation (REV) (Fraley et al., J. Biol. Chem. (1980) 255:10431; Szoka, F. and Papahadjopoulos, D., Proc. Natl. Acad. Sci. USA (1978) 75:145; Schaefer-Ridder et al., Science (1982) 215:166).

Additional examples of useful cationic lipids include dipalmitoyl-phophatidylethanolamine 5-carboxyspen-nylamide (DPPES); 5-carboxyspermylglycine dioctadecylamide (DOGS); dimethyldioctdecyl-ammonium bromide (DDAB); and (±)-N,N-dimethyl-N-[2-(sperminecarboxamido)ethyl]-2,3-bis(dioleyloxy)-1-propaniminium pentahydrochloride (DOSPA). Non-diether cationic lipids, such as DL-1,2-dioleoyl-3-dimethylaminopropyl-β-hydroxyethylammonium (DORI diester), 1,2-O-dioleyl-3-dimethylaminopropyl-β-hydroxyethylammonium (DORIE diether), 1-O-oleyl-2-oleoyl-3-dimethylaminopropyl-β-hydroxyethylammonium (DORI ester/ether), and their salts promote in vivo gene delivery. Cationic cholesterol derivatives such as, {3β[N-N′,N′-dimethylamino)ethane]-carbomoyl}-cholesterol (DC-Chol), are also useful.

Preferred cationic lipids include: (±)-N-(2-hydroxyethyl)-N,N-dimethyl-2,3 -bis(tetradecyloxy)-1-propaniminium bromide; 3,5-(N,N-di-lysyl)diamino-benzoylglycyl-3-(DL-1,2-dioleoyl-dimethylaminopropyl-β-hydroxyethylamine) (DLYS-DABA-GLY-DORI diester); 3,5-(NN-dilysyl)-diaminobenzoyl-3-(DL-1,2-dioleoyl-dimethylaminopropyl-β-hydroxyethylamine) (DLYS-DABA-DORI diester); and 1,2-dioleoyl-sn-glycero-3-phosphoethanolamine. Also preferred is the combinations of the following lipids: (±)-N-(2-hydroxyethyl)-N,N-dimethyl-2,3-bis(tetradecyloxy)-1-propaniminium bromide and 1,2-dioleoyl-sn-glycero-3-phosphoethanolamine; and (±)-N-(2-hydroxyethyl)-N,N-dimethyl-2,3-bis(tetradecyloxy)-1-propaniminium bromide, and 1,2-dioleoyl-sn-glycero-3-phosphoethanolamine in a 1:1 ratio.

The lipid formulations may have a cationic lipid alone, or also include a neutral lipid such as cardiolipin, phosphatidylcholine, phosphatidylethanolamine, dioleoylphosphatylcholine, dioleoylphosphatidyl-ethanolamine, 1,2-dioleoyl-sn-glycero-3-phosphatidylethanolamine (DOPE), sphingomyelin, and mono-, di- or tri-acylglycerol).

Lipid formulations may also have cationic lipid together with a lysophosphatide. The lysophosphatide may have a neutral or a negative head group. Useful lysophosphatides include lysophosphatidylcholine, lysophosphatidyl-ethanolamine, and 1-oleoyl lysophosphatidylcholine. Lysophosphatide lipids are present Other additives, such as cholesterol, fatty acid, ganglioside, glycolipid, neobee, niosome, prostaglandin, sphingolipid, and any other natural or synthetic amphiphiles, can be used. A preferred molar ratio of cationic lipid to neutral lipid in these lipid formulations is from about 9:1 to about 1:9; an equimolar ratio is more preferred in the lipid-containing formulation in a 1:2 ratio of lysolipid to cationic lipid.

Generally, the ratio of DNA to liposomes will be from about 10:1 to about 1:10. Preferably, the ratio will be from about 5:1 to about 1:5. More preferably, the ratio will be about 3:1 to about 1:3. Still more preferably, the ratio will be about 1:1.

U.S. Pat. No. 5,676,954 reports on the injection of genetic material, complexed with cationic liposomes carriers, into mice. U.S. Pat. Nos. 4,897,355, 4,946,787, 5,049,386, 5,459,127, 5,589,466, 5,693,622, 5,580,859, 5,703,055, and international publication no. WO 94/9469 provide cationic lipids for use in transfecting DNA into cells and mammals. U.S. Pat. Nos. 5,589,466, 5,693,622, 5,580,859, 5,703,055, and international publication no. WO 94/9469 provide methods for delivering DNA-cationic lipid complexes to mammals.

In certain other embodiments, cells are engineered, ex vivo or in vivo, with the polynucleotide operably linked to a promoter contained in an adenovirus vector. Adenovirus can be manipulated such that it encodes and expresses the desired gene product, and at the same time is inactivated in terms of its ability to replicate in a normal lytic viral life cycle. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Suitable adenoviral vectors useful in the present invention are described, for example, in Kozarsky and Wilson, Curr. Opin. Genet. Devel. 3:499-503 (1993); Rosenfeld et al., Cell 68:143-155 (1992); Engelhardt et al., Human Genet. Ther. 4:759-769 (1993); Yang et al., Nature Genet. 7:362-369 (1994); Wilson et al., Nature 365:691-692 (1993); and U.S. Pat. No. 5,652,224, which are herein incorporated by reference. For example, the adenovirus vector Ad2 is useful and can be grown in human 293 cells. These cells contain the E1 region of adenovirus and constitutively express Ela and Elb, which complement the defective adenoviruses by providing the products of the genes deleted from the vector. In addition to Ad2, other varieties of adenovirus (e.g., Ad3, Ad5, and Ad7) are also useful in the present invention.

Preferably, the adenoviruses used in the present invention are replication deficient. Replication deficient adenoviruses require the aid of a helper virus and/or packaging cell line to. form infectious particles. The resulting virus is capable of infecting cells and can express a polynucleotide of interest which is operably linked to a promoter, for example, the polynucleotide of the present invention, but cannot replicate in most cells. Replication deficient adenoviruses may be deleted in one or more of all or a portion of the following genes: E1a, E1b, E3, E4, E2a, or L1 through L5.

In certain other embodiments, the cells are engineered, ex vivo or in vivo, using an adeno-associated virus (AAV). AAVs are naturally occurring defective viruses that require helper viruses to produce infectious particles (Muzyczka, N., Curr. Topics in Microbiol. Immunol. 158:97 (1992)). It is also one of the few viruses that may integrate its DNA into non-dividing cells. Vectors containing as little as 300 base pairs of AAV can be packaged and can integrate, but space for exogenous DNA is limited to about 4.5 kb. Methods for producing and using such AAVs are known in the art. See, for example, U.S. Pat. Nos. 5,139,941, 5,173,414, 5,354,678, 5,436,146, 5,474,935, 5,478,745, and 5,589,377.

For example, an appropriate AAV vector for use in the present invention will include all the sequences necessary for DNA replication, encapsidation, and host cell integration. The polynucleotide construct is inserted into the AAV vector using standard cloning methods, such as those found in Sambrook et al., Molecular Cloning: A Laboratory Manual, Cold Spring Harbor Press (1989). The recombinant AAV vector is then transfected into packaging cells which are infected with a helper virus, using any standard technique, including lipofection, electroporation, calcium phosphate precipitation, etc. Appropriate helper viruses include adenoviruses, cytomegaloviruses, vaccinia viruses, or herpes viruses. Once the packaging cells are transfected and infected, they will produce infectious AAV viral particles which contain the polynucleotide construct. These viral particles are then used to transduce eukaryotic cells, either ex vivo or in vivo. The transduced cells will contain the polynucleotide construct integrated into its genome, and will express the molecule of interest.

Any mode of administration of any of the above-described polynucleotides constructs can be used so long as the mode results in the expression of one or more molecules in an amount sufficient to provide a therapeutic effect. This includes direct needle injection, systemic injection, catheter infusion, biolistic injectors, particle accelerators (i.e., “gene guns”), gelfoam sponge depots, other commercially available depot materials, osmotic pumps (e.g., Alza minipumps), oral or suppositorial solid (tablet or pill) pharmaceutical formulations, and decanting or topical applications. For example, direct injection of naked calcium phosphate-precipitated plasmid into rat liver and rat spleen or a protein-coated plasmid into the portal vein has resulted in gene expression of the foreign gene in the rat livers (Kaneda et al., Science 243:375 (1989)).

A preferred method of local administration is by direct injection. Preferably, a recombinant molecule of the present invention complexed with a delivery vehicle is administered by direct injection into or locally within the area of the liver. Administration of a composition locally within the area of the liver refers to injecting the composition centimeters and preferably, millimeters within the liver.

Another method of local administration is to contact a polynucleotide-promoter construct of the present invention in or around a surgical wound. For example, a patient can undergo surgery and the polynucleotide construct can be coated on the surface of tissue inside the wound or the construct can be injected into areas of tissue inside the wound.

Therapeutic compositions useful in systemic administration, include recombinant molecules of the present invention complexed to a targeted delivery vehicle of the present invention. Suitable delivery vehicles for use with systemic administration comprise liposomes comprising ligands for targeting the vehicle to a particular site, for example, ligands for targeting the vehicle to a tissue of interest. Targeting vehicles for other tissues and organs are well known to skilled artisans.

Preferred methods of systemic administration, include intravenous injection, aerosol, oral and percutaneous (topical) delivery. Intravenous injections can be performed using methods standard in the art. Aerosol delivery can also be performed using methods standard in the art (see, for example, Stribling et al., Proc. Natl. Acad. Sci. USA 189:11277-11281, 1992, which is incorporated herein by reference). Oral delivery can be performed by complexing a polynucleotide construct of the present invention to a carrier capable of withstanding degradation by digestive enzymes in the gut of an animal. Examples of such carriers, include plastic capsules or tablets, such as those known in the art. Topical delivery can be performed by mixing a polynucleotide construct of the present invention with a lipophilic reagent (e.g., DMSO) that is capable of passing into the skin.

Determining an effective amount of substance to be delivered can depend upon a number of factors including, for example, the chemical structure and biological activity of the substance, the age and weight of the animal, the precise condition requiring treatment and its severity, and the route of administration. The frequency of treatments depends upon a number of factors, such as the amount of polynucleotide constructs administered per dose, as well as the health and history of the subject. The precise amount, number of doses, and timing of doses will be determined by the attending physician or veterinarian.

Direct administration of a DNA construct coding for a compound of the invention can be suitably accomplished for expression of the fusion compound within cells of the subject. Also, rather than directly administering nucleic acids coding for a compound of the invention to a subject, host compatible cells into which such nucleic acids have been introduced may be administered to the subject. Upon administration to a subject, such engineered cells can then express in vivo the compound of the invention. Such engineered cells can be administered to a subject to induce an immune response or alternatively to suppress an immune response, as disclosed herein.

A treatment method for suppression of an immune response provides for administration of a compound of the invention in which the peptide is a TCR antagonist or partial agonist. See Sette et al., Ann. Rev. Immunol. 12:413-431 (1994)). Peptides that are TCR antagonists or partial agonists can be readily identified and selected by the in vitro protocols identified above. A compound of the invention that contains a peptide that is a TCR antagonist or partial agonist is particularly preferred for treatment of allergies and autoimmune diseases.

Immunosuppressive therapies of the invention also may be used in combination as well as with other known immunosuppressive agents such as anti-inflammatory drugs to provide a more effective treatment of a T cell-mediated disorder. For example, other immunosuppressive agents useful in conjunction with the compounds of the invention include anti-inflammatory agents such as corticosteroids and nonsteroidal drugs.

The invention also provides methods for invoking an immune response in a mammal such as a human, including vaccinating a mammal with a compound or composition described herein.

The compounds of the invention are useful for raising an immune response and treating hyperproliferative disorders. Examples of hyperproliferative disorders that can be treated by the compounds of the invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital.

Similarly, other hyperproliferative disorders can also be treated by the compounds of the invention. Examples of such hyperproliferative disorders include, but are not limited to: hypergammaglobulinemia, lymphoproliferative disorders, paraproteinemias, purpura, sarcoidosis, Sezary Syndrome, Waldenstron's Macroglobulinemia, Gaucher's Disease, histiocytosis, and any other hyperproliferative disease, besides neoplasia, located in an organ system listed above.

The compounds of the present invention are also useful for raising an immune response against infectious agents. Viruses are one example of an infectious agent that can cause disease or symptoms that can be treated by the compounds of the invention. Examples of viruses, include, but are not limited to the following DNA and RNA viral families: Arbovirus, Adenoviridae, Arenaviridae, Arterivirus, Bimaviridae, Bunyaviridae, Caliciviridae, Circoviridae, Coronaviridae, Flaviviridae, Hepadnaviridae (hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picomaviridae, Poxviridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, measles, mumps, parainfluenza, rabies, the common cold, Polio, leukemia, Rubella, sexually transmitted diseases, skin diseases (e.g., Kaposi's, warts), and viremia.

Similarly, bacterial or fungal agents that can cause disease or symptoms and that can be treated by the compounds of the invention include, but are not limited to, the following Gram-Negative and Gram-positive bacterial families and fimgi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping. Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections.

Moreover, parasitic agents causing disease or symptoms that can be treated by the compounds of the invention include, but are not limited to, the following families: amebiasis, babesiosis, coccidiosis, cryptosporidiosis, dientamoebiasis, dourine, ectoparasitic, giardiasis, helminthiasis, leishmaniasis, theileriasis, toxoplasmosis, trypanosomiasis, and trichomonas.

Additionally, the compounds of the invention are useful for treating autoimmune diseases. An autoimmune disease is characterized by the attack by the immune system on the tissues of the victim. In autoimmune diseases, the recognition of tissues as “self” apparently does not occur, and the tissue of the afflicted subject is treated as an invader—i.e., the immune system sets about destroying this presumed foreign target. The compounds of the present invention are therefor useful for treating autoimmune diseases by desensitizing the immune system to these self antigens by provided a TCR signal to T cells without a costimulatory signal or with an inhibitory signal.

Examples of autoimmune diseases which may be treated using the compounds of the present invention include, but are not limited to Addison's Disease, hemolytic anemia, antiphospholipid syndrome, rheumatoid arthritis, dermatitis, allergic encephalomyelitis, glomerulonephritis, Goodpasture's Syndrome, Graves' Disease, multiple sclerosis, myasthenia gravis, neuritis, ophthalmia, bullous pemphigoid, pemphigus, polyendocrinopathies, purpura, Reiter's Disease, Stiff-Man Syndrome, autoimmune thyroiditis, systemic lupus erythematosus, autoimmune pulmonary inflammation, Guillain-Barre Syndrome, insulin dependent diabetes mellitis, autoimmune inflammatory eye disease, autoimmune hemolysis, psoriasis, juvenile diabetes, primary idiopathic myxedema, autoimmune asthma, scleroderma, chronic hepatitis, hypogonadism, pernicious anemia, vitiligo, alopecia areata, Coeliac disease, autoimmune enteropathy syndrome, idiopathic thrombocytic purpura, acquired splenic atrophy, idiopathic diabetes insipidus, infertility due to antispermatazoan antibodies, sudden hearing loss, sensoneural hearing loss, polymyositis, autoimmune demyelinating diseases, traverse myelitis, ataxic sclerosis, progressive systemic sclerosis, dermatomyositis, polyarteritis nodosa, idiopathic facial paralysis, cryoglobulinemia, inflammatory bowel diseases, Hashimoto's disease, adrenalitis, hypoparathyroidism, and ulcerative colitis.

Similarly, allergic reactions and conditions, such as asthma (particularly allergic asthma) or other respiratory problems, may also be treated by compounds of the invention. Moreover, the compounds of the invention can be used to treat anaphylaxis, hypersensitivity to an antigenic molecule, or blood group incompatibility.

The compounds of the invention may also be used to treat and/or prevent organ rejection or graft-versus-host disease (GVHD). Organ rejection occurs by host immune cell destruction of the transplanted tissue through an immune response. Similarly, an immune response is also involved in GVHD, but, in this case, the foreign transplanted immune cells destroy the host tissues. The administration of the compounds of the invention that inhibit an immune response may be an effective therapy in preventing organ rejection or GVHD.

The compounds of the invention which can inhibit an immune response are also useful for treating and/or preventing atherosclerosis; olitis; regional enteritis; adult respiratory distress syndrome; local manifestations of drug reactions, such as dermatitis, etc.; inflammation-associated or allergic reaction patterns of the skin; atopic dermatitis and infantile eczema; contact dermatitis; psoriasis; lichen planus; allergic enteropathies; allergic rhinitis; bronchial asthma; hypersensitivity or destructive responses to infectious agents; poststreptococcal diseases, e.g. cardiac manifestations of rheumatic fever, and the like.

Further, the compounds of the invention can be used as a male or female contraceptive. For example, a compound of the invention which is useful as a male contraceptive comprises as the antigenic peptide a peptide derived from PH30 beta chain sperm surface protein. See U.S. Pat. No. 5,935,578. A compound of the invention which is useful as a female contraceptive may comprise as the antigenic peptide a peptide derived from the human ZP2 or the human ZP3 protein. See U.S. Pat. No. 5,916,768.

A preferred method of delivering compounds of the invention is to administer them directly (iv, im, id, po) in the absence or presence of adjuvants such as oil and water emulsions, alum, CpG oligonucleotides, or cytokines such as GM-CSF. Another approach is to isolate patient PBL, purify PBMC and generate dendritic cells by a modification of the above protocol employing culture medium approved for clinical use such as X-VIVO or AIM-V and immunomagnetic bead separation of monocytes and lymphocytes rather than sheep erythrocyte rosetting (Romani, N., et al. J. Immunol. Methods. 196:137-151 (1996)). These cells can be pulsed in vitro with the compounds of the invention and then administered to the patient. This approach circumvents potential in vivo clearance of the compounds of the invention in the circulation, allows utilization of higher concentrations of the compound in vitro than would be possible or allowed in vivo, and ensures effective delivery of dendritic cells armed and ready to stimulate a primary T cell response. A secondary injection of pre-loaded DC or compound alone may be employed to boost the immune response. The magnitude of T cell responses induced is determined in vitro by a variety of assays for antigen-specific T cell activation as described herein or by staining with tetrameric complexes of the same peptide-MHC Class I ligand as described herein.

Having generally described the invention, the same will be more readily understood by reference to the following examples, which are provided by way of illustration and are not intended as limiting.

### EXAMPLES

### Example 1

### Chimeric β₂-microglobulin-F(ab) Fragment

In this example, a chimeric F(ab) fragment containing β₂-microglobulin coupled in frame with VH and CHi from IgG is made. Assembly takes place in a three-step process. In step A, custom PCR primers are used to amplify the β₂-microglobulin gene including the signal sequence. In step B, custom PCR primers are used to amplify the VH cloning cassette and the CH1 domain of IgG1. Finally, in step C all the components are assembled. Using the primers described, the β₂-microglobulin and IgG1 proteins are separated by a 12 amino acid linker.

Step A. Production of β₂-microglobulin

The fragment is generated by standard PCR using plasmid DNA as template and the following primers: (1) sense 5′-ATCGATATGTCTCGCTCCGTGGCCTTAGCT-3′ (SEQ ID NO:3) (ClaI restriction site is in bold); and (2) anti-sense 5′-CGGGGTACCTGACCCACCGCCTCCCATGTCTCGATCCCACTTAAC-3′ (SEQ ID NO:4) (linker is in bold; KpnI site is bolded and underlined). In a preferred embodiment, the template contains a three nucleotide mutation at position 222-224 of the β₂-microglobulin open reading frame. The net effect of this mutation is the substitution of a valine for serine at amino acid 74 of the β₂-microglobulin protein.

Step B. Production of the VH Cloning Cassette and the CHI Domain of IgG1

The fragment of interest is generated by standard PCR using plasmid DNA as template and the following primers: (3) Sense 5′-CGGGGTACCGGAGGCGGTGGGTCAGGCGCGCATATGGTCACC-3′ (SEQ ID NO:5) (linker is in bold; KpnI restriction site is bolded and underlined); and (4) Anti-sense 5-CGGGGATCCCTATTTCTTGTCCACCTTGGTGTT-3′ (SEQ ID NO:6) (BamHI site is bolded; stop codon is underlined).

Step C. Assembly of Chimeric β₂-microglobulin-F(ab) Fragment

Fragments A and B separately undergo PCR amplification and gel purification using standard conditions. Each fragment is digested with KpnI to generate overlapping sites for ligation. The fragments are then ligated at the KpnI site. The resulting product is then digested with ClaI and BamHI to create overlapping fragments for ligation into a mammalian expression construct. The complete gene is designed for insertion into the retroviral expression vector pIRESbleo3 (Clontech). However, this strategy is not limited to the use of pIRESbleo3. Specifically, the use of other expression vectors simply requires re-engineering of the restriction digestion sites flanking the complete construct (ClaI and BamHI). Nucleotide and protein sequence is presented without a v-gene. Any given v-gene can be inserted between the BssHII (bold) and BstEII (double underline, italics) sites.

The final sequence is: ATCGATATGTCTCGCTCCGTGGCCTT AGCTGTGCTCGCGCTACTCTCTCTTTCTGGCCTGGAGGCTATCCAGC GTACTCCAAAGATTCAGGTTTACTCACGTCATCCAGCAGAGAATGG AAAGTCAAATTTCCTGAATTGCTATGTGTCTGGGTTTCATCCATCCG ACATTGAAGTTGACTTACTGAAGAATGGAGAGAGAATTGAAAAAG TGGAGCATTCAGACTTGTCTTTCAGCAAGGACTGGTCTTTCTATCTC TTGTACTACACTGAATTCACCCCCACTGAAAAAGATGAGTATGCCT GCCGTGTGAACCATGTGACTTTGTCACAGCCCAAGATAGTTAAGTG GGATCGAGACATGGGAGGCGGTGGGTCAGGTACCGGAGGCGGTGG GTCAGGCGCGCATATGGTCACCGTCTCCTCAGCCTCCACCAAGGGC CCATCGGTCTTCCCCCTGGCACCCTCCTCCAAGAGCACCTCTGGGG GCACAGCGGCCCTGGGCTGCCTGGTCAAGGACTACTTCCCCGAACC GGTGACGGTGTCGTGGAACTCAGGCGCCCTGACCAGCGGCGTGCA CACCTTCCCGGCTGTCCTACAGTCCTCAGGACTCTACTCCCTCAGCA GCGTCGTGACCGTGCCCTCCAGCAGCTTGGGCACCCAGACCTACAT CTGCAACGTGAATCACAAGCCCAGCAACACCAAGGTGGACAAGAA ATAGGGATCCCCG-729 (SEQ ID NO:7) (double underline: ClaI restriction site; single underline: <2M Signal sequence; bold and underlined: linker; bold: BssHII restriction site; double underline, italics: BstEII restriction site; and italic underline: BamHII restriction site).

The resulting polypeptide sequence is: MSRSVALAVLALLSLSGLEAIQRTPKIQVYSRHPAENGKSNFLNCYVS GFHPSDIEVDLLKNGERIEKVEHSDLSFSKDWSFYLLYYTEFTPTEKDE YACRVNHVTLSQPKIVKWDRDMGGGGSGTGGGGSGAHMVTVSSAS TKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKK-237 (SEQ ID NO:8) (the linker is in bold and underlined). The variable gene sequence is introduced at the histidine in bold and the rest of the bolded amino acids are removed upon insertion of the variable gene sequence.

### Example 2

### Chimeric β₂ Microglobulin-F(ab′)2 Fragment

In this example, a chimeric F(ab′)2 fragment containing <2-microglobulin coupled in frame with VH, CH1, and the hinge region from IgG is created. Assembly takes place in a three-step process. In step A, custom PCR primers are used to amplify the <2-microglobulin gene including the signal sequence. In step B, custom PCR primers are used to amplify the VH cloning cassette, the CH1 domain, and the hinge region of IgG1 (or IgG3 for greater flexibility). Finally, in step C all components are assembled. Using the primers described, the <2-microglobulin and IgG1 proteins are separated by a 12 amino acid linker.

Step A. Production of <2-microglobulin

The fragment is generated as described above in Example 1.

Step B. Production of VH Cassette/CH1/Hinge Domain of IgG1

The fragment of interest is generated by standard PCR using plasmid DNA as template and the following primers: (3) Sense 5′-CGGGGTACCGGAGGCGGTGGGTCAGGCGCGCATATGGTCACC-3′ (SEQ ID NO:5) (linker is in bold; KpnI restriction site is bolded and underlined); and (5) anti-sense 5′-CGGGGATCCCTATGGGCACGGTGGGCATGTGTG-3′ (SEQ ID NO:9) (BamHI site is bolded; stop codon is underlined). In other embodiments, the CH1 and hinge region derives from other immunoglobulin isotypes, including IgG2, IgG3, IgG4, IgA, IgM, IgD or IgE. Particularly preferred is the longer and more flexible IgG3 hinge region.

Step C. Assembly of Chimeric <2-microglobulin-F(ab′)2 Fragment

Fragments A and B separately undergo PCR amplification and gel purification using standard conditions. Each fragment is digested with KpnI to generate overlapping sites for ligation. The fragments are then ligated at the KpnI site. The resulting product is then digested with ClaI and BamHI to create overlapping fragments for ligation into a mammalian expression construct. The complete gene is designed for insertion into the retroviral expression vector pIRESbleo3 (Clontech). However, this strategy is not limited to the use of pIRESbleo3. Specifically, the use of other expression vectors simply requires re-engineering of the restriction digestion sites flanking the complete construct (ClaI and BamHI). Nucleotide and protein sequence is presented without a v-gene. Any given v-gene can be inserted between the BssHII (bold) and BstEII (double underline, italics) sites.

The final sequence is: ATCGATATGTCTCGCTCCGTGGC CTTAGCTGTGCTCGCGCTACTCTCTCTTTCTGGCCTGGAGGCTATCC AGCGTACTCCAAAGATTCAGGTTTACTCACGTCATCCAGCAGAGAA TGGAAAGTCAAATTTCCTGAATTGCTATGTGTCTGGGTTTCATCCAT CCGACATTGAAGTTGACTTACTGAAGAATGGAGAGAGAATTGAAA AAGTGGAGCATTCAGACTTGTCTTTCAGCAAGGACTGGTCTTTCTA TCTCTTGTACTACACTGAATTCACCCCCACTGAAAAAGATGAGTAT GCCTGCCGTGTGAACCATGTGACTTTGTCACAGCCCAAGATAGTTA AGTGGGATCGAGACATGGGAGGCGGTGGGTCAGGTACCGGAGGCG GTGGGTCAGGCGCGCATATGTCACCGTCTCCTCAGCCTCCACCAAG GGCCCATCGGTCTTCCCCCTGGCACCCTCCTCCAAGAGCACCTCTG GGGGCACAGCGGCCCTGGGCTGCCTGGTCAAGGACTACTTCCCCGA ACCGGTGACGGTGTCGTGGAACTCAGGCGCCCTGACCAGCGGCGT GCACACCTTCCCGGCTGTCCTACAGTCCTCAGGACTCTACTCCCTCA GCAGCGTCGTGACCGTGCCCTCCAGCAGCTTGGGCACCCAGACCTA CATCTGCAACGTGAATCACAAGCCCAGCAACACCAAGGTGGACAA GAAAGTTGAGCCCAAATCTTGTGACAAAACTCACACATGCCCACCG TGCCCATAGGGATCCCCG-777 (SEQ ID NO:10) (double underline: ClaI restriction site; single underline: <2M signal sequence; bold and underlined: linker; bold: BssHII restriction site; double underline, italics: BstEII restriction site; italic underline: BamHI restriction site).

The encoded polypeptide sequence is: MSRSVALAVLALLSLSGLEAIQRTPKIQVYSRHPAENGKSNFLNCYVS GFHPSDIEVDLLKNGERIEKVEHSDLSFSKDWSFYLLYYTEFTPTEKDE YACRVNHVTLSQPKIVKWDRDMGGGGSGTGGGGSGAHMVTVSSAS TKGPSVFPLAPSSKSTSGGTAALGCLVKDYFPEPVTVSWNSGALTSGV HTFPAVLQSSGLYSLSSVVTVPSSSLGTQTYICNVNHKPSNTKVDKKV EPKSCDKTHTCPPCP-253 (SEQ ID NO:11) (the linker is in bold and underlined). The variable gene sequence is introduced at the histidine in bold and the rest of the bolded amino acids are removed upon insertion of the variable gene sequence.

### Example 3

### Chimeric <2 Microglobulin-full IgG1

The intent is to create a chimeric <2-microglobulin coupled in frame with the full IgG1. Assembly takes place in a three-step process. In step A, custom PCR primers are used to amplify the <2-microglobulin gene including the signal sequence. In step B custom PCR primers are used to amplify the full IgG1. Finally, in step C all components are assembled. Using the primers described, the <2-microglobulin and IgG1 are separated by a 12 amino acid linker.

Step A. Production of <2-microglobulin

The fragment is generated as described above in Example 1.

Step B. Production of IgG1

The fragment of interest is generated by standard PCR using plasmid DNA as template and the following primers: (3) Sense 5′-CGGGGTACCGGAGGCGGTGGGTCA GGCGCGCATATGGTCACC-3′ (SEQ ID NO:5) (linker is in bold; KpnI restriction site is bolded and underlined; and (6) Anti-sense 5′-CGGGGATCCCTATTTACCCGGAGACAGGGAGAG-3′ (SEQ ID NO:12) (BamHI site is bolded; stop codon is underlined). In other embodiments, the constant region derives from other immunoglobulin isotypes, including IgG2, IgG3, IgG4, IgA, IgM, IgD or IgE. Particularly preferred is IgG3 with its longer and more flexible hinge region.

Step C. Assembly of Chimeric <2-microglobulin-Full IgG1

Fragments A and B separately undergo PCR amplification and gel purification using standard conditions. Each fragment is digested with KpnI to generate overlapping sites for ligation. The fragments are then ligated at the KpnI site. The resulting product is then digested with ClaI and BamHI to create overlapping fragments for ligation into a mammalian expression construct. The complete gene is designed for insertion into the retroviral expression vector pIRESbleo3 (Clontech). However, this strategy is not limited to the use of pIRESbleo3. Specifically, the use of other expression vectors simply requires re-engineering of the restriction digestion sites flanking the complete construct (ClaI and BamHI). Nucleotide and protein sequence is presented without a v-gene. Any given v-gene can be inserted between the BssHII (bold) and BstEII (double underline, italics) sites.

The final sequence is: ATCGATATGTCTCGCTCCGTGGC CTTAGCTGTGCTCGCGCTACTCTCTCTTTCTGGCCTGGAGGCTATCC AGCGTACTCCAAAGATTCAGGTTTACTCACGTCATCCAGCAGAGAA TGGAAAGTCAAATTTCCTGAATTGCTATGTGTCTGGGTTTCATCCAT CCGACATTGAAGTTGACTTACTGAAGAATGGAGAGAGAATTGAAA AAGTGGAGCATTCAGACTTGTCTTTCAGCAAGGACTGGTCTTTCTA TCTCTTGTACTACACTGAATTCACCCCCACTGAAAAAGATGAGTAT GCCTGCCGTGTGAACCATGTGACTTTGTCACAGCCCAAGATAGTTA AGTGGGATCGAGACATGGGAGGCGGTGGGTCAGGTACCGGAGG CGGTGGGTCAGGCGCGCATATGGTCACCGTCTCCTCAGCCTCCAC CAAGGGCCCATCGGTCTTCCCCCTGGCACCCTCCTCCAAGAGCACC TCTGGGGGCACAGCGGCCCTGGGCTGCCTGGTCAAGGACTACTTCC CCGAACCGGTGACGGTGTCGTGGAACTCAGGCGCCCTGACCAGCG GCGTGCACACCTTCCCGGCTGTCCTACAGTCCTCAGGACTCTACTC CCTCAGCAGCGTCGTGACCGTGCCCTCCAGCAGCTTGGGCACCCAG ACCTACATCTGCAACGTGAATCACAAGCCCAGCAACACCAAGGTG GACAAGAAAGTTGAGCCCAAATCTTGTGACAAAACTCACACATGC CCACCGTGCCCAGCACCTGAACTCCTGGGGGGACCGTCAGTCTTCC TCTTCCCCCCAAAACCCAAGGACACCCTCATGATCTCCCGGACCCC TGAGGTCACATGCGTGGTGGTGGACGTGAGCCACGAAGACCCTGA GGTCAAGTTCAACTGGTACGTGGACGGCGTGGAGGTGCATAATGC CAAGACAAAGCCGCGGGAGGAGCAGTACAACAGCACGTACCGTGT GGTCAGCGTCCTCACCGTCCTGCACCAGGACTGGCTGAATGGCAAG GAGTACAAGTGCAAGGTCTCCAACAAAGCCCTCCCAGCCCCCATCG AGAAAACCATCTCCAAAGCCAAAGGGCAGCCCCGAGAACCACAGG TGTACACCCTGCCCCCATCCCGGGATGAGCTGACCAAGAACCAGGT CAGCCTGACCTGCCTGGTCAAAGGCTTCTATCCCAGCGACATCGCC GTGGAGTGGGAGAGCAATGGGCAGCCGGAGAACAACTACAAGACC ACGCCTCCCGTGCTGGACTCCGACGGCTCCTTCTTCCTCTACAGCA AGCTCACCGTGGACAAGAGCAGGTGGCAGCAGGGGAACGTCTTCT CATGCTCCGTGATGCATGAGGCTCTGCACAACCACTACACGCAGAA GAGCCTCTCC CTGTCTCCGGGTAAATAGGGATCCCCG-1428 (SEQ ID NO:13) (double underline: ClaI restriction site; single underline: <2M Signal sequence; bold and underlined: linker; bold: BssHII restriction site; double underline italics: BstEII restriction site; and italic underline: BamHI restriction site).

The resultant polypeptide sequence is: MSRSVALAVLALLSL SGLEAIQRTPKIQVYSRHPAENGKSNFLNCYVSGFHPSDIEVDLLKNGE RIEKVEHSDLSFSKDWSFYLLYYTEFTPTEKDEYACRVNHVTLSQPKIV KWDRDMGGGGSGTGGGGSGAHMVTVSSASTKGPSVFPLAPSSKSTS GGTAALGCLVKDYFPEPVTVSWNSGALTSGVHTFPAVLQSSGLYSLSS VVTVPSSSLGTQTYICNVNHKPSNTKVDKKVEPKSCDKTHTCPPCPAP ELLGGPSVFLFPPKPKDTLMISRTPEVTCVVVDVSHEDPEVKFNWYVD GVEVHNAKTKPREEQYNSTYRVVSVLTVLHQDWLNGKEYKCKVSNK ALPAPIEKTISKAKGQPREPQVYTLPPSRDELTKNQVSLTCLVKGFYPS DIAVEWESNGQPENNYKTTPPVLDSDGSFFLYSKLTVDKSRWQQGNV FSCSVMHEALHNHYTQKSLSLSPGK-470 (SEQ ID NO:14) (the linker is bold and underlined). The variable gene sequence is introduced at the histidine in bold and the rest of the bolded amino acids are removed upon insertion of the variable gene sequence.

### Example 4

### Chimeric F(ab)-<2-microglobulin

The intent is to create a chimeric F(ab) fragment containing VH and CH1 from IgG coupled in frame with <2-microglobulin. Assembly takes place in a three-step process. In step one, PCR is used to create the CH1 region preceded by a signal sequence (SS) for secretion and the cloning cassette for VH and followed by a linker with an embedded KpnI restriction site. In step two PCR is used to amplify the <2-microglobulin gene. Finally, in step three, restriction digestion at the KpnI site is used followed by ligation to combine the two fragments. The IgG and <2-microglobulin proteins are separated by a 12 amino acid linker.

Step A. Production of CH1/VH Cassette

Standard PCR is used to amplify CH1 with a pre-configured VH insertion site from a previously described template. Specifically, an IgG1 construct has been generated that allows for insertion of a variable gene of interest through BssHI and BstEII restriction sites. This construct is described elsewhere (U.S. Appl. Publ. No. 2002/0123057) and is available as template for PCR using the following primers: sense 5′-AATTGCGGCCGCAAACCATGGGATGGAGCTGTATCATC 3″ (SEQ ID NO: 15) (NotI and NcoI sites in bold); and anti-sense 5′-CGGGGTACCTGACCCACCGCCTCCTTTCTTGTCCACCTTGGTGTT 3′ (SEQ ID NO: 16) (linker is in bold; KpnI site is bolded and underlined). The PCR product is gel purified according to standard procedure.

Step B. Production of <2-microglobulin

The <2-microglobulin gene may be amplified to encode either the mature polypeptide, or it may include the 20-amino acid signal sequence. The sequence encoding the mature fragment is generated by standard PCR using plasmid DNA as template and the following primers: sense 5′ CGGGGTACCG GAGGCGGTGG GTCAATCCAG CGTACTCCA-3′ (SEQ ID NO:28) (linker is in bold; KpnI restriction site is bolded and underlined); and anti-sense 5′-CGGGATCCTT ACATGTCTCG ATCCCACTT-3′ (SEQ ID NO: 18) (BamHI restriction site is in bold). The sequence encoding the fragment which includes the 20-amino acid signal sequence is generated by standard PCR using plasmid DNA as template and the following primers: sense 5′-CGGGTACCGG AGGCGGTGGG TCAATGTCTC GCTCCGTG-3′ (SEQ ID NO:17) (linker is in bold; KpnI restriction site is bolded and underlined); and anti-sense 5′-CGGGATCCTT ACATGTCTCG ATCCCACTT-3′ (SEQ ID NO: 18) (BamHI restriction site is in bold.). The PCR product is gel purified according to standard procedure. In a preferred embodiment, the template contains a three nucleotide mutation at position 222-224 of the <2-microglobulin open reading frame which results in the substitution of a valine for serine at amino acid 74 of the full length <2-microglobulin protein (V74S) (or position 55 of the mature <2-microglobulin).

Step C. Assembled Chimeric F(ab)-<2-microglobulin Product

The above fragments “A” and “B” are joined by restriction digestion at the KpnI site followed by ligation employing standard protocols. The complete gene is designed for insertion into the expression vector pIRESbleo3 (Clontech). This strategy is not limited to the use of pIRESbleo3. Specifically, the use of other expression vector simply requires re-engineering of the restriction digestion sites flanking the complete construct (NotI and BamHI). Nucleotide and protein sequence is presented without a VH-gene. Any given VH-gene can be inserted between the BssHII (bold) and BstEII (double underline italics) sites. The resulting nucleotide sequence encoding the chimeric F(ab)-mature <2-microglobulin product is as follows:

(SEQ ID NO:29)

GCGGCCGCAAACCATGGGATGGAGCTGTATCATCCTCTTCTTGGTA
GCAACAGCTACAGGCGCGCATATGGTCACCGTCTCCTCAGCCTCCA
CCAAGGGCCCATCGGTCTTCCCCCTGGCACCCTCCTCCAAGAGCAC
CTCTGGGGGCACAGCGGCCCTGGGCTGCCTGGTCAAGGACTACTTC
CCCGAACCGGTGACGGTGTCGTGGAACTCAGGCGCCCTGACCAGC
GGCGTGCACACCTTCCCGGCTGTCCTACAGTCCTCAGGACTCTACT
CCCTCAGCAGCGTCGTGACCGTGCCCTCCAGCAGCTTGGGCACCCA
GACCTACATCTGCAACGTGKATCACAAGCCCAGCAACACCAAGGT
GGACAAGAAAGGAGGCGGTGGGTCAGGTACCGGAGGCGGTGG
GTCAATCCAGCGTACTCCAAAGATTCAGGTTTACTCACGTCATCCA
GCAGAGAATGGAAAGTCAAATTTCCTGAATTGCTATGTGTCTGGGT
TTCATCCATCCGACATTGAAGTTGACTTACTGAAGAATGGAGAGAG
AATTGAAAAAGTGGAGCATTCAGACTTGGTGTTCAGCAAGGACTG
GTCTTTCTATCTCTTGTACTACACTGAATTCACCCCCACTGAAAAAG
ATGAGTATGCCTGCCGTGTGAACCATGTGACTTTGTCACAGCCCAA
GATAGTTAAGTGGGATCGAGACATGTAAGGATCCCG - 720

Double underline: NotI restriction site; single underline: signal sequence; bold: BssHII restriction site; double underline italics: BstEII restriction site; bold and underlined: linker; single underline italics: BamHI restriction site.

The polypeptide sequence of the chimeric F(ab)-mature <2-microglobulin product is:

(SEQ ID NO:30)

MGWSCIILFLVATATGAHMVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKGGGGSGTGGGGSIQRTPKIQVY
SRHPAENGKSNFLNCYVSGFHPSDIEVDLLKNGERIEKVEHSDLVFSK
DWSFYLLYYTEFTPTEKDEYACRVNHVTLSQPKIVKWDRDM - 232.

The variable gene sequence is introduced at the histidine in bold and the rest of the bolded amino acids are removed upon insertion of the variable gene sequence. The linker is bolded and underlined.

The resulting nucleotide sequence encoding the chimeric F(ab)-<2-microglobulin product which retains the <2-microglobulin signal sequence is as follows:

(SEQ ID NO:19)

GCGGCCGCAAACCATGGGATGGAGCTGTATCATCCTCTTCTTGGTA
GCAACAGCTACAGGCGCGCATATGGTCACCGTCTCCTCAGCCTCCA
CCAAGGGCCCATCGGTCTTCCCCCTGGCACCCTCCTCCAAGAGCAC
CTCTGGGGGCACAGCGGCCCTGGGCTGCCTGGTCAAGGACTACTTC
CCCGAACCGGTGACGGTGTCGTGGAACTCAGGCGCCCTGACCAGC
GGCGTGCACACCTTCCCGGCTGTCCTACAGTCCTCAGGACTCTACT
CCCTCAGCAGCGTCGTGACCGTGCCCTCCAGCAGCTTGGGCACCCA
GACCTACATCTGCAACGTGAATCACAAGCCCAGCAACACCAAGGT
GGACAAGAAAGGAGGCGGTGGGTCAGGTACCGGAGGCGGTGG
GTCAATGTCTCGCTCCGTGGCCTTAGCTGTGCTCGCGCTACTCTCTC
TTTCTGGCCTGGAGGCTATCCAGCGTACTCCAAAGATTCAGGTTTA
CTCACGTCATCCAGCAGAGAATGGAAAGTCAAATTTCCTGAATTGC
TATGTGTCTGGGTTTCATCCATCCGACATTGAAGTTGACTTACTGAA
GAATGGAGAGAGAATTGAAAAAGTGGAGCATTCAGACTTGGTGTT
CAGCAAGGACTGGTCTTTCTATCTCTTGTACTACACTGAATTCACCC
CCACTGAAAAAGATGAGTATGCCTGCCGTGTGAACCATGTGACTTT
GTCACAGCCCAAGATAGTTAAGTGGGATCGAGACATGTAAGGATCC
CG - 780.

Double underline: NotI restriction site; single underline: signal sequence; bold: BssHII restriction site; double underline italics: BstEII restriction site; bold and underlined: linker; single underline italics: BamHI restriction site.

The polypeptide sequence of the chimeric F(ab)-<2-microglobulin product which retains the <2-microglobulin signal sequence is:

(SEQ ID NO:20)

MGWSCIILFLVATATGAHMVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKGGGGSGTGGGGSMSRSVALAV
LALLSLSGLEAIQRTPKIQVYSRHPAENGKSNFLNCYVSGFHPSDIEVD
LLKNGERIEKVEHSDLVFSKDWSFYLLYYTEFTPTEKDEYACRVNHVT
LSQPKIVKWDRDM - 252.

The variable gene sequence is introduced at the histidine in bold and the rest of the bolded amino acids are removed upon insertion of the variable gene sequence. The linker is bolded and underlined.

### Example 5

### Chimeric F(ab′)₂-<2 microglobulin

The intent is to create a chimeric F(ab′)₂ fragment containing VH,CH1, and the hinge region from IgG1 coupled in frame with <2-microglobulin. Assembly takes place in a three-step process. In step one, PCR is used to create the cloning cassette for VH, including the CH1 region, and the hinge region preceded by a signal sequence (SS) for secretion and followed by a linker with an embedded KpnI restriction site. In step two, PCR is used to amplify the <2-microglobulin gene. Finally, in step three, restriction digestion at the KpnI site is used followed by ligation to combine the two fragments. The IgG1 and <2-microglobulin proteins are separated by a 12 amino acid linker.

Step A. Production of VH Cassette/CH1/hinge

Standard PCR is used to amplify VH/CH1/hinge from template. Specifically, an IgG1 construct has been generated that allows for insertion of a variable gene of interest through BssHI and BstEII restriction sites. This construct is described elsewhere (U.S. Appl. Publ. No. 2002/0123057) and is available as template for PCR using the following primers: sense 5′ AATTGCGGCCGCAAACCATGG GATGGAGCTGTATCATC 3′ (SEQ ID NO:15) (Notl and NcoI sites in bold); and (8) anti-sense 5′ CGGGGTACCTGACCCACCGCCTCCTGGGCACGGTGGGCATGTGT G 3′ (SEQ ID NO:21) (linker is in bold; KpnI site is bolded and underlined). The PCR product is gel purified according to standard procedure. In other embodiments, the CH1 and hinge region derives from other immunoglobulin isotypes, including IgG2, IgG3, IgG4, IgA, IgM, IgD or IgE. Particularly preferred is the longer and more flexible IgG3 hinge region.

Step B

The DNA fragment encoding either mature <2-microglobulin, or <2-microglobulin and its 20-amino acid signal sequence, is generated as described in Example 4.

Step C. Assembled Chimeric F(ab′)₂-<2 Microglobulin.

The above fragments “A” and “B” are joined by restriction digestion at the KpnI site followed by ligation employing standard protocols. The complete gene is designed for insertion into the expression vector pIRESbleo3 (Clontech). This strategy is not limited to the use of pIRESbleo3. Specifically, the use of other expression vector simply requires re-engineering of the restriction digestion sites flanking the complete construct (NotI and BamHI). Nucleotide and protein sequence is presented without a VH-gene. Any given VH-gene can be inserted between the BssHII (bold) and BstEII (double underline italics) sites.

The resulting nucleotide sequence encoding the chimeric F(ab′)₂-mature <2-microglobulin product is as follows:

(SEQ ID NO:31)

GCGGCCGCAAACCATGGGATGGAGCTGTATCATCCTCTTCTTGGTA
GCAACAGCTACAGGCGCGCATATGGTCACCGTCTCCTCAGCCTCCA
CCAAGGGCCCATCGGTCTTCCCCCTGGCACCCTCCTCCAAGAGCAC
CTCTGGGGGCACAGCGGCCCTGGGCTGCCTGGTCAAGGACTACTTC
CCCGAACCGGTGACGGTGTCGTGGAACTCAGGCGCCCTGACCAGC
GGCGTGCACACCTTCCCGGCTGTCCTACAGTCCTCAGGACTCTACT
CCCTCAGCAGCGTCGTGACCGTGCCCTCCAGCAGCTTGGGCACCCA
GACCTACATCTGCAACGTGAATCACAAGCCCAGCAACACCAAGGT
GGACAAGAAAGTTGAGCCCAAATCTTGTGACAAAACTCACACATG
CCCACCGTGCCCAGGAGGCGGTGGGTCAGGTACCGGAGGCGGT
GGGTCAATCCAGCGTACTCCAAAGATTCAGGTTTACTCACGTCATC
CAGCAGAGAATGGAAAGTCAAATTTCCTGAATTGCTATGTGTCTGG
GTTTCATCCATCCGACATTGAAGTTGACTTACTGAAGAATGGAGAG
AGAATTGAAAAAGTGGAGCATTCAGACTTGGTGTTCAGCAAGGAC
TGGTCTTTCTATCTCTTGTACTACACTGAATTCACCCCCACTGAAAA
AGATGAGTATGCCTGCCGTGTGAACCATGTGACTTTGTCACAGCCC
AAGATAGTTAAGTGGGATCGAGACATGTAAGGATCCCG - 768.

Double underline: NotI restriction site; single underline: signal sequence; bold: BssHII restriction site; dashed underline: BstEII restriction site; bold and underlined: linker; single underline italics: BamHI restriction site.

The polypeptide sequence of the chimeric F(ab′)₂-mature <2-microglobulin product is:

(SEQ ID NO:32)

MGWSCIILFLVATATGAHMVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPGGGGSG
TGGGGSIQRTPKIQVYSRHPAENGKSNFLNCYVSGFHPSDIEVDLLKN
GERIEKVEHSDLVFSKDWSFYLLYYTEFTPTEKDEYACRVNHVTLSQP
KIVKWDRDM - 248.

The variable gene sequence is introduced at the histidine in bold and the rest of the bolded amino acids are removed upon insertion of the variable gene sequence. The linker is bolded and underlined.

The resulting nucleotide sequence encoding the chimeric F(ab′)₂-<2-microglobulin product which retains the <2-microglobulin signal sequence is as follows:

(SEQ ID NO:22)

GCGGCCGCAAACCATGGGATGGAGCTGTATCATCCTCTTCTTGGTA
GCAACAGCTACAGGCGCGCATATGGTCACCGTCTCCTCAGCCTCCA
CCAAGGGCCCATCGGTCTTCCCCCTGGCACCCTCCTCCAAGAGCAC
CTCTGGGGGCACAGCGGCCCTGGGCTGCCTGGTCAAGGACTACTTC
CCCGAACCGGTGACGGTGTCGTGGAACTCAGGCGCCCTGACCAGC
GGCGTGCACACCTTCCCGGCTGTCCTACAGTCCTCAGGACTCTACT
CCCTCAGCAGCGTCGTGACCGTGCCCTCCAGCAGCTTGGGCACCCA
GACCTACATCTGCAACGTGAATCACAAGCCCAGCAACACCAAGGT
GGACAAGAAAGTTGAGCCCAAATCTTGTGACAAAACTCACACATG
CCCACCGTGCCCAGGAGGCGGTGGGTCAGGTACCGGAGGCGGT
GGGTCAATGTCTCGCTCCGTGGCCTTAGCTGTGCTCGCGCTACTCT
CTCTTTCTGGCCTGGAGGCTATCCAGCGTACTCCAAAGATTCAGGT
TTACTCACGTCATCCAGCAGAGAATGGAAAGTCAAATTTCCTGAAT
TGCTATGTGTCTGGGTTTCATCCATCCGACATTGAAGTTGACTTACT
GAAGAATGGAGAGAGAATTGAAAAAGTGGAGCATTCAGACTTGGT
GTTCAGCAAGGACTGGTCTTTCTATCTCTTGTACTACACTGAATTCA
CCCCCACTGAAAAAGATGAGTATGCCTGCCGTGTGAACCATGTGAC
TTTGTCACAGCCCAAGATAGTTAAGTGGGATCGAGACATGTAAGGA
TCCCG - 828.

Double underline: NotI restriction site; single underline: signal sequence; bold: BssHII restriction site; dashed underline: BstEII restriction site; bold and underlined: linker; single underline italics: BamHI restriction site.

The polypeptide sequence of the chimeric F(ab′)₂-<2-microglobulin product which retains the <2-microglobulin signal sequence is:

(SEQ ID NO:23)

MGWSCIILFLVATATGAHMVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPGGGGSG
TGGGGSMSRSVALAVLALLSLSGLEAIQRTPKIQVYSRHPAENGKSNF
LNCYVSGFHPSDIEVDLLKNGERIEKVEHSDLVFSKDWSFYLLYYTEF
TPTEKDEYACRVNHVTLSQPKIVKWDRDM - 268.

The variable gene sequence is introduced at the histidine in bold and the rest of the bolded amino acids are removed upon insertion of the variable gene sequence. The linker is bolded and underlined.

### Example 6

### Chimeric Full IgG1-<2 Microglobulin

The intent is to create a complete immunoglobulin IgG1 containing VH, CH1, Hinge region, CH2, and CH3 coupled in frame with <2-microglobulin. Assembly takes place in a three-step process. In step one, PCR is used to create the cloning cassette for VH and the full IgG1 heavy chain constant region preceded by a signal sequence (SS) for secretion and followed by a linker with an embedded KpnI restriction site. In step two, PCR is used to amplify the <2-microglobulin gene. Finally, in step three, restriction digestion at the KpnI site is used followed by ligation to combine the two fragments. The IgG1 heavy chain and <2-microglobulin proteins are separated by a 12 amino acid linker.

Step A. Production of VH Cassette/CH1/hinge

Standard PCR is used to amplify the full IgG1 from template available at Vaccinex. Specifically, Vaccinex has generated an IgG1 construct that allows for insertion of a variable gene of interest through BssHI and BstEII restriction sites. This construct is described elsewhere (U.S. Appl. Publ. No. 2002/0123057) and is available as template for PCR using the following primers: sense 5′ AATTGCGGCCGCAAACCATGGGATGGAGCTG TATCATC 3′ (SEQ ID NO:15) (NotI and NcoI sites in bold); and (8) anti-sense 5′ CGGGGTACCTGACCCACCGCCTCCTTTACCCGGAGACA GGGAGAG 3′ (SEQ ID NO:24) (linker is in bold; Kpnl site is bolded and underlined). The PCR product is gel purified according to standard procedure.

Step B

The DNA fragment encoding either mature <2-microglobulin, or <2-microglobulin and its 20-amino acid signal sequence, is generated as described in Example 4.

Step C. Assembled Chimeric Full IgG1-<2 Microglobulin Product

The above fragments “A” and “B” are joined by restriction digestion at the KpnI site followed by ligation employing standard protocols. The complete gene is designed for insertion into the expression vector pIRESbleo3 (Clontech). This strategy is not limited to the use of pIRESbleo3. Specifically, the use of other expression vector simply requires re-engineering of the restriction digestion sites flanking the complete construct (NotI and BamHI). Nucleotide and protein sequence is presented without a VH-gene. Any given VH-gene can be inserted between the BssHII (bold) and BstEII (dashed underline) sites.

The resulting nucleotide sequence encoding the chimeric Full IgG1-mature <2-microglobulin product is as follows:

(SEQ ID NO:33)

GCGGCCGCAAACCATGGGATGGAGCTGTATCATCCTCTTCTTGGTA
GCAACAGCTACAGGCGCGCATATGGTCACCGTCTCCTCAGCCTCCA
CCAAGGGCCCATCGGTCTTCCCCCTGGCACCCTCCTCCAAGAGCAC
CTCTGGGGGCACAGCGGCCCTGGGCTGCCTGGTCAAGGACTACTTC
CCCGAACCGGTGACGGTGTCGTGGAACTCAGGCGCCCTGACCAGC
GGCGTGCACACCTTCCCGGCTGTCCTACAGTCCTCAGGACTCTACT
CCCTCAGCAGCGTCGTGACCGTGCCCTCCAGCAGCTTGGGCACCCA
GACCTACATCTGCAACGTGAATCACAAGCCCAGCAACACCAAGGT
GGACAAGAAAGTTGAGCCCAAATCTTGTGACAAAACTCACACATG
CCCACCGTGCCCAGCACCTGAACTCCTGGGGGGACCGTCAGTCTTC
CTCTTCCCCCCAAAACCCAAGGACACCCTCATGATCTCCCGGACCC
CTGAGGTCACATGCGTGGTGGTGGACGTGAGCCACGAAGACCCTG
AGGTCAAGTTCAACTGGTACGTGGACGGCGTGGAGGTGCATAATG
CCAAGACAAAGCCGCGGGAGGAGCAGTACAACAGCACGTACCGTG
TGGTCAGCGTCCTCACCGTCCTGCACCAGGACTGGCTGAATGGCAA
GGAGTACAAGTGCAAGGTCTCCAACAAAGCCCTCCCAGCCCCCATC
GAGAAAACCATCTCCAAAGCCAAAGGGCAGCCCCGAGAACCACAG
GTGTACACCCTGCCCCCATCCCGGGATGAGCTGACCAAGAACCAG
GTCAGCCTGACCTGCCTGGTCAAAGGCTTCTATCCCAGCGACATCG
CCGTGGAGTGGGAGAGCAATGGGCAGCCGGAGAACAACTACAAGA
CCACGCCTCCCGTGCTGGACTCCGACGGCTCCTTCTTCCTCTACAGC
AAGCTCACCGTGGACAAGAGCAGGTGGCAGCAGGGGAACGTCTTC
TCATGCTCCGTGATGCATGAGGCTCTGCACAACCACTACACGCAGA
AGAGCCTCTCCCTGTCTCCGGGTAAAGGAGGCGGTGGGTCAGGT
ACCGGAGGCGGTGGGTCAATCCAGCGTACTCCAAGATTCAGGT
TTACTCACGTCATCCAGCAGAGAATGGAGTCAAATTTCCTGAAT
TGCTATGTGTCTGGGTTTCATCCATCCGACATTGAAGTTGACTTACT
GAAGAATGGAGAGAGAATTGAAAAAGTGGAGCATTCAGACTTGGT
GTTCAGCAAGGACTGGTCTTTCTATCTCTTGTACTACACTGAATTCA
CCCCCACTGAAAAAGATGAGTATGCCTGCCGTGTGAACCATGTGAC
TTTGTCACAGCCCAAGATAGTTAAGTGGGATCGAGACATGTAAGGA
TCCCG - 1419.

Double underline: NotI restriction site; single underline: signal sequence; bold: BssHII restriction site; double underline italics: BstEII restriction site; bold and underlined: linker; single underline italics: BamHI restriction site.

The polypeptide sequence of the chimeric full IgG1-mature <2-microglobulin product is:

(SEQ ID NO: 43)

MGWSCIILFLVATATGAHMVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVH
NAKTKPREEEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFFLYSKLTVDKSRWQQGNVFSCSV
MHEALHNHYTQKSLSLSPGKGGGGSGTGGGGSIQRTPKIQVYSRHPA
ENGKSNFLNCYVSGFHPSDIEVDLLKNGERIEKVEHSDLVFSKWSFY
LLYYTEFTPTEKDEYACRVNHVTLSQPKIVKWDRDM - 465.

The variable gene sequence is introduced at the histidine in bold and the rest of the bolded amino acids are removed upon insertion of the variable gene sequence. The linker is bolded and underlined.

The resulting nucleotide sequence encoding the chimeric full IgG1-<2-microglobulin product which retains the <2-microglobulin signal sequence is as follows:

(SEQ ID NO: 25)

GCGGCCGCAAACCATGGGATGGAGCTGTATCATCCTCTTCTTGGTA
GCAACAGCTACAGGCGCGCATATGGTCACCGTCTCCTCAGCCTCCA
CCAAGGGCCCATCGGTCTTCCCCCTGGCACCCTCCTCCAAGAGCAC
CTCTGGGGGCACAGCGGCCCTGGGCTGCCTGGTCAAGGACTACTTC
CCCGAACCGGTGACGGTGTCGTGGAACTCAGGCGCCCTGACCAGC
GGCGTGCACACCTTCCCGGCTGTCCTACAGTCCTCAGGACTCTACT
CCCTCAGCAGCGTCGTGACCGTGCCCTCCAGCAGCTTGGGCACCCA
GACCTACATCTGCAACGTGAATCACAAGCCCAGCAACACCAAGGT
GGACAAGAAAGTTGAGCCCAAATCTTGTGACAAAACTCACACATG
CCCACCGTGCCCAGCACCTGAACTCCTGGGGGGACCGTCAGTCTTC
CTCTTCCCCCCAAAACCCAAGGACACCCTCATGATCTCCCGGACCC
CTGAGGTCACATGCGTGGTGGTGGACGTGAGCCACGAAGACCCTG
AGGTCAAGTTCAACTGGTACGTGGACGGCGTGGAGGTGCATAATG
CCAAGACAAAGCCGCGGGAGGAGCAGTACAACAGCACGTACCGTG
TGGTCAGCGTCCTCACCGTCCTGCACCAGGACTGGCTGAATGGCAA
GGAGTACAAGTGCAAGGTCTCCAACAAAGCCCTCCCAGCCCCCATC
GAGAAAACCATCTCCAAAGCCAAAGGGCAGCCCCGAGAACCACAG
GTGTACACCCTGCCCCCATCCCGGGATGAGCTGACCAAGAACCAG
GTCAGCCTGACCTGCCTGGTCAAAGGCTTCTATCCCAGCGACATCG
CCGTGGAGTGGGAGAGCAATGGGCAGCCGGAGAACAACTACAAGA
CCACGCCTCCCGTGCTGGACTCCGACGGCTCCTTCTTCCTCTACAGC
AAGCTCACCGTGGACAAGAGCAGGTGGCAGCAGGGGAACGTCTTC
TCATGCTCCGTGATGCATGAGGCTCTGCACAACCACTACACGCAGA
AGAGCCTCTCCCTGTCTCCGGGTAAAGGAGGCGGTGGGTCAGGT
ACCGGAGGCGGTGGGTCAATGTCTCGCTCCGTGGCCTTAGCTGTG
CTCGCGCTACTCTCTCTTTCTGGCCTGGAGGCTATCCAGCGTACTCC
AAAGATTCAGGTTTACTCACGTCATCCAGCAGAGAATGGAAAGTC
AAATTTCCTGAATTGCTATGTGTCTGGGTTTCATCCATCCGACATTG
AAGTTGACTTACTGAAGAATGGAGAGAGAATTGAAAAAGTGGAGC
ATTCAGACTTGGTGTTCAGCAAGGACTGGTCTTTCTATCTCTTGTAC
TACACTGAATTCACCCCCACTGAAAAAGATGAGTATGCCTGCCGTG
TGAACCATGTGACTTTGTCACAGCCCAAGATAGTTAAGTGGGATCG
AGACATGTAAGGATCCCG - 1479.

Double underline: NotI restriction site; single underline: signal sequence; bold: BssHII restriction site; double underline italics: BstEII restriction site; bold and underlined: linker; single underline italics: BamHI restriction site.

The polypeptide sequence of the chimeric full IgG1-<2-microglobulin product which retains the <2-microglobulin signal sequence is:

(SEQ ID NO: 26)

MGWSCIILFLVATATGAHMVTVSSASTKGPSVFPLAPSSKSTSGGTAA
LGCLVKDYFPEPVTVSWNSGALTSGVHTFPAVLQSSGLYSLSSVVTVP
SSSLGTQTYICNVNHKPSNTKVDKKVEPKSCDKTHTCPPCPAPELLGG
PSVFLFPPKPKDTLMISRTPEVTCVVVDVSHEDPEVKYNWYVDGVEVH
NAKTKPREEQYNSTYRVVSVLTVLHQDWLNGKEYKCKVSNKALPAPI
EKTISKAKGQPREPQVYTLPPSRDELTKNQVSLTCLVKGFYPSDIAVE
WESNGQPENNYKTTPPVLDSDGSFFLYSKLTVDKSRWQQGNVFSCSV
MHEALHNHYTQKSLSLSPGKGGGGSGTGGGGSMSRSVALAVLALLS
LSGLEAIQRTPKIQVYSRHPAENGKSNFLNCYVSGFHPSDIEVDLLKNG
ERIEKVEHSDLVFSKDWSFYLLYYTEFTPTEKDEYACRVNHVTLSQPK
IVKWDRDM - 485.

The variable gene sequence is introduced at the histidine in bold and the rest of the bolded amino acids are removed upon insertion of the variable gene sequence. The linker is bolded and underlined.

### Example 7

To assemble a complete antibody molecule or fragment thereof, the chimeric immunoglobulin heavy chain or fragment thereof must be associated with a natural or chimeric immunoglobulin light chain and the chimeric immunoglobulin light chain must be associated with a natural or chimeric immunoglobulin heavy chain or fragment thereof chain. This can be accomplished either by co-synthesis in the same eukaryotic cell or by in vitro assembly of the separate chains. Note that it is possible to form molecules in which both immunoglobulin heavy and light chains are fused to <2-microglobulin.

To assemble a complete MHC Class I molecule, the MHC Class I α heavy chain must be associated with the chimeric antibody or antibody fragment-<2-microglobulin. In a preferred embodiment, this is accomplished by in vitro assembly with an MHC Class I α heavy chain separately synthesized in either eukaryotic or bacterial cells, as previously described (Altman et al. (1993), or Garboczi et al. (1992)).

Proper assembly and stability of this complex can be further enhanced by incorporating an MHC Class I α heavy chain to which a peptide has been fused at the amino terminus of the α chain which peptide is able to bind to the groove formed by the MHC Class I α1 and α2 domains as previously described Mottez et al., J. Exp. Med. 181:493 (1995).

### Example 8

### Chimeric Kappa L Chain-<2 Microglobulin

Employing the same strategy described above for fusion products with immunoglobulin heavy chain or heavy chain fragments, it is possible to create a chimeric kappa L chain coupled in frame with <2-microglobulin. Assembly takes place in a three-step process. In step one, PCR is employed to create the CL region preceded by a signal sequence for secretion and followed by a linker with an embedded KpnI restriction site. The kappa light chain constant region (CK) can be PCR amplified from a previously described plasmid template with a pre-configured VL insertion site that allows for directional cloning of any immunoglobulin light chain variable region gene of interest at ApaLI and XhoI restriction sites (U.S. Appl. Publ. No. 2002/0123057). In step two, the <2-microglobulin gene preceded by the linker with a KpnI restriction site is amplified exactly as described above for heavy chain fusion products. Finally in step three the two fragments are joined by restriction digestion at the KpnI site followed by ligation employing standard protocols. The modifications of primer sequences required for amplification of the immunoglobulin light chain with either kappa or lambda light chain constant regions will be apparent to those skilled in the art.

### Example 9

### Chimeric <2-microglobulin-Immunoglobulin Kappa Light Chain

The intent is to create a chimeric protein in which <2-microglobulin is fused through a linker to an immunoglobulin light chain that can associate with an immunoglobulin heavy chain or fragment thereof to form an antigen binding antibody or fragment thereof. Assembly takes place in a three-step process. In step A, custom PCR primers are used to amplify the <2-microglobulin gene including the signal sequence. In step B, custom PCR primers are used to amplify the immunoglobulin kappa light chain constant region (CK) from a previously described plasmid template with a pre-configured VK insertion site that allows for directional cloning of any immunoglobulin light chain variable region gene of interest at ApaLI and XhoI restriction sites (U.S. Appl. Publ. No. 2002/0123057). Using the primers described, the <2-microglobulin and IgG1 proteins are separated by a 12 amino acid linker. The length of the linker provided between <2-microglobulin and the immunoglobulin chain can be readily modified by those skilled in the art.

Step A

The <2-microglobulin fragment is generated as described in Example 1.

Step B. Production of the VL Cloning Cassette and the Kappa Light Chain Constant Region

Standard PCR is used to amplify the immunoglobulin kappa light chain constant region (CK) from a previously described plasmid template with a pre-configured VK insertion site that allows for directional cloning of any immunoglobulin light chain variable region gene of interest at ApaLI and XhoI restriction sites (U.S. Appl. Publ. No. 2002/0123057). The 5′ end of fragment “B” is designed to allow for ligation with the 3′ end of fragment “A” at a KpnI restriction site. The 3′ end of fragment “B” is designed with a linker containing a BamHI site to allow for cloning into pIRESbleo3. The PCR product is 387 nucleotides in length. Fragment “D” is generated by PCR using plasmid DNA as template (Source: Open Biosystems Inc.; Cat.#:, OBS#:, Source ID:, IMAGE ID:) and the following primers: sense 5′-CGGGGTACCGGAGGCGGTGGGTCAGCTACAGGCGTGCACTTGAC-3′ (SEQ ID NO:27) (linker is in bold; KpnI restriction site is bolded and underlined); and anti-sense 5′-CGGGATCC CTAACACTCTCCCCTGTTGAAG-3′(SEQ ID NO: 48) (BamHI restriction site is in bold).

In another embodiment, constant or variable regions from lambda light chains may be incorporated in fragment “B”.

Step C. Assembly of Chimeric <2 Microglobulin-kappa Light Chain

Fragments A and B separately undergo PCR amplification and gel purification using standard conditions. Each fragment is digested with KpnI to generate overlapping sites for ligation. The fragments are then ligated at the KpnI site. The resulting product is then digested with ClaI and BamHI to create overlapping fragments for ligation into a mammalian expression construct. The complete gene is designed for insertion into the retroviral expression vector pIRESbleo3 (Clontech). However, this strategy is not limited to the use of pIRESbleo3. Specifically, the use of other expression vectors simply requires re-engineering of the restriction digestion sites flanking the complete construct (ClaI and BamHI). Any given v-gene can be inserted between the ApaLI (bold) and XhoI (dashed underline) sites.

The final sequence is: ATCGATATGTCTCGCTCCGTGG CCTTAGCTGTGCTCGCGCTACTCTCTCTTTCTGGCCTGGAGGCTATC CAGCGTACTCCAAAGATTCAGGTTTACTCACGTCATCCAGCAGAGA ATGGAAAGTCAAATTTCCTGAATTGCTATGTGTCTGGGTTTCATCCA TCCGACATTGAAGTTGACTTACTGAAGAATGGAGAGAGAATTGAA AAAGTGGAGCATTCAGACTTGTCTTTCAGCAAGGACTGGTCTTTCT ATCTCTTGTACTACACTGAATTCACCCCCACTGAAAAAGATGAGTA TGCCTGCCGTGTGAACCATGTGACTTTGTCACAGCCCAAGATAGTT AAGTGGGATCGAGACATGGGAGGCGGTGGGTCAGGTACCGGAGGC GGTGGGTCAGCTACAGGCGTGCACTTGACTCGAGATCAAACGAACT GTGGCTGCACCATCTGTCTTCATCTTCCCGCCATCTGATGAGCAGTT GAAATCTGGAACTGCCTCTGTTGTGTGCCTGCTGAATAACTTCTATC CCAGAGAGGCCAAAGTACAGTGGAAGGTGGATAACGCCCTCCAAT CGGGTAACTCCCAGGAGAGTGTCACAGAGCAGGACAGCAAGGACA GCACCTACAGCCTCAGCAGCACCCTGACGCTGAGCAAAGCAGACT ACGAGAAACACAAAGTCTACGCCTGCGAAGTCACCCATCAGGGCC TGAGCTCGCCCGTCACAAAGAGCTTCAACAGGGGAGAGTGTTAGG GATCCCG-762 (SEQ ID NO:49) (bolded sequence: ClaI restriction site; underlined sequence: <2M signal sequence; double underline italics: ApaLI restriction site; single underline italics: XhoI restriction site; Bolded and underlined: BamHI restriction site).

The resultant polypeptide is:

(SEQ ID NO: 50)

MSRSVALAVLALLSLSGLE
AIQRTPKIQVYSRHPAENGKSNFLNCYVSGFHPSDIEVDLLKNGERIEK
VEHSDLSFSKDWSFYLLYYTEFTPTEKDEYACRVNHVTLSQPKIVKW
DRDMGGGGSGTGGGGSATGVHLEIKRTVAAPSVFIFPPSDEQLKSGTA
SVVCLLNNFYPREAKVQWKVDNALQSGNSQESVTEQDSKDSTYSLSS
TLTLSKADYEKHKVYACEVTHQGLSSPVTKSFNRGEC - 247.

Variable genes of interest can be inserted between ApaLI and XhoI. Point of insertion relative to protein sequence is denoted above in bold (between glycine and valine).

### Example 10

### Assembling the Chimeric <2-microglobulin-antibody or Antibody Fragment

To assemble a complete antibody molecule or fragment thereof, the chimeric <2-microglobulin-kappa light chain must be associated with an immunoglobulin heavy chain or fragment thereof. This can be accomplished either by co-synthesis in the same eukaryotic cell or by in vitro assembly of the separate chains.

To assemble a complete MHC Class I molecule, the MHC Class I α heavy chain must be associated with the chimeric <2-microglobulin-antibody or antibody fragment. In a preferred embodiment, this is accomplished by in vitro assembly with an MHC Class I α heavy chain separately synthesized in either eukaryotic or bacterial cells, as previously described (see Altman et al. (1993), or Garboczi et al. (1992)). In a preferred embodiment, the proper assembly of this complex comprising a <2-microglobulin-antibody or antibody fragment chimeric molecule and MHC Class I α heavy chain is facilitated by introduction of a three nucleotide mutation at position 222-224 of the <2-microglobulin open reading frame which results in the substitution of a valine for serine at amino acid 74 of the <2-microglobulin protein. For this purpose, the plasmid DNA employed for production of fragment “A” above is modified to incorporate this mutation by methods well known to those skilled in the art.

Proper assembly and stability of this complex can be further enhanced by incorporating an MHC Class I α heavy chain to which a peptide has been fused at the amino terminus of the α chain which peptide is able to bind to the groove formed by the MHC Class I α1 and α2 domains as previously described (Mottez et al., J. Exp. Med. 181:493 (1995)).

### Example 11

### Chimeric Antigenic Peptide-<2-microglobulin-F(ab) Fragment

The intent is to create a chimeric protein in which an MHC Class I restricted peptide is fused through a linker to <2-microglobulin which is in turn fused through a second linker to VH and CH1 that can associate with an immunoglobulin light chain to form an antigen binding F(ab) fragment of a human IgG antibody. The method of assembling the construct is illustrated for an immunodominant peptide of Human Cytomegalovirus (CMV). Importantly, any epitope can be substituted through the creation of custom oligonucleotides in steps A and B. In addition, the length of the linker provided between the antigenic peptide and <2-microglobulin or between <2-microglobulin and the immunoglobulin chain can be readily modified by those skilled in the art. Finally, BssHI and BstEII sites are provided that will allow any immunoglobulin heavy chain variable region (VH) required for binding to a specific antigen to be inserted in frame into the antibody F(ab) fragment.

Step A. Assembling the <2-microglobulin Signal Sequence (“A” fragment)

Custom synthesized oligonucleotides are employed corresponding to the following sequences: sense 5′-CCATCGATATGTCTCGCTCCGTGGCCTTAGCTGTGCTCGCGCTACTCTCTCTTTC TGGCCTGGAGGCTAACCTGGTGCCCATG-3′ (SEQ ID NO:51) (ClaI restriction site is in bold; <2-microglobulin signal sequence is underlined once; nucleotides 1-15 of the CMV epitope is underlined twice); and anti-sense 5′-CATGGGCACCAGGTTAGCCTCCAGGCCAGAAAGA GAGAGTAGCGCGAGCACAGCTAAGGCCACGGAGCGAGACATATCG ATGG-3′ (SEQ ID NO: 52).

Double stranded <2-microglobulin signal sequence is generated by resuspending the custom sense and antisense oligonucleotides at the same concentration (100 mM) and then mixing them in equi-molar ratios. The mixture is then heated to 95<C for 2 minutes and allowed to gradually cool to 25<C over a period of 45 minutes. The double stranded complex is then gel purified on a 2% agarose gel. The 5′ end contains a ClaI endonuclease restriction site designed to allow for insertion into an expression construct. The 3′ end of the oligonucleotide is designed to allow for overlap extension PCR with the fragment described in section B.

Step B. Creating a “B” Fragment Comprised of the Entire CMV Epitope, a 15 Amino Acid Linker, and Nucleotides 1-15 of the Body of the <2-microglobulin Gene

Custom synthesized oligonucleotides are employed corresponding to the following sequences:sense 5′-AACCTGGTGCCCATGGTGGCTACG GTTGGAGGTGGGGGAGGCGGATCAGGAGGCTCAGGTGGGTCAGGAGGCATCCAGCGTACTCCA-3′ (SEQ ID NO:53) (the complete CMV epitope coding sequence is in bold; the 15 amino acid linker is underlined once; the first 15 nucleotides of the body of the <2-microglobulin gene is underlined twice); and anti-sense 5′-TGGAGTACGCTGGATGCCTCCTGACCCACCTGAGCCTCCTGATCCG CCTCCCCCACCTCCAACCGTAGCCACCATGGGCACCAGGTT-3′ (SEQ ID NO:34).

Double stranded “B” fragment is generated by resuspending the custom oligonucleotides at the same concentration (100 mM) and then mixing them in equi-molar ratios. The mixture is then heated to 95<C for 2 minutes and allowed to gradually cool to 25<C over a period of 45 minutes. The double stranded complex is then gel purified on a 2% agarose gel. The 5′ end is designed to allow for overlap extension PCR with the fragment described in section A above. The 3′ end is designed to allow for overlap extension PCR with the fragment described in section C below.

Step C. Creating the “C” Fragment Containing the Body (Minus the Signal Sequence) of the Human <2-microglobulin Gene

The 5′ end of fragment “C” is designed to allow for overlap extension PCR with the 3′ end of fragment “B” above. The 3′ end is designed to allow for ligation with the 5′ end of fragment “D” below. The PCR product is 321 nucleotides in length. The fragment is generated by standard PCR using plasmid DNA as template (Source: Open Biosystems Inc.; Cat.#: EHS1001, OBS#: 26266, Source ID: 5502428, IMAGE ID: 5502428) and the following primers: (5) Sense 5′-ATCCAGCGTACTCCAAAGATT-3′ (SEQ ID NO:35); and (6) Anti-sense 5′-CGGGGTACCTGACCCACCGCCTCCCATGTCTCGATCCCACTTAAC-3′ (SEQ ID NO:36) (linker is in bold; KpnI site is bolded and underlined). The PCR product is gel purified according to standard procedures.

Step D. Creating the “D” Fragment Containing the Cloning Site for VH and the Coding Sequence for CH1 of Human IgG.

Standard PCR is used to amplify CH1 from a previously described plasmid template with a pre-configured VH insertion site that allows for directional cloning of any immunoglobulin heavy chain variable region gene of interest at BssHI and BstEII restriction sites (U.S. Appl. Publ. No. 2002/0123057). The 5′ end of fragment “D” is designed for ligation with the 3′ end of fragment “C” at a KpnI restriction site. The 3′ end of fragment “D” is designed with a linker containing a BamHI site to allow for cloning into pIRESbleo3. The PCR product is 354 nucleotides in length. Fragment “D” is generated by PCR using plasmid DNA as template (Source: Open Biosystems Inc.; Cat.#: MHS1011, OBS#: 61678, Source ID: 4308411, IMAGE ID: 4308411) and the following primers: (7) sense 5′-CGGGGTACCGGAGGCGGTGGGTCAGGCGCGCATATGGTCACC-3′ (SEQ ID NO:37) (linker is in bold.; KpnI restriction site is bolded and underlined.); and (8) anti-sense 5′-CGGGGATCCCTATTTCTTGTCCACCTTGGTGTT-3′ (SEQ ID NO:38) (BamHI site is bolded; stop codon is underlined). In other embodiments, the CH1 region derives from other immunoglobulin isotypes, including IgG2, IgG3, IgG4, IgA, IgM, IgD or IgE.

Step E. Assembling the CMV-<2-microglobulin-CH1 Chimera

Step 1. The double stranded oligonucleotides from steps A and B are assembled in an overlap extension PCR assay according to standard protocols. The resulting product is 155 nucleotides in length. This product is gel purified according to standard protocols.

Step 2. Fragments C and D are independently created via PCR and gel purified according to standard protocols. Each fragment is then digested separately with KpnI to create overhangs used in the ligation reaction in step 3.

Step 3. Fragments C and D from step 2 are ligated according to standard protocols. The resulting product is 663 nucleotides. This product is gel purified.

Step 4. The purified 155 nucleotide product from step 1 and the 663 nucleotide product from step 3 are combined in an overlap extension PCR reaction. The resulting product is 803 nucleotides in length and is gel purified according to standard protocols.

Step 5. The purified product from step 4 can then be digested with ClaI and BamHI and inserted in the expression construct of interest.

The complete sequence of the chimeric construct is: CCATCGATATGTCTCGCTCCGTGGCCTTAGCTGTGCTCGCGCTACT CTCTCTTTCTGGCCTGGAGGCTAACCTGGTGCCCATGGTGGCTACG GTTGGAGGTGGGGGAGGCGGATCAGGAGGCTCAGGTGGGTCAGGA GGCATCCAGCGTACTCCAAAGATTCAGGTTTACTCACGTCATCCAG CAGAGAATGGAAAGTCAAATTTCCTGAATTGCTATGTGTCTGGGTT TCATCCATCCGACATTGAAGTTGACTTACTGAAGAATGGAGAGAGA ATTGAAAAAGTGGAGCATTCAGACTTGTCTTTCAGCAAGGACTGGT CTTTCTATCTCTTGTACTACACTGAATTCACCCCCACTGAAAAAGAT GAGTATGCCTGCCGTGTGAACCATGTGACTTTGTCACAGCCCAAGA TAGTTAAGTGGGATCGAGACATGGGAGGCGGTGGGTCAGGTACCG GAGGCGGTGGGTCAGGCGCGCATATGGTCACCGTCTCCTCAGCCTC CACCAAGGGCCCATCGGTCTTCCCCCTGGCACCCTCCTCCAAGAGC ACCTCTGGGGGCACAGCGGCCCTGGGCTGCCTGGTCAAGGACTACT TCCCCGAACCGGTGACGGTGTCGTGGAACTCAGGCGCCCTGACCAG CGGCGTGCACACCTTCCCGGCTGTCCTACAGTCCTCAGGACTCTAC TCCCTCAGCAGCGTCGTGACCGTGCCCTCCAGCAGCTTGGGCACCC AGACCTACATCTGCAACGTGAATCACAAGCCCAGCAACACCAAGG TGGACAAGAAATAGGGATCCCCG-803 (SEQ ID NO:39) (bolded sequence: ClaI restriction site; underlined sequence: <2M signal sequence; double underline: CMV peptide; double underline italics: BssHI restriction site; single underline italics: BstEII restriction site; bolded and underlined: BamHI restriction site).

The resultant polypeptide is:

(SEQ ID NO: 40)

MSRSVALAVLALLSLSGLEANLVPM
VATVGGGGGGSGGSGGSGGIQRTPKIQVYSRHPAENGKSNFLNCYVS
GFHPSDIEVDLLKNGERIEKVEHSDLSFSKDWSFYLLYYTEFTPTEKDE
YACRVNHVTLSQPKIVKWDRDMGGGGSGTGGGGSGAHMVTVSSAS
TKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKK -
261.

Variable genes of interest can be inserted between BssHI and BstEII. Point of insertion relative to protein sequence is denoted above in bold (between histidine and methionine).

The above strategy for generation of an antigenic peptide-<2-microglobulin fusion to the F(ab) fragment of human IgG1, can readily be adapted to fusion of antigenic peptide-<2-microglobulin to an F(ab′)2 antibody fragment including an immunoglobulin hinge region or to a complete IgG immunoglobulin heavy chain. Specifically, one would simply have to reengineer primer (8) in this document to create a D fragment that contains not only CH1 but the hinge region (for F(ab′)2) or the hinge region, CH2, and CH3 for the complete IgG heavy chain. The following primers would be sufficient replacements for primer (8) to accomplish this task.

For F(ab′)2 fusion: (9) Anti-sense 5′-CGGGGATCC CTATGGGCACGGTGGGCATGTGTG-3′ (SEQ ID NO:41) (BamHI site is bolded; stop codon is underlined).

For full IgG immunoglobulin heavy chain fusion: (10) Anti-sense 5′-CGGGGATCCCTATTTACCCGGAGACAGGGAGAG-3′ (SEQ ID NO:42) (BamHI site is bolded; stop codon is underlined).

Step F. Assembling the Chimeric CMV-<2-microglobulin-antibody or Antibody Fragment

To assemble a complete antibody molecule or fragment thereof, the chimeric antigenic peptide-<2-microglobulin-immunoglobulin heavy chain or fragment thereof must be associated with an immunoglobulin light chain. This can be accomplished either by co-synthesis in the same eukaryotic cell or by in vitro assembly of the separate chains.

Step G. Assembling a Complete MHC Class I Molecule on the Chimeric CMV-<2-microglobulin-antibody or Antibody Fragment

To assemble a complete MHC Class I molecule, the MHC Class I α heavy chain must be associated with the chimeric peptide-<2-microglobulin-antibody or antibody fragment. In a preferred embodiment, this is accomplished by in vitro assembly with an MHC Class I α heavy chain separately synthesized in either eukaryotic or bacterial cells, as previously described (see Altman et al. (1993), or Garboczi et al. (1992))). The proper assembly of this complex comprising a chimeric peptide-<2-microglobulin-antibody or antibody fragment and MHC Class I α heavy chain is facilitated by the added affinity of the selected peptide for the peptide binding site of the MHC Class I α heavy chain.

### Example 12

### Chimeric Antigenic Peptide-<2-microglobulin Immunoglobulin Kappa Light Chain

The intent is to create a chimeric protein in which an MHC Class I restricted peptide is fused through a linker to <2-microglobulin which is in turn fused through a second linker to an immunoglobulin light chain that can associate with an immunoglobulin heavy chain or fragment thereof to form an antigen binding antibody or fragment thereof. The method of assembling the construct is illustrated for an immunodominant peptide of Human Cytomegalovirus (CMV). Importantly, any epitope can be substituted through the creation of custom oligonucleotides in steps A and B. In addition, the length of the linker provided between the antigenic peptide and <2-microglobulin or between <2-microglobulin and the immunoglobulin chain can be readily modified by those skilled in the art. Finally, BssHI and BstEII sites are provided that will allow any immunoglobulin light chain variable region (VL) required for binding to a specific antigen to be inserted in frame into the light chain fragment.

The “A”, “B” and “C” fragments are generated as described above in Example 11.

Step D. Creating the “D” Fragment Containing the Cloning Site for V_{L} and the Kappa Light Chain Constant Region

Standard PCR is used to amplify the immunoglobulin kappa light chain constant region (CK) from a previously described plasmid template with a pre-configured VK insertion site that allows for directional cloning of any immunoglobulin light chain variable region gene of interest at ApaLI and XhoI restriction sites (U.S. Appl. Publ. No. 2002/0123057). The 5′ end of fragment “D” is designed to allow for ligation with the 3′ end of fragment “C” at a KpnI restriction site. The 3′ end of fragment “D” is designed with a linker containing a BamHI site to allow for cloning into pIRESbleo3. The PCR product is 387 nucleotides in length. Fragment “D” is generated by PCR using plasmid DNA as template (Source: Open Biosystems Inc.; Cat.#:, OBS#:, Source ID:, IMAGE ID:) and the following primers: (7) sense 5′-CGGGGTACCGGAGGCGGTGGGTCAGCTACAGGCGTGCACTTGAC-3′ (SEQ ID NO:54); (linker is in bold; and KpnI restriction site is bolded and underlined); and (8) anti-sense 5′-CGGGATCCCTAACACTCTCCCCTGTTGAAG-3′ (SEQ ID NO:44) (BamHI restriction site is in bold).

In another embodiment, constant or variable regions from lambda light chains may be incorporated in fragment “B”.

Step E. Assembling the CMV-<2-microglobulin-kappa Light Chain Chimera

Step 1. The double stranded oligonucleotides from steps A and B are assembled in an overlap extension PCR assay according to standard protocol. The resulting product is 155 nucleotides in length. This product is gel purified according to standard protocol.

Step 2. Fragments C and D are independently created via PCR and gel purified according to standard protocol. Each fragment is then digested separately with KpnI to create overhangs used in the ligation reaction in step 3.

Step 3. Fragments C and D from step 2 are ligated according to standard protocol. The resulting product is 696 nucleotides. This product is gel purified.

Step 4. The purified 155 nucleotide product from step 1 and the 696 nucleotide product from step 3 are combined in an overlap extension PCR reaction. The resulting product is 836 nucleotides in length and is gel purified according to standard protocol.

Step 5. The purified product from step 4 can then be digested with ClaI and BamHI and inserted in the expression construct of interest.

The complete sequence of the chimeric construct is: CCATCGATATGTCTCGCTCCGTGGCCTTAGCTGTGCTCGCGCTACT CTCTCTTTCTGGCCTGGAGGCTAACCTGGTGCCCATGGTGGCTACG GTTGGAGGTGGGGGAGGCGGATCAGGAGGCTCAGGTGGGTCAGGA GGCATCCAGCGTACTCCAAAGATTCAGGTTTACTCACGTCATCCAG CAGAGAATGGAAAGTCAAATTTCCTGAATTGCTATGTGTCTGGGTT TCATCCATCCGACATTGAAGTTGACTTACTGAAGAATGGAGAGAGA ATTGAAAAAGTGGAGCATTCAGACTTGTCTTTCAGCAAGGACTGGT CTTTCTATCTCTTGTACTACACTGAATTCACCCCCACTGAAAAAGAT GAGTATGCCTGCCGTGTGAACCATGTGACTTTGTCACAGCCCAAGA TAGTTAAGTGGGATCGAGACATGGGAGGCGGTGGGTCAGGTACCG GAGGCGGTGGGTCAGCTACAGGCGTGCACTTGACTCGAGATCAAAC GAACTGTGGCTGCACCATCTGTCTTCATCTTCCCGCCATCTGATGAG CAGTTGAAATCTGGAACTGCCTCTGTTGTGTGCCTGCTGAATAACT TCTATCCCAGAGAGGCCAAAGTACAGTGGAAGGTGGATAACGCCC TCCAATCGGGTAACTCCCAGGAGAGTGTCACAGAGCAGGACAGCA AGGACAGCACCTACAGCCTCAGCAGCACCCTGACGCTGAGCAAAG CAGACTACGAGAAACACAAAGTCTACGCCTGCGAAGTCACCCATC AGGGCCTGAGCTCGCCCGTCACAAAGAGCTTCAACAGGGGAGAGT GTTAGGGATCCCG-836 (SEQ ID NO:45) (bolded sequence: ClaI restriction site; underlined sequence: <2M signal sequence; double underline: CMV peptide; double underline italics: ApaLI restriction site; single underline italics: XhoI restriction site; bolded and underlined: BamHI restriction site).

The resultant polypeptide is:

(SEQ ID NO: 46)

MSRSVALAVLALLSLSGLEANLVP
MVATVGGGGGGSGGSGGSGGIQRTPKIQVYSRHPAENGKSNFLNCYV
SGFHPSDIEVDLLKNGERIEKVEHSDLSFSKDWSFYLLYYTEFTPTEKD
EYACRVNHVTLSQPKIVKWDRDMGGGGSGTGGGGSATGVHLEIKRT
VAAPSVFIFPPSDEQLKSGTASVVCLLNNFYPREAKVQWKVDNALQS
GNSQESVTEQDSKDSTYSLSSTLTLSKADYEKHKVYACEVTHQGLSSP
VTKSFNRGEC - 271.

Variable genes of interest can be inserted between ApaLI and XhoI. Point of insertion relative to protein sequence is denoted above in bold (between glycine and valine).

Steps F and G

The complete antibody molecule or fragment thereof, and the complete MHC clss I molecule is assembled as described in Example 11, above.

### Example 13

### Assay for the in vitro Activity of Compounds of the Invention Targeted to Dendritic Cells.

Dendritic cells are the most potent stimulators of T cell responses identified to date. To test in vitro activity of compounds of the invention specifically targeted to dendritic cells, DC are incubated with the relevant compounds and assayed for the ability to activate human autologous T lymphocytes. Immature dendritic cells are prepared from healthy donors according to the method of Bhardwaj and colleagues (Reddy, A. et al., Blood 90:3640-3646 (1997)). Briefly, PBMC are incubated with neuraminidase-treated sheep erythrocytes and separated into rosetted T cell (ER+) and non-T cell (ER−) fractions. The ER+ fraction is cryopreserved for later use. The ER− fraction (2×10⁶ cells per well) is cultured in serum-free RPMI medium containing 1000 U/ml rhGM-CSF, 1000 U/ml rhIL-4 and 1% autologous plasma. This medium is replenished every other day. At day 7, the non-adherent immature DC are harvested from the culture and re-plated in maturation conditions (1000 U/ml GM-CSF, 1000 U/ml IL-4, 1% autologous plasma and 12.5-50% monocyte-conditioned medium) for 2-4 days. Cells manipulated in this manner have morphological and surface characteristics (CD83⁺) of mature DC.

Mature (or immature) DC are pulsed with compounds of the invention, or with free peptide or free MHC/peptide tetramers as controls for a short period followed by cocultivation with autologous T cells in 24 well plates for a period of 7-14 days. In some experiments, these may be total T lymphocytes, but it may also be desirable to fractionate CD4 and CD8 cells using magnetic separation systems (Miltenyi Biotech). Total T lymphocytes are incubated with the appropriate antibody-magnetic bead conjugates to isolate total CD4, CD8, naïve CD4+CD45RA+, naïve CD8+CD45RA+, memory CD4+CD45RO+ or memory CD8+CD45RO+ lymphocytes. For naïve CD4 and CD8 lymphocytes, a cytokine cocktail consisting of IL-2 (20 U/ml), IL-12 (20 U/ml), IL-18 (10 ng/ml), IFN-gamma (1 ng/ml) and a monoclonal antibody specific for IL-4 (50 ug/ml) is especially potent in enhancing DC activation of cytotoxic T cells in vitro. Following the activation period, CTL activity is assessed in a 4 hour ⁵¹Cr release assay. Other in vitro assays of T cell activation include proliferation (measured by increases in ³H-Thymidine incorporation or colorimetric MTT assay), cytokine secretion (IFN-γ, TNF-α, GM-CSF, IL-2) measured by ELISA, ELISpot, or flow cytometric detection (Luminex bead system). Many of these methods are described in Current Protocols in Immunology (John Wiley & Sons, New York). These and other methods are well known to those practiced in the art. Enhancement of T cell responses to targeted compounds of the invention is determined by comparison to the response to equimolar concentrations of free peptide or untargeted peptide-MHC Class I tetramers.

### Example 14

### Assay for T Cell Proliferation

T cell proliferation can be determined in vitro in a standard assay of ³H-Thymidine uptake and cytotoxic activity can be assayed by ⁵¹Cr release from labeled targets. For example, T cells are treated in vitro with monovalent antibody specific for CD28 costimulator molecules linked to monomeric or polymeric complexes of the influenza matrix peptide (58-66) bound to HLA-A2. Following in vitro culture for 6 days, influenza specific cytotoxic activity is assessed in a standard 4 hour ⁵¹Cr release assay with ⁵¹Cr labeled targets that have been pulsed with either heat killed influenza virus or the specific influenza matrix peptide employed in the stimulating peptide-MHC Class I complexes. The simultaneous delivery to a specific T cell of both ligand for the specific T cell receptor and costimulatory signal via the linked anti-CD28 antibody is expected to greatly enhance that T cell response. Enhancement of T cell responses to compounds of the invention is determined by comparison to the response to equimolar concentrations of the same free peptide or untargeted peptide-MHC Class I complexes.

### Example 15

### Assay for in vivo T Cell Expansion Following Stimulation with Compounds of the Invention

The effect of targeted vaccine complexes on expansion of specific T cells in vivo in either humans or HLA transgenic mice is determined by recovering T cells before and at intervals following immunization with a specific vaccine complex and determining the frequency of T cells specific for the vaccine complex by staining with tetrameric complexes of the same peptide-MHC Class I. Tetramers comprising the same peptide MHC complex of interest are employed in a cell surface immunofluorescence assay as follows. HLA-transgenic mouse spleen, lymph node or peripheral blood cells (collected by tail or retro-orbital bleeding) or human PBMC (1-10⁵ cells per sample) are incubated on ice in the presence of azide with control or experimental tetramers for about 30 minutes. After washing 2-3 times with staining buffer (such as PBS 1% BSA, 0.1% azide) a secondary streptavidin-fluorochrome (FITC, PE, or other fluorochrome) conjugate is added. After incubating for about 30 minutes, the samples are again washed 2-3 times and immunofluorescence is detected using a flow cytometer. These data are compared to pre-vaccination flow cytometric profiles to determine percentage increase in T cell precursor frequency and are repeated multiple times during the course of an experiment or clinical trial.

### Example 16

### In vitro Assays for Tumoricidal Activity of T Cells Specifically Targeted to Tumors by Compounds of the Invention

To demonstrate the ability to redirect cytotoxic T cells to the desired tumor target, tumor cells are incubated with compounds of the invention comprised of a tumor-specific antibody linked to peptide-MHC Class I complexes for which T cells are prevalent (eg HLA-A*0201 associated with influenza matrix peptide 58-66 ). ⁵¹Cr (100 μCi) is added during this 1 hour incubation to label the tumor cells. Following 2-3 washes, influenza specific CTL restricted to the appropriate MHC molecule (in this case, HLA-A2) are added at various effector to target (E:T) ratios in a 4 hr chromium release assay. Increased tumor lysis in the experimental sample containing compounds of the invention relative to control compounds with irrelevant peptide-MHC Class I complexes or tumor-specific antibody unlinked to peptide-MHC Class I complexes demonstrates that the compound of interest successfully sensitizes tumors to lysis by CTL specific for influenza virus.

The previous paragraph demonstrates redirection of cytotoxic effector function of influenza peptide-specific CTL to uninfected tumor cells by compounds of the invention that comprise a tumor specific antibody and influenza peptide-MHC Class I complexes. To demonstrate the ability of tumor cells treated with the same compound to induce an influenza peptide-specific T cell response, total T cells or CD8⁺CD45RA⁺ naïve T cells (1-2×10⁶ per well ) are stimulated in 24 well plates with tumor cells (1×10⁵) pulsed with compounds of the invention linked to MHC tetramers with influenza matrix peptide. Cytokines such as IL-2, IL-12, IL-18, IFN-γ may also be added to enhance activation of naïve CTL. Induction of cytotoxic T lymphocytes is assessed in a standard ⁵¹Cr release assay, described below.

This same method of targeting peptide-MHC Class I complexes to the tumor cell surface can be employed to enhance MHC-restricted presentation of known tumor-specific peptides; and, more, generally, to overcome immune evasion by tumor cells through downregulation of MHC molecules on the tumor surface. Compounds of the invention that comprise one or more tumor-specific antibodies linked to peptide-MHC Class I complexes would sensitize even tumor targets that have downregulated endogenous MHC to lysis by CTL specific for that same peptide-MHC Class I complex.

### Example 17

### In vivo Assays for Tumoricidal Activity of T Cells Specifically Targeted to Tumors by Compounds of the Invention

In a murine experimental model, compounds of the invention can be targeted to tumor cells through a naturally occurring or transfected tumor membrane marker. For example, BALB/c tumors such as EMT-6 (mammary carcinoma, Rockwell, S C et al., J. Natl. Cancer Inst. 49:735-749 (1972)), Line 1 (small cell lung carcinoma, Yuhas, J. M. et al., Cancer Res. 34:722-728 (1974)) or BCA (fibrosarcoma, Sahasrabudhe, D. M. et al., J. Immunology 151: 6302-6310 (1993)) may be transfected with a model antigen (e.g. chicken egg ovalbumin, OVA) for which antibodies are commercially available or easily made by the skilled artisan. More preferably, a BALB/c mammary tumor such as EMT-6 or SM1 (Hurwitz, A. A. et al., Proc. Nat. Acad. Sci. USA 95:10067-71 (1998)) is employed that expresses the murine homolog of the human C35 protein previously shown to be differentially expressed on the surface of human mammary tumor cells. Antibodies or antibody fragments specific for this model antigen may be linked to peptide-MHC Class I complexes that are either naturally occurring in that tumor, such as the L3 ribosomal protein peptide 48-56 expressed in association with H-2K^{d} in the BCA tumors, or a well-characterized pathogenic peptide known to induce a high frequency of high avidity T cells, such as the peptide-MHC Class I complex comprised of the HIV gp 160IIIB peptide RGPGRAFVTI (SEQ ID NO:55) in association with H-2D^{d} (Shirai, M. et al., J. Immunol. 148:1657 (1992)).

BALB/c (H-2^{d}) mice with established mammary tumors and/or distant metastases expressing the targeted molecule (e.g. C35) and that have been immunized with a vaccinia recombinant of HIV gp160IIIB are injected with gp160IIIB peptide complexes of H-2D^{d} linked to an anti-C35 antibody specificity for targeting to tumor cells. The effect on tumor growth of treatment with these compounds of the invention is monitored by caliper measurements every other day.

This analysis can be extended to human tumors implanted in immunodeficient (SCID, Rag-1^{-/-}, or Rag-1^{-/-} common γ chain double knockout) mice. Following establishment of tumors in vivo, mice receive an injection(s) of compounds of the invention specific for human tumor antigens conjugated to MHC tetramers bearing the HLA-A2 restricted influenza peptide (or a control peptide). Influenza specific human CTL are adoptively transferred and tumor regression is monitored.

In clinical trials, a standard influenza vaccination may be added to the protocol to increase influenza specific CTL directed at the tumor by compounds of the invention comprising influenza peptide-MHC Class I complexes.

### Example 18

### Inhibition of EAE Induction in SJL Mice

Experimental allergic encephalomyelitis (EAE) is an autoimmune disease in mice and serves as an animal model for multiple sclerosis. Encephalitogenic regions of two proteins, myelin basic protein (MBP 91-103) and proteolipoprotein (PLP 139-151), have been defined. In the susceptible SJL mouse strain, EAE can be induced to develop following immunization with the encephalitogenic peptide or adoptive transfer of MBP-reactive T cells. To determine whether treatment with a compound of the invention (such a compound comprising MBP 91-103 or PLP139-151 as the antigenic peptide) will prevent EAE development after T cell activation, SJL mice can be injected with the compound of interest.

To induce EAE in SJL mice with MBP 91-103, mice are immunized with 400 μg of MBP 91-103 in complete Freund's adjuvant on the dorsum. Ten to 14 days later, regional draining lymph node cells are harvested and cultured in 24-well plates at a concentration of 6×10⁶ cells per well in 1.5 ml of RPMI 1640 medium/10% fetal bovine serum/1% penicillin/streptomycin with the addition of MBP at 50 μg/ml. After a 4-day in vitro stimulation, MBP 91-103-reactive T cell blasts are harvested via Ficoll/Hypaque density gradient, washed twice in PBS, and 1.3×10⁷ cells are injected into each mouse. Mice receiving encephalitogenic MBP 91-10³-reactive T cells then receive either 100 μg of a compound of the invention or normal saline on days 0, 3, and 7 i.v. (total dose 300 μg). Clinical and histological evaluations are performed to determine whether the compound of interest inhibited the development of EAE in these mice.

Alternatively, to induce EAE in SJL mice with PLP peptide 139-151, mice are immunized with PLP peptide 139-151 dissolved in PBS and mixed with complete Freund's adjuvant containing Mycobacterium tuberculosis H37Ra at 4 mg/ml in 1:1 ratio. Mice are injected with 150 μg of peptide adjuvant mixture. On the same day and 48 hours later, all animals are given 400 ng of pertussis toxin. Adoptive transfer of EAE are then performed as described above. Clinical and histological evaluations are performed to determine whether the compound of interest inhibited the development of EAE in these mice.

### Example 19

### T Cell Stimulation in Mice Treated with Compounds of the Invention

The effects of compounds of the invention on clonal expansion of peptide-specific T cell lines in vivo can be suitably examined in accordance with the following assay.

5 BALB/c mice are injected intraperitoneally with 10-100 μg of a compound of interest in PBS and 24 hours later injected subcutaneously at the base of the tail with 50 μg of peptide-KLH conjugate. The peptide in the antigenic peptide-KLH conjugate is the same antigenic peptide in the compound of interest. 5 BALB/c mice are injected with peptide-KLH conjugate alone. 5 BALB/c mice are injected with PBS. These injections are repeated 6 and 7 days later. Seven days after completion of the second set of injections, the mice are sacrificed. The inguinal and paraaortic lymph nodes are removed and rendered into a single cell suspension.

The suspension is depleted of antigen presenting cells by incubation on nylon wool and Sephadex G-10 columns, and the resulting purified T cell populations incubated with APCs pulsed with the peptide. Activated B cells from BALB/c mice are used at antigen presenting cells in the proliferation assay. B cells are prepared by culturing spleen cells with 50 μg/ml of LPS for 48 to 72 hours at which time activated cells are isolated by density gradient centrifugation on Lymphoprep. Activated B cells are then pulsed with the peptide for 3 hours, washed extensively, fixed with paraformaldehyde to inhibit proliferation of B cells, and added to purified T cells from each panel of mice.

The proliferation assay is carried out in 96 well round bottom microtiter plates at 37° C., 5% CO₂ for 3-5 days. Wells are pulsed with 1 μCi of ³H-thymidine for 18 hours prior to termination of cultures and harvested using a Skatron cell harvester. Incorporation of ³H-thymidine into DNA as a measure of T cell proliferation is determined using an LKB liquid scintillation spectrometer. The degree of peptide-reactive T cell proliferation is indicative of the T cell responses (i.e. of clonal expansion) that took place in the mice following immunization.

### Example 20

### Detection of Peptide Specific T Cells Following Induction of Immune Response

In order to determine whether injection of a compound of the invention has successfully immunized mice to mount a T cell response to ovalbumin, an ovalbumin specific T cell proliferation assay can be employed. Mice are immunized by the protocol described in Example 19 and T cells are prepared from the inguinal and paraaortic lymph nodes 6 days after the second immunization.

The suspension is depleted of antigen presenting cells by incubation on nylon wool and Sephadex G-10 columns, and the resulting purified T cell populations incubated with APCs pulsed with the antigenic peptide. Activated B cells from BALB/c mice are used as antigen presenting cells in the proliferation assay. B cells are prepared by culturing spleen cells with 50 μg/ml of LPS for 48 to 72 hours at which time activated cells are isolated by density gradient centrifugation on Lymphoprep. Activated B cells are then pulsed with the antigenic peptide for 3 hours, washed extensively, fixed with paraformaldehyde to inhibit proliferation of B cells, and added to purified T cells.

The proliferation assay is carried out in 96 well round bottom microtiter plates at 37° C., 5% CO₂ for 3-5 days. Wells are pulsed with 1 μCi of ³H-thymidine for 18 hours prior to termination of cultures and harvested using a Skatom cell harvester. Incorporation of ³H-thymidine into DNA as a measure of T cell proliferation is determined using an LKB liquid scintillation spectrometer. The degree of peptide-reactive T cell proliferation is indicative of the T cell responses (i.e. of clonal expansion) that took place in the mice following immunization.

### Example 21

### Antibody Dependent Targeting of Exogenous Peptide-MHC Class I Complexes to Cell Surface Membranes is Sufficient to Stimulate Specific T Lymphocytes

Biotinylated anti-CD19 antibody (1 μl of 0.7 μg/ml) is added to 5×10⁵ EBV-B cells in a total volume of 0.1 ml. CD19 is a well characterized surface membrane marker of EBV-B cells. After 30 min incubation on ice, cells are washed twice with 1 ml cold PBS+5% BSA. Streptavidin (1 μl of 0.07 μg/ml) is added for another 30 min incubation followed by two more washes. Finally, a biotinylated monomer of H-2D^{d} bound to an immunodominant HIV peptide (p18) is added for a 30 min incubation. The complex of biotinylated-anti-CD19: streptavidin: H-2D^{d}/p18 is assembled step-wise in a 4:1:4 molar ratio. Samples are washed and resuspended in a final volume of 100 μl RPMI-1640 complete medium and transferred to a 96 well plate. Either T cells specific for the immunodominant gp160 epitope, p18, in association with H-2D^{d} or control T cells specific for an unrelated peptide in association with H-2K^{d} (BCA39) are added at 10⁵ cells/well in 100 μl complete medium. Induction of IFNγ secretion by T cells is determined by IFNγ-specific ELISA assay following an overnight incubation. The data show the mean and standard deviation of relative IFNγ secretion as OD 450-OD 570 employing a standard ELISA assay protocol. Each measurement is a replicate of 4 wells. Background secretion in the absence of the assembled MHC:peptide complex is subtracted. The difference in the induction of IFNγ secretion by specific and control T cells is significant with p<0.0l by Student's single tail T test. gp160-specific T cells had a relative IFNγ secretion of 0.94 (M26). BCA39-specific T cells had a relative IFNγ secretion of 0.29 (M19).

It will be clear that the invention may be practiced otherwise than as particularly described in the foregoing description and examples.

Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

1. A compound comprising:
(a) one or more peptide-MHC Class I complexes; and
(b) an antibody or a fragment thereof specific for a cell surface marker;
wherein said peptide-MHC Class I complexes comprise an MHC Class I α chain or fragment thereof, a modified β₂-microglobulin molecule or fragment thereof, and an antigenic peptide bound in the MHC groove; and
wherein said β₂-microglobulin molecule or fragment thereof is linked to the antibody or fragment thereof; and
wherein said β₂-microglobulin or fragment has been modified in such a way as to have higher affinity for MHC Class I α than native β₂-microglobulin.

2. The compound of claim 1, wherein said β₂-microglobulin molecule or fragment thereof is linked to the amino terminus of said antibody or fragment thereof.

3. The compound of claim 2, wherein said β₂-microglobulin molecule or fragment thereof is linked to the light chain of said antibody or fragment thereof.

4. The compound of claim 2, wherein said β₂-microglobulin molecule or fragment thereof is linked to the heavy chain of said antibody or fragment thereof.

5. The compound of claim 1, wherein said β₂-microglobulin molecule or fragment thereof is linked to the carboxyl terminus of said antibody or fragment thereof.

6. The compound of claim 5, wherein said β₂-microglobulin molecule or fragment thereof is linked to the light chain of said antibody or fragment thereof.

7. The compound of claim 5, wherein said β₂-microglobulin molecule or fragment thereof is linked to the heavy chain of said antibody or fragment thereof.

8. The compound of claim 1, wherein said cell surface marker is a cell surface marker of a professional antigen presenting cell.

9. The compound of claim 8, wherein said professional antigen presenting cell is a dendritic cell.

10. The compound of claim 9, wherein said cell surface marker is selected from the group consisting of CD83, CMRF-44, CMRF-56 and DEC-205.

11. The compound of claim 1, wherein said cell surface marker is a cell surface marker of a tumor cell.

12. The compound of claim 1, wherein said cell surface marker is a cell surface marker of an epithelial cell.

13. The compound of claim 1, wherein said cell surface marker is a cell surface marker of a fibroblast.

14. The compound of claim 1, wherein said cell surface marker is a cell surface marker of a T cell.

15. The compound of claim 14, wherein said cell surface marker is selected from the group consisting of CD28, CTLA-4 and CD25.

16. The compound of claim 1, wherein said antigenic peptide is derived from a cancer cell.

17. The compound of claim 1, wherein said antigenic peptide is derived from an infectious agent or from infected cells.

18. The compound of claim 1, wherein said antigenic peptide is derived from the target tissue of an autoimmune disease.

19. The compound of claim 11, wherein said antigenic peptide is derived from a cancer cell.

20. The compound of claim 1, wherein said β₂-microglobulin molecule or fragment thereof is directly fused to said antibody or fragment thereof.

21. The compound of claim 1, wherein said β₂-microglobulin molecule or fragment thereof is fused to the antigenic peptide.

22. A method of immunizing an animal, comprising administering to said animal the compound of claim 1.

23. A compound comprising:
(a) one or more peptide-MHC Class I complexes; and
(b) an antibody or fragment thereof specific for a cell surface marker;
wherein said peptide-MHC Class I complexes comprise an MHC Class Iα chain or fragment thereof, a <2-microglobulin molecule or fragment thereof, and an antigenic peptide linked to the <2-microglobulin molecule or fragment thereof and bound in the MHC groove; and
wherein said <2-microglobulin molecule or fragment thereof is linked to the antibody or fragment thereof.

24. The compound of claim 23, wherein said β₂-microglobulin molecule or fragment thereof is linked to the amino terminus of said antibody or fragment thereof.

25. The compound of claim 24, wherein said β₂-microglobulin molecule or fragment thereof is linked to the light chain of said antibody or fragment thereof.

26. The compound of claim 24, wherein said β₂-microglobulin molecule or fragment thereof is linked to the heavy chain of said antibody or fragment thereof.

27. The compound of claim 23, wherein said β₂-microglobulin molecule or fragment thereof is linked to the carboxyl terminus of said antibody or fragment thereof.

28. The compound of claim 27, wherein said β₂-microglobulin molecule or fragment thereof is linked to the light chain of said antibody or fragment thereof.

29. The compound of claim 27, wherein said β₂-microglobulin is human and has a serine to valine mutation at position 55.

30. The compound of claim 1, wherein said cell surface marker is a cell surface marker of a professional antigen presenting cell.

31. The compound of claim 30, wherein said professional antigen presenting cell is a dendritic cell.

32. The compound of claim 31, wherein said cell surface marker is selected from the group consisting of CD83, CMRF-44, CMRF-56 and DEC-205.

33. The compound of claim 23, wherein said cell surface marker is a cell surface marker of a tumor cell.

34. The compound of claim 23, wherein said cell surface marker is a cell surface marker of an epithelial cell.

35. The compound of claim 23, wherein said cell surface marker is a cell surface marker of a fibroblast.

36. The compound of claim 23, wherein said cell surface marker is a cell surface marker of a T cell.

37. The compound of claim 36, wherein said cell surface marker is selected from the group consisting of CD28, CTLA-4 and CD25.

38. The compound of claim 37, wherein said antigenic peptide is derived from a cancer cell.

39. The compound of claim 23, wherein said antigenic peptide is derived from an infectious agent or from infected cells.

40. The compound of claim 23, wherein said antigenic peptide is derived from the target tissue of an autoimmune disease.

41. The compound of claim 33, wherein said antigenic peptide is derived from a cancer cell.

42. The compound of claim 23, wherein said β₂-microglobulin molecule or fragment thereof is directly fused to said antibody or fragment thereof.

43. A method of immunizing an animal, comprising administering to said animal the compound of claim 23.
